SEQUENCE LISTING (46 sequences)

SEQ ID NO: 1 - protein, 10 aa, synthetic construct
GYTFTGYYIH

SEQ ID NO: 2 - protein, 17 aa, synthetic construct
WINPYTGGTNYAQKFQG

SEQ ID NO: 3 - protein, 12 aa, synthetic construct
DLYGSSNYGGDV

SEQ ID NO: 4 - protein, 11 aa, synthetic construct
QASQGISNYLN

SEQ ID NO: 5 - protein, 7 aa, synthetic construct
DASNLET

SEQ ID NO: 6 - protein, 9 aa, synthetic construct
QQYDKLPLT

SEQ ID NO: 7 - protein, 121 aa, synthetic construct
QVQLVQSGAEVKKPGASVKVSCKASGYTFTGYYIHWVRQAPGQGLEWMGWINPYTGGTNYAQKFQGRVTMTRDTSISTAYMELSRLRSDDTAVYYCARDLYGSSNYGGDVWGQGTTVTVSS

SEQ ID NO: 8 - protein, 107 aa, synthetic construct
DIQMTQSPSSLSASVGDRVTITCQASQGISNYLNWYQQKPGKAPKLLIYDASNLETGVPSRFSGSGSGTDFTFTISSLQPEDIATYYCQQYDKLPLTFGGGTKVEIK

SEQ ID NO: 9 - protein, 451 aa, synthetic construct
QVQLVQSGAEVKKPGASVKVSCKASGYTFTGYYIHWVRQAPGQGLEWMGWINPYTGGTNYAQKFQGRVTMTRDTSISTAYMELSRLRSDDTAVYYCARDLYGSSNYGGDVWGQGTTVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDICVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
DIQMTQSPSSLSASVGDRVTITCQASQGISNYLNWYQQKPGKAPKLLIYDASNLETGVPSRFSGSGSGTDFTFTISSLQPEDIATYYCQQYDKLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1353 bp, synthetic construct
  source 1..1353: mol_type = other DNA
caggtgcagctggtgcagtctggggctgaggtgaagaagcctggggcctcagtgaaggtctcctgcaaggcttctggatacaccttcaccggctactatatacactgggtgcgacaggcccctggacaagggcttgagtggatgggatggatcaacccttacaccggtggcacaaactatgcacagaagtttcagggcagggtcaccatgaccagggacacgtccatcagcacagcctacatggagctgagcaggctgagatctgacgacacggccgtgtattactgtgcgagagatctctatggttcgagtaattacggtggcgacgtctggggccaagggaccacggtcaccgtctcctcagctagcaccaagggcccatgtgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatctgcgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 12 - DNA, 642 bp, synthetic construct
  source 1..642: mol_type = other DNA
gacatccagatgacccagtctccatcctccctgtctgcatctgtaggagacagagtcaccatcacttgccaggcgagtcagggcattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataagctcccgctcactttcggcggagggaccaaggtggagatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 13 - protein, 12 aa, synthetic construct
DLYGSSNYGMDV

SEQ ID NO: 14 - protein, 11 aa, synthetic construct
QASQGIRNYLN

SEQ ID NO: 15 - protein, 9 aa, synthetic construct
QQYDNLPLT

SEQ ID NO: 16 - protein, 121 aa, synthetic construct
QVQLVQSGAEVKKPGASVKVSCKASGYTFTGYYIHWVRQAPGQGLEWMGWINPYTGGTNYAQKFQGRVTMTRDTSISTAYMELSRLRSDDTAVYYCARDLYGSSNYGMDVWGQGTTVTVSS

SEQ ID NO: 17 - protein, 107 aa, synthetic construct
DIQMTQSPSSLSASVGDRVTITCQASQGIRNYLNWYQQKPGKAPKLLIYDASNLETGVPSRFSGSGSGTDFTFTISSLQPEDIATYYCQQYDNLPLTFGGGTKVEIK

SEQ ID NO: 18 - protein, 451 aa, synthetic construct
QVQLVQSGAEVKKPGASVKVSCKASGYTFTGYYIHWVRQAPGQGLEWMGWINPYTGGTNYAQKFQGRVTMTRDTSISTAYMELSRLRSDDTAVYYCARDLYGSSNYGMDVWGQGTTVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDICVEWESNGQPENNYKTTPPVLDSDGSFFLYSKLTVDKSRWQQGNVFSCSVMHEALHNHYTQKSLSLSPGK

SEQ ID NO: 19 - protein, 214 aa, synthetic construct
DIQMTQSPSSLSASVGDRVTITCQASQGIRNYLNWYQQKPGKAPKLLIYDASNLETGVPSRFSGSGSGTDFTFTISSLQPEDIATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - DNA, 1353 bp, synthetic construct
  source 1..1353: mol_type = other DNA
caggtgcagctggtgcagtctggggctgaggtgaagaagcctggggcctcagtgaaggtctcctgcaaggcttctggatacaccttcaccggctactatatacactgggtgcgacaggcccctggacaagggcttgagtggatgggatggatcaacccttacaccggtggcacaaactatgcacagaagtttcagggcagggtcaccatgaccagggacacgtccatcagcacagcctacatggagctgagcaggctgagatctgacgacacggccgtgtattactgtgcgagagatctctatggttcgagtaattacggtatggacgtctggggccaagggaccacggtcaccgtctcctcagctagcaccaagggcccatgcgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatctgcgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 21 - DNA, 642 bp, synthetic construct
  source 1..642: mol_type = other DNA
gacatccagatgacccagtctccatcctccctgtctgcatctgtaggagacagagtcaccatcacttgccaggcgagtcagggcattcgcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataacctcccgctcactttcggcggagggaccaaggtggagatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 22 - protein, 10 aa, synthetic construct
GYTFTGYYMH

SEQ ID NO: 23 - protein, 17 aa, synthetic construct
WINPYTGGTKYAQKFQG

SEQ ID NO: 24 - protein, 121 aa, synthetic construct
QVQLVQSGAEVKKPGASVKVSCKASGYTFTGYYMHWVRQAPGQGLEWMGWINPYTGGTKYAQKFQGRVTMTRDTSISTAYMELSRLRSDDTAVYYCARDLYGSSNYGMDVWGQGTTVTVSS

SEQ ID NO: 25 - protein, 451 aa, synthetic construct
QVQLVQSGAEVKKPGASVKVSCKASGYTFTGYYMHWVRQAPGQGLEWMGWINPYTGGTKYAQKFQGRVTMTRDTSISTAYMELSRLRSDDTAVYYCARDLYGSSNYGMDVWGQGTTVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDICVEWESNGQPENNYKTTPPVLDSDGSFFLYSKLTVDKSRWQQGNVFSCSVMHEALHNHYTQKSLSLSPGK

SEQ ID NO: 26 - DNA, 1353 bp, synthetic construct
  source 1..1353: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaacccttacaccggtggcacaaagtatgcacagaagtttcagggcagggtcaccatgaccagggacacgtccatcagcacagcctacatggagctgagcaggctgagatctgacgacacggccgtgtattactgtgcgagagatctctatggttcgagtaattacggtatggacgtctggggccaagggaccacggtcaccgtctcctcagctagcaccaagggcccatgcgtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatctgcgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 27 - protein, 107 aa, synthetic construct
DIQMTQSPSSLSASVGDRVTITCQASQGIRNYLNWYQQKPGKAPKLLIYDASNLETGVPSRFSGSGSGTDFTFTISSLQPEDIATYYCQQYDKLPLTFGGGTKVEIK

SEQ ID NO: 28 - protein, 214 aa, synthetic construct
DIQMTQSPSSLSASVGDRVTITCQASQGIRNYLNWYQQKPGKAPKLLIYDASNLETGVPSRFSGSGSGTDFTFTISSLQPEDIATYYCQQYDKLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 642 bp, synthetic construct
  source 1..642: mol_type = other DNA
gacatccagatgacccagtctccatcctccctgtctgcatctgtaggagacagagtcaccatcacttgccaggcgagtcagggcattcgcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataagctcccgctcactttcggcggagggaccaaggtggagatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 30 - protein, 12 aa, synthetic construct
DIYGSSNYGGDV

SEQ ID NO: 31 - protein, 11 aa, synthetic construct
QASQDISNYLN

SEQ ID NO: 32 - protein, 9 aa, synthetic construct
QQYDTLPLT

SEQ ID NO: 33 - protein, 121 aa, synthetic construct
QVQLVQSGAEVKKPGASVKVSCKASGYTFTGYYIHWVRQAPGQGLEWMGWINPYTGGTNYAQKFQGRVTMTRDTSISTAYMELSRLRSDDTAVYYCARDIYGSSNYGGDVWGQGTTVTVSS

SEQ ID NO: 34 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDTLPLTFGGGTKVEIK

SEQ ID NO: 35 - protein, 451 aa, synthetic construct
QVQLVQSGAEVKKPGASVKVSCKASGYTFTGYYIHWVRQAPGQGLEWMGWINPYTGGTNYAQKFQGRVTMTRDTSISTAYMELSRLRSDDTAVYYCARDIYGSSNYGGDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDTLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 1353 bp, synthetic construct
  source 1..1353: mol_type = other DNA
caggtgcagctggtgcagtctggggctgaggtgaagaagcctggggcctcagtgaaggtctcctgcaaggcttctggatacaccttcaccggctactatatacactgggtgcgacaggcccctggacaagggcttgagtggatgggatggatcaacccttacaccggtggcacaaactatgcacagaagtttcagggcagggtcaccatgaccagggacacgtccatcagcacagcctacatggagctgagcaggctgagatctgacgacacggccgtgtattactgtgcgagagatatctatggttcgagtaattacggtggcgacgtctggggccaagggaccacggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaa

SEQ ID NO: 38 - DNA, 642 bp, synthetic construct
  source 1..642: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataccctcccgctcactttcggcggagggaccaaggtggagatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 39 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 40 - protein, 233 aa, Macaca mulatta
MSTESMIRDVELAEEALPRKTAGPQGSRRCWFLSLFSFLLVAGATTLFCLLHFGVIGPQREEFPKDPSLISPLAQAVRSSSRTPSDKPVAHVVANPQAEGQLQWLNRRANALLANGVELTDNQLVVPSEGLYLIYSQVLFKGQGCPSNHVLLTHTISRIAVSYQTKVNLLSAIKSPCQRETPEGAEAKPWYEPIYLGGVFQLEKGDRLSAEINLPDYLDFAESGQVYFGIIAL

SEQ ID NO: 41 - protein, 233 aa, Canis familiaris
MSTESMIRDVELAEEPLPKKAGGPPGSRRCFCLSLFSFLLVAGATTLFCLLHFGVIGPQREELPNGLQLISPLAQTVKSSSRTPSDKPVAHVVANPEAEGQLQWLSRRANALLANGVELTDNQLIVPSDGLYLIYSQVLFKGQGCPSTHVLLTHTISRFAVSYQTKVNLLSAIKSPCQRETPEGTEAKPWYEPIYLGGVFQLEKGDRLSAEINLPNYLDFAESGQVYFGIIAL

SEQ ID NO: 42 - protein, 233 aa, Macaca fascicularis
MSTESMIQDVELAEEALPRKTAGPQGSRRCWFLSLFSFLLVAGAATLFCLLHFGVIGPQREEFPKDPSLISPLAQAVRSSSRTPSDKPVAHVVANPQAEGQLQWLNRRANALVANGVELTDNQLVVPSEGLYLIYSQVLFKGQGCPSNHVLLTHTISRIAVSYQTKVNLLSAIKSPCQRETPEGAEAKPWYEPIYLGGVFQLEKGDRLSAEINLPDYLDFAESGQVYFGIIAL

SEQ ID NO: 43 - protein, 11 aa, synthetic construct
  VARIANT 7: note = X at position 7 is Serine or Arginine
QASQGIXNYLN

SEQ ID NO: 44 - protein, 9 aa, synthetic construct
  VARIANT 5: note = X at position 5 is Asparagine or Lysine
QQYDXLPLT

SEQ ID NO: 45 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 46 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

(19) United States
(12) Patent Application Publication
(10) Pub. No.: US 2024/0082414 A1
(43) Pub. Date: Mar. 14, 2024

(54) HUMAN TUMOR NECROSIS FACTOR ALPHA ANTIBODY GLUCOCORTICOID CONJUGATES

(71) Applicant: Eli Lilly and Company, Indianapolis, IN (US)

(72) Inventors: Grace Chao, San Diego, CA (US); Joshua R Clayton, Fishers, IN (US); Jordan Scott Crampton, San Diego, CA (US); Yiqing Feng, Carmel, IN (US); Donmienne Doen Mun Leung, Saltburn-by-the-sea (GB); Songqing Na, San Diego, CA (US); Kristin Paige Newburn, Indianapolis, IN (US); Scott Charles Potter, San Diego, CA (US); Bharathi Ramamurthy, San Diego, CA (US); David John Stokell, Indianapolis, IN (US); Jacqueline M. Wurst, Zionsville, IN (US); Jianghuai Xu, San Diego, CA (US)

(21) Appl. No.: 18/312,653

(22) Filed: May 5, 2023

Related U.S. Application Data
(60) Provisional application No. 63/341,691, filed on May 13, 2022.

(51) Int. Cl. A61K 47/68; A61K 31/58; A61P 19/02; C07K 16/24

(52) U.S. Cl. CPC A61K 47/6803; A61K 31/58; A61P 19/02; C07K 16/241

(57) ABSTRACT

The present disclosure provides human tumor necrosis factor alpha antibody glucocorticoid receptor agonist conjugates and methods of using the conjugates for the treatment of autoimmune and inflammatory diseases.

### SEQUENCE LISTING FILE

The present application is being filed along with a Sequence Listing in ST.26 XML format. The Sequence Listing is provided as a file titled “22154_US sequence listing” created Mar. 24, 2023, and is 54 kilobytes in size. The Sequence Listing information in the ST.26 XML format is incorporated herein by reference in its entirety.

### FIELD OF THE INVENTION

The present disclosure provides human tumor necrosis factor alpha antibody glucocorticoid receptor agonist conjugates, methods of using the conjugates for the treatment of autoimmune and inflammatory diseases, such as rheumatoid arthritis, psoriatic arthritis, Crohn's disease, ulcerative colitis, plaque psoriasis, and ankylosing spondylitis, processes for preparing the conjugates, and pharmaceutical compositions comprising the human TNFα antibody glucocorticoid conjugates.

### BACKGROUND OF THE INVENTION

Rheumatoid arthritis (RA) is a debilitating chronic autoimmune disease which attacks the joints, most commonly joints in the hands, wrists, and knees, and usually attacks many joints at once. In RA, the lining of the joint becomes inflamed, causing damage to joint tissue which can cause stiffness, swelling, unsteadiness (lack of balance), deformity, and chronic pain. Current treatments employ, for example, non-steroidal anti-inflammatory drugs (NSAIDSs), corticosteroids, disease modifying anti-rheumatic drugs (DMARDs), antibody therapeutics, such as methotrexate, tofacitinib, Etanercept, adalimumab, infliximab, golimumab, and certolizumab. Shortcomings of such treatments include for example, non-target toxicities and development of anti-drug antibodies.

Anti-drug antibodies may be non-neutralizing antibodies that bind to the anti-TNFα antibody therapeutic simultaneously with TNFα, or they may be neutralizing antibodies which reduce the effective concentrations of the anti-TNFα therapeutic antibody in the serum and/or compete with TNFα for the antigen-binding site (paratope) thus inhibiting the working mechanism of the anti-TNFα therapeutic antibody. (van Schie K A, et al, Annals of the Rheumatic Diseases, 2015, 74:311-314). For example, studies have shown that greater than ninety percent of anti-TNFα drug antibodies are neutralizing and may be cross reactive to other anti-TNFα antibody therapeutics. (van Schie K A, et al, 2015). As such, in some instances, patients developing anti-drug antibodies to anti-TNFα antibodies have been reported to have, diminished clinical response to these therapeutics and/or adverse events such as infusion related reactions characterized by symptoms such as fever, pruritus, bronchospasms, or cardiovascular collapse during or within the first day after drug administration (Atiqi, S., Front Immunol., 2020, 26(11):312). Thus, there remains a significant need for new agents that provide improved effective treatment of inflammatory and/or autoimmune diseases, such as RA, and which minimize or eliminate the disadvantages possessed by currently approved treatments.

WO2017/210471 discloses certain glucocorticoid receptor agonists (GC), antibodies, and immunoconjugates thereof. WO2018/089373 discloses novel steroids, protein conjugates thereof, and methods for treating diseases, disorders, and conditions comprising administering the steroids and conjugates. To date, there are no approved human TNFα antibody GC conjugates for the treatment of diseases.

### SUMMARY OF THE INVENTION

The present disclosure provides certain novel human TNFα antibody GC conjugates wherein the antibody binds to human TNFα. The present disclosure further provides compositions comprising novel anti-human TNFα antibody GC conjugates and methods of using such anti-human TNFα antibody GC conjugates and compositions thereof. In addition, the present disclosure provides certain novel anti-human TNFα antibody GC conjugates useful in the treatment of autoimmune diseases and inflammatory diseases such as rheumatoid arthritis. The present disclosure further provides certain novel anti-human TNFα antibody GC conjugates useful in the treatment of autoimmune diseases and inflammatory diseases in patients who have developed anti-drug antibodies against other anti-TNFα therapeutics (e.g., adalimumab). Certain anti-human TNFα antibody GC conjugates disclosed herein, present good developability profiles such as good physical-chemical properties (e.g., low viscosity, or aggregation, good thermal stability) to facilitate development, manufacturing, and formulation. As such, certain anti-human TNFα antibody GC conjugates provided herein have one or more of the following properties: 1) bind human TNFα with desirable potency, 2) bind human membrane TNFα and internalize into the cell, 3) bind rhesus macaque monkey, and/or canine TNFα with desirable potency, 4) inhibit soluble and membrane human TNFα induced apoptosis, 5) modulate both TNFR and glucocorticoid receptor mediated cytokine expression (e.g., inhibit IL-13, IL-6, GM-CSF, induce IL-10) in vitro, 6) inhibit TNFR mediated cytokine expression (e.g., CXCL1) in vivo, 7) induce ADCC activity, 8) exhibit low to no cross-reactivity to anti-drug antibodies against other anti-TNFα therapeutic (e.g., adalimumab) in vivo and in vitro, 9) significantly inhibit tissue and/or polyarthritis joint inflammation in vivo, 10) significantly inhibit joint inflammation in adalimumab refractory mice in vivo, or 11) have a good developability profile e.g., acceptable viscosity, solubility and aggregation, good stability, and/or acceptable pharmacokinetic profile to facilitate development, manufacturing, and/or formulation.

Accordingly, in one embodiment, the disclosure provides a conjugate of Formula I:

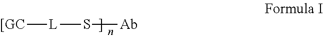

wherein Ab is an antibody that binds human tumor necrosis factor alpha (“anti-human TNFα antibody”), wherein Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1 or 22;
the HCDR2 comprises SEQ ID NO: 2 or 23;
the HCDR3 comprises SEQ ID NO: 3, 13, or 30;
the LCDR1 comprises SEQ ID NO: 4, 14, 31, or 43;
the LCDR2 comprises SEQ ID NO: 5; and
the LCDR3 comprises SEQ ID NO: 6, 15, 32, or 44;

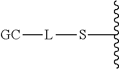

is:

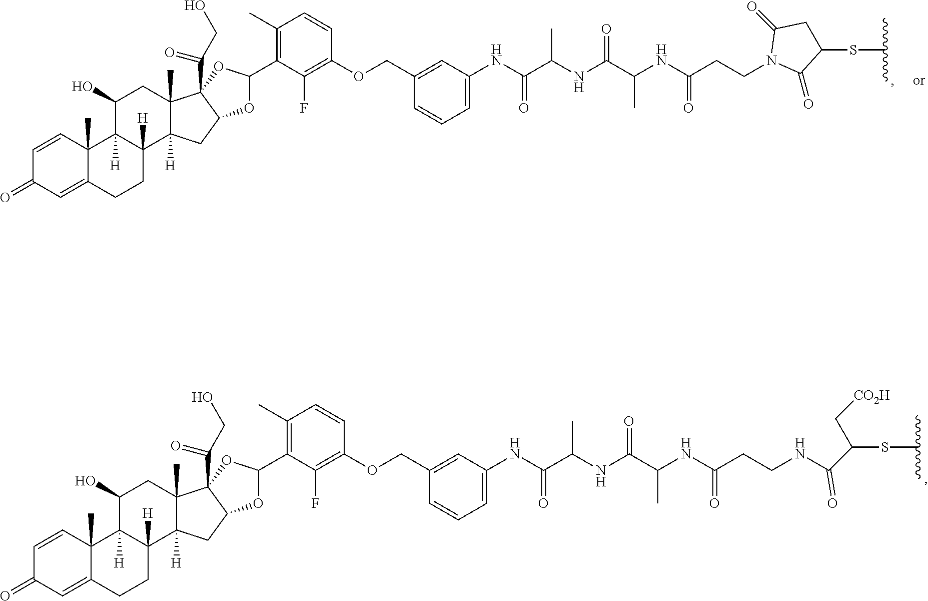

and n is 1-5.

In a further embodiment, the disclosure provides a conjugate of Formula Ia:

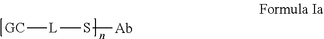

wherein Ab is an antibody that binds human TNFα, wherein Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1 or 22;
the HCDR2 comprises SEQ ID NO: 2 or 23;
the HCDR3 comprises SEQ ID NO: 3, 13, or 30;
the LCDR1 comprises SEQ ID NO: 4, 14, 31, or 43;
the LCDR2 comprises SEQ ID NO: 5; and
the LCDR3 comprises SEQ ID NO: 6, 15, 32, or 44;

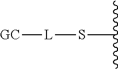

is:

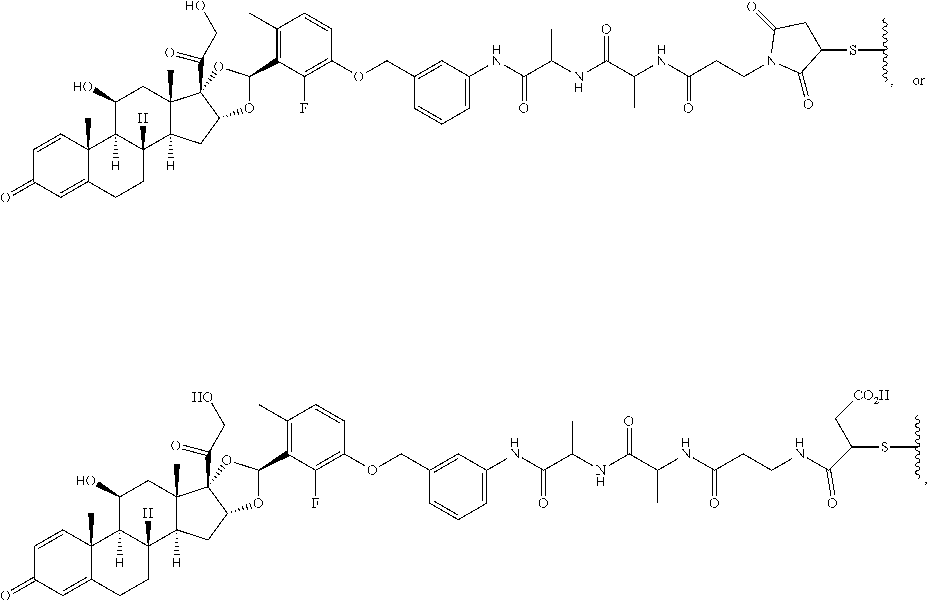

and n is 1-5.

In a further embodiment, the disclosure provides a conjugate of Formula Ib:

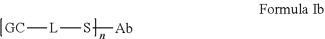

wherein Ab is an antibody that binds human TNFα, wherein Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1 or 22;
the HCDR2 comprises SEQ ID NO: 2 or 23;
the HCDR3 comprises SEQ ID NO: 3, 13, or 30;
the LCDR1 comprises SEQ ID NO: 4, 14, 31, or 43;
the LCDR2 comprises SEQ ID NO: 5; and
the LCDR3 comprises SEQ ID NO: 6, 15, 32, or 44;

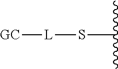

is:

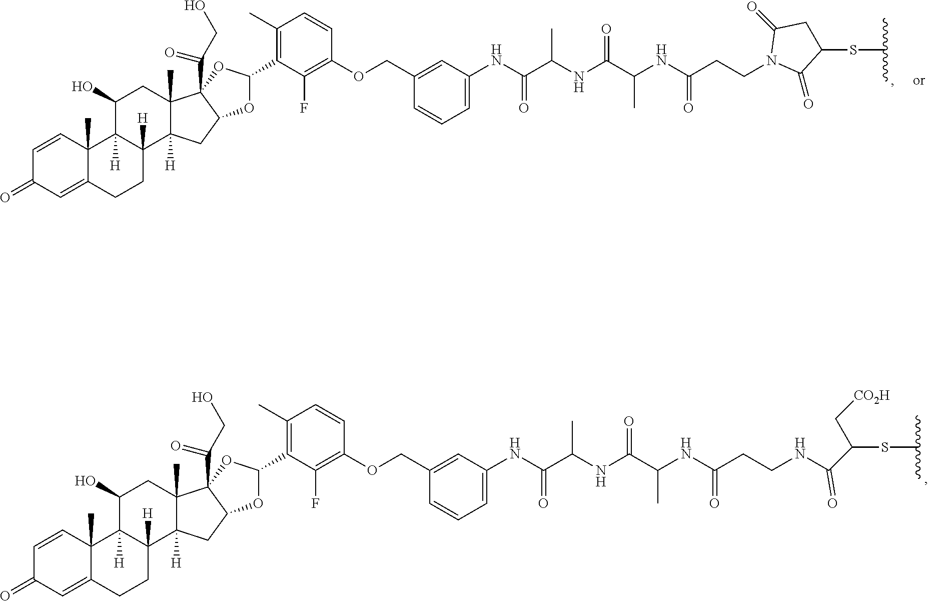

and n is 1-5.

In a further embodiment, the disclosure provides a conjugate of Formula Ic:

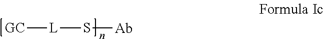

wherein Ab is an antibody that binds human TNFα, wherein Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1 or 22;
the HCDR2 comprises SEQ ID NO: 2 or 23;
the HCDR3 comprises SEQ ID NO: 3, 13, or 30;
the LCDR1 comprises SEQ ID NO: 4, 14, 31, or 43;
the LCDR2 comprises SEQ ID NO: 5; and
the LCDR3 comprises SEQ ID NO: 6, 15, 32, or 44;

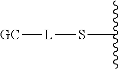

is:

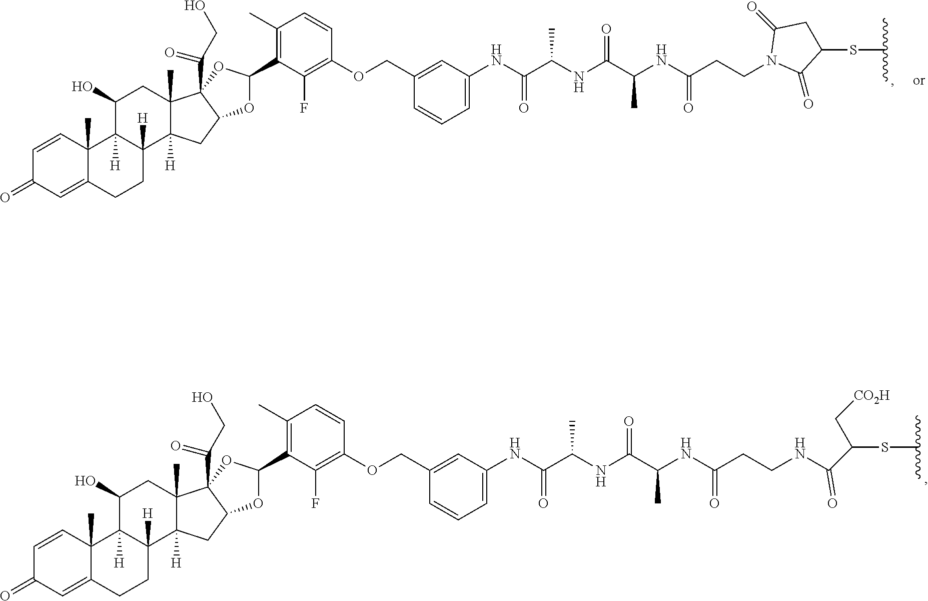

and n is 1-5.

In a further embodiment, the disclosure provides a conjugate of Formula Id:

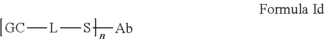

wherein Ab is an antibody that binds human TNFα, wherein Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1 or 22;
the HCDR2 comprises SEQ ID NO: 2 or 23;
the HCDR3 comprises SEQ ID NO: 3, 13, or 30;
the LCDR1 comprises SEQ ID NO: 4, 14, 31, or 43;
the LCDR2 comprises SEQ ID NO: 5; and
the LCDR3 comprises SEQ ID NO: 6, 15, 32, or 44;

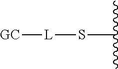

is:

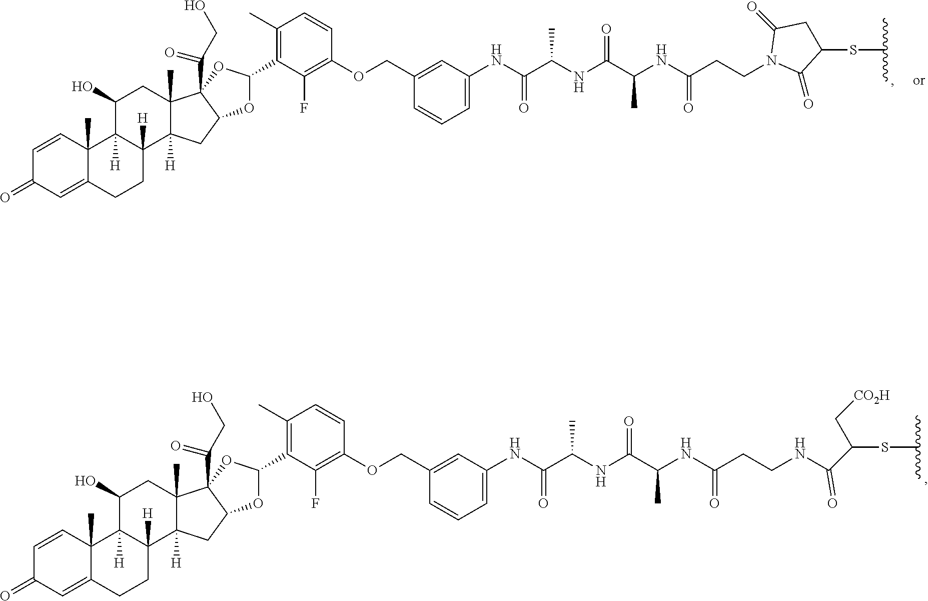

and n is 1-5.

In a further embodiment, the disclosure provides a conjugate of Formula Ie:

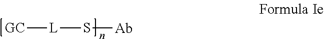

wherein Ab is an antibody that binds human TNFα, wherein Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1, or 22;
the HCDR2 comprises SEQ ID NO: 2, or 23;
the HCDR3 comprises SEQ ID NO: 3, 13, or 30;
the LCDR1 comprises SEQ ID NO: 4, 14, 31, or 43;
the LCDR2 comprises SEQ ID NO: 5; and
the LCDR3 comprises SEQ ID NO: 6, 15, 32 or 44;

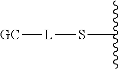

is:

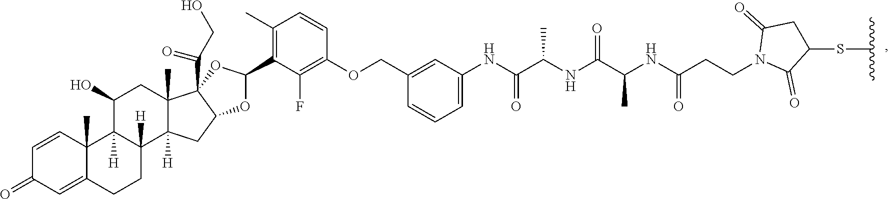

and n is 1-5.

In a further embodiment, the disclosure provides a conjugate of Formula If:

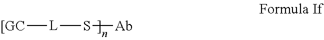

wherein Ab is an antibody that binds human TNFα, wherein Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1 or 22;
the HCDR2 comprises SEQ ID NO: 2 or 23;
the HCDR3 comprises SEQ ID NO: 3, 13, or 30;
the LCDR1 comprises SEQ ID NO: 4, 14, 31, or 43;
the LCDR2 comprises SEQ ID NO: 5; and
the LCDR3 comprises SEQ ID NO: 6, 15, 32, or 44;

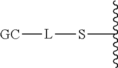

is:

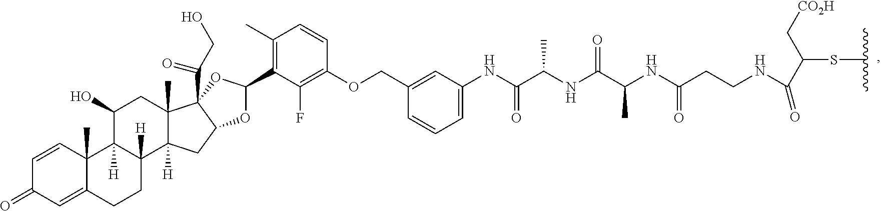

and n is 1-5.

In some embodiments, the conjugate of Formula I wherein the antibody (“Ab”) that binds human TNFα is Ab1, wherein Ab1 comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 1, the HCDR2 comprises SEQ ID NO: 2, the HCDR3 comprises SEQ ID NO: 3, the LCDR1 comprises SEQ ID NO: 4, the LCDR2 comprises SEQ ID NO: 5, and the LCDR3 comprises SEQ ID NO: 6. In some embodiments, the Ab1 comprises a VH comprising SEQ ID NO: 7 and a VL comprising SEQ ID NO: 8. In some embodiments, the Ab1 comprises a heavy chain (HC) comprising SEQ ID NO: 9 and a light chain (LC) comprising SEQ ID NO: 10.

In some embodiments, the conjugate of Formula I wherein the antibody (“Ab”) that binds human TNFα is Ab2, wherein Ab2 comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 1, the HCDR2 comprises SEQ ID NO: 2, the HCDR3 comprises SEQ ID NO: 13, the LCDR1 comprises SEQ ID NO: 14, the LCDR2 comprises SEQ ID NO: 5, and the LCDR3 comprises SEQ ID NO: 15. In some embodiments, the Ab2 comprises a VH comprising SEQ ID NO: 16 and a VL comprising SEQ ID NO: 17. In some embodiments, the Ab2 comprises a heavy chain (HC) comprising SEQ ID NO: 18 and a light chain (LC) comprising SEQ ID NO: 19.

In some embodiments, the conjugate of Formula I wherein the antibody (“Ab”) that binds human TNFα is Ab3, wherein Ab3 comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 22, the HCDR2 comprises SEQ ID NO: 23, the HCDR3 comprises SEQ ID NO: 13, the LCDR1 comprises SEQ ID NO: 4, the LCDR2 comprises SEQ ID NO: 5, and the LCDR3 comprises SEQ ID NO: 6. In some embodiments, the Ab3 comprises a VH comprising SEQ ID NO: 24 and a VL comprising SEQ ID NO: 8. In some embodiments, the Ab3 comprises a heavy chain (HC) comprising SEQ ID NO: 25 and a light chain (LC) comprising SEQ ID NO: 10.

In some embodiments, the conjugate of Formula I wherein the antibody (“Ab”) that binds human TNFα is Ab4, wherein Ab4 comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 22, the HCDR2 comprises SEQ ID NO: 23, the HCDR3 comprises SEQ ID NO: 13, the LCDR1 comprises SEQ ID NO: 14, the LCDR2 comprises SEQ ID NO: 5, and the LCDR3 comprises SEQ ID NO: 15. In some embodiments, Ab4 comprises a VH comprising SEQ ID NO: 24 and a VL comprising SEQ ID NO: 17. In some embodiments, the Ab4 comprises a heavy chain (HC) comprising SEQ ID NO: 25 and a light chain (LC) comprising SEQ ID NO: 19.

In some embodiments, the conjugate of Formula I wherein the antibody (“Ab”) that binds human TNFα is Ab5, wherein Ab5 comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 22, the HCDR2 comprises SEQ ID NO: 23, the HCDR3 comprises SEQ ID NO: 13, the LCDR1 comprises SEQ ID NO: 14, the LCDR2 comprises SEQ ID NO: 5, and the LCDR3 comprises SEQ ID NO: 6. In some embodiments, the Ab5 comprises a VH comprising SEQ ID NO: 24 and a VL comprising SEQ ID NO: 27. In some embodiments, the Ab5 comprises a heavy chain (HC) comprising SEQ ID NO: 25 and a light chain (LC) comprising SEQ ID NO: 28.

In some embodiments, the conjugate of Formula I wherein the antibody (“Ab”) that binds human TNFα is Ab6, wherein Ab6 comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 1, the HCDR2 comprises SEQ ID NO: 2, the HCDR3 comprises SEQ ID NO: 30, the LCDR1 comprises SEQ ID NO: 31, the LCDR2 comprises SEQ ID NO: 5, and the LCDR3 comprises SEQ ID NO: 32. In some embodiments, the Ab6 comprises a VH comprising SEQ ID NO: 33 and a VL comprising SEQ ID NO: 34. In some embodiments, the Ab6 comprises a heavy chain (HC) comprising SEQ ID NO: 35 and a light chain (LC) comprising SEQ ID NO: 36.

In some embodiments, the conjugate of Formula I wherein the antibody (“Ab”) that binds human TNFα comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 22, the HCDR2 comprises SEQ ID NO: 23, the HCDR3 comprises SEQ ID NO: 13, the LCDR1 comprises SEQ ID NO: 14, the LCDR2 comprises SEQ ID NO: 5, and the LCDR3 comprises SEQ ID NO: 44. In some embodiments, SEQ ID NO: 44 comprises amino acid residues QQYDXaa₅ LPLT, wherein Xaa₅ of SEQ ID NO: 44 is Asparagine or Lysine.

In some embodiments, the conjugate of Formula I wherein the antibody (“Ab”) that binds human TNFα comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1 comprises SEQ ID NO: 22, the HCDR2 comprises SEQ ID NO: 23, the HCDR3 comprises SEQ ID NO: 13, the LCDR1 comprises SEQ ID NO: 43, the LCDR2 comprises SEQ ID NO: 5, and the LCDR3 comprises SEQ ID NO: 6. In some embodiments, SEQ ID NO: 43 comprises amino acid residues QASQGIXaa₇NYLN wherein Xaa₇ of SEQ ID NO: 43 is Serine or Arginine.

In some embodiments, the conjugate of Formula I, wherein the anti-human TNFα antibody is a fully human antibody. In further embodiments, the anti-human TNFα antibody has a human IgG1 isotype.

In some embodiments of the present disclosure, the conjugate of Formula I, wherein the anti-human TNFα antibody has a modified human IgG1. In some embodiments, the modifications are in the heavy chain variable region (VH). In some embodiments, the modifications are in the light chain variable region (VL). In some embodiments, the modifications are in the VH and the VL. In further embodiments, the modified human IgG1 VH and/or VL provides a desirable viscosity profile and/or immunogenicity risk profile to the anti-human TNFα antibody of the present disclosure.

In further embodiments, the conjugate of Formula I, wherein the anti-human TNFα antibody has a modified human IgG1 constant domain comprising engineered cysteine residues for use in the generation of antibody conjugate (also referred to as bioconjugates) (see WO 2018/232088 A1). More particularly, in such embodiments of the present disclosure, the anti-human TNFα antibody comprises engineered cysteine residues in the IgG1 heavy chain. In such embodiments, the anti-human TNFα antibody comprises a cysteine at amino acid residue 124 (EU numbering) in heavy chain constant domain 1 (CH1), or a cysteine at amino acid residue 378 (EU numbering) in heavy chain constant domain 2 (CH2). In further embodiments, the anti-human TNFα antibody comprises a cysteine at amino acid residue 124 (EU numbering) in the CH1 domain and a cysteine at amino acid residue 378 (EU numbering) in the CH2 domain.

In some embodiments, the conjugate of Formula I wherein the anti-human TNFα antibody has low to no cross reactivity to anti-drug antibodies against other anti-TNFα therapeutics (e.g., adalimumab, infliximab, golimumab, certolizumab, or Etanercept) or a conjugate thereof. In particular embodiments, the conjugate of Formula I, wherein the anti-human TNFα antibody has low to no cross reactivity to anti-drug antibodies against adalimumab. In such embodiments, certain conjugates of Formula I may be used to treat patients who have developed anti-drug antibodies to prior treatment with other anti-TNFα therapeutic (e.g., adalimumab) as defined herein. In further embodiments, certain conjugate of Formula I may be used to treat patients who have developed anti-drug antibodies to other anti-TNFα therapeutics from prior treatment with such other anti-TNFα therapeutics and thus have a diminished clinical response or adverse reactions to the other anti-TNFα therapeutics. In such embodiments, the conjugate of Formula I, wherein the anti-human TNFα antibody has sufficiently different amino acid and nucleic acid sequences such that they have low to no cross reactivity to anti-drug antibodies against other anti-TNFα therapeutics. In particular embodiments, the conjugate of Formula I, wherein the anti-human TNFα antibody of the present disclosure has sufficiently different CDR amino acid sequences such that they have low to no cross reactivity to anti-drug antibodies against other anti-TNFα therapeutics. In some embodiments, the other anti-TNFα therapeutic is adalimumab, infliximab, golimumab, certolizumab, or Etanercept or a conjugate thereof.

In some embodiments, the present disclosure provides nucleic acids encoding a HC or LC, or a VH or VL, of the novel antibodies that bind anti-human TNFα, or vectors comprising such nucleic acids.

In some embodiments, the present disclosure provides a nucleic acid comprising a sequence of SEQ ID NO: 11, 12, 20, 21, 26, 29, 37, or 38.

In some embodiments, nucleic acids encoding a heavy chain or light chain of the antibodies that bind anti-human TNFα are provided. In some embodiments nucleic acids comprising a sequence encoding SEQ ID NO: 9, 10, 18, 19, 25, 28, 35, or 36 are provided. In some embodiments, nucleic acids comprising a sequence encoding an antibody heavy chain that comprises SEQ ID NO: 9, 18, 25, or 35 is provided. For example, the nucleic acid can comprise a sequence of SEQ ID NO: 11, 20, 26, or 37. In some embodiments, nucleic acids comprising a sequence encoding an antibody light chain that comprises SEQ ID NO: 10, 19, 28, or 36 is provided. For example, the nucleic acid can comprise a sequence of SEQ ID NO: 12, 21, or 29, or 38.

In some embodiments of the present disclosure, nucleic acids encoding a VH or VL of the antibodies that bind anti-human TNFα are provided. In some embodiments, nucleic acids comprising a sequence encoding SEQ ID NO: 7, 8, 16, 17, 24, 27, 33, or 34 are provided. In some embodiments, nucleic acids comprising a sequence encoding an antibody VH that comprises SEQ ID NO: 7, 16, 24, or 33 is provided. In some embodiments, nucleic acids comprising a sequence encoding an antibody VL that comprises SEQ ID NO: 8, 17, 27, or 34 is provided.

Some embodiments of the present disclosure provide vectors comprising a nucleic acid sequence encoding an antibody heavy chain or light chain. For example, such vectors can comprise a nucleic acid sequence encoding SEQ ID NO: 9, 18, 25, or 35. In some embodiments, the vector comprises a nucleic acid sequence encoding SEQ ID NO: 10, 19, 28, or 36.

Provided herein are also vectors comprising a nucleic acid sequence encoding an antibody VH or VL. For example, such vectors can comprise a nucleic acid sequence encoding SEQ ID NO: 7, 16, 24, or 33. In some embodiments, the vector comprises a nucleic acid sequence encoding SEQ ID NO: 8, 17, 27, or 34.

Provided herein are also vectors comprising a first nucleic acid sequence encoding an antibody heavy chain and a second nucleic acid sequence encoding an antibody light chain. In some embodiments, the vector comprises a first nucleic acid sequence encoding SEQ ID NO: 9, 18, 25, or 35 and a second nucleic acid sequence encoding SEQ ID NO: 10, 19, 28 or 36. In some embodiments, the vector comprises a first nucleic acid sequence encoding SEQ ID NO: 9 and a second nucleic acid sequence encoding SEQ ID NO: 10. In some embodiments, the vector comprises a first nucleic acid sequence encoding SEQ ID NO: 18 and a second nucleic acid sequence encoding SEQ ID NO: 19. In some embodiments, the vector comprises a first nucleic acid sequence encoding SEQ ID NO: 25 and a second nucleic acid sequence encoding SEQ ID NO: 10. In some embodiments, the vector comprises a first nucleic acid sequence encoding SEQ ID NO: 25 and a second nucleic acid sequence encoding SEQ ID NO: 19. In some embodiments, the vector comprises a first nucleic acid sequence encoding SEQ ID NO: 25 and a second nucleic acid sequence encoding SEQ ID NO: 28. In some embodiments, the vector comprises a first nucleic acid sequence encoding SEQ ID NO: 35 and a second nucleic acid sequence encoding SEQ ID NO: 36.

Also provided herein are compositions comprising a first vector comprising a nucleic acid sequence encoding an antibody heavy chain, and a second vector comprising a nucleic acid sequence encoding an antibody light chain. In some embodiments, the composition comprises a first vector comprising a nucleic acid sequence encoding SEQ ID NO: 9, 18, 25, or 35 and a second nucleic acid sequence encoding SEQ ID NO: 10, 19, 28 or 36. In some embodiments, the composition comprises a first vector comprising a nucleic acid sequence encoding SEQ ID NO: 9 and a second vector comprising a nucleic acid sequence encoding SEQ ID NO: 10. In some embodiments, the composition comprises a first vector comprising a nucleic acid sequence encoding SEQ ID NO: 18 and a second vector comprising a nucleic acid sequence encoding SEQ ID NO: 19. In some embodiments, the composition comprises a first vector comprising a nucleic acid sequence encoding SEQ ID NO: 25 and a second vector comprising a nucleic acid sequence encoding SEQ ID NO: 10. In some embodiments, the composition comprises a first vector comprising a nucleic acid sequence encoding SEQ ID NO: 25 and a second vector comprising a nucleic acid sequence encoding SEQ ID NO: 19. In some embodiments, the composition comprises a first vector comprising a nucleic acid sequence encoding SEQ ID NO: 25 and a second vector comprising a nucleic acid sequence encoding SEQ ID NO: 28. In some embodiments, the composition comprises a first vector comprising a nucleic acid sequence encoding SEQ ID NO: 35 and a second vector comprising a nucleic acid sequence encoding SEQ ID NO: 36.

Also provided herein are compositions comprising a vector comprising a nucleic acid sequence encoding an antibody heavy chain, and a nucleic acid sequence encoding an antibody light chain. In some embodiments, the composition comprises a vector comprising a nucleic acid sequence encoding SEQ ID NO: 9, 18, 25, or 35 and a second nucleic acid sequence encoding SEQ ID NO: 10, 19, 28 or 36.
In an embodiment, n is 2-5.
In an embodiment, n is 3-5.
In an embodiment, n is 3-4.
In an embodiment, n is about 4.
In an embodiment, n is about 3.
In an embodiment, n is about 2.

As used herein, “GC” in the Formula:

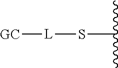

refers to a suitable glucocorticoid receptor agonist payload and includes the following Formulas IIa, IIb, and IIc:

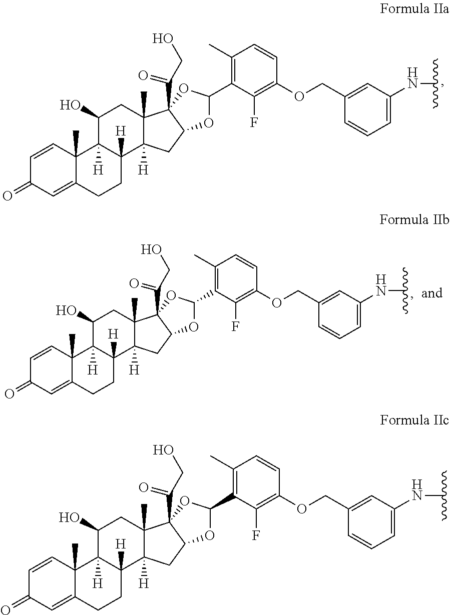

As used herein, “L” in the Formula

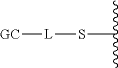

refers to a suitable linker group which connects Ab to the GC. Suitable linkers known to those of ordinary skill in the art include, for example, cleavable and noncleavable linkers. More specifically, suitable linkers “L” include the following of Formulas Ma thru

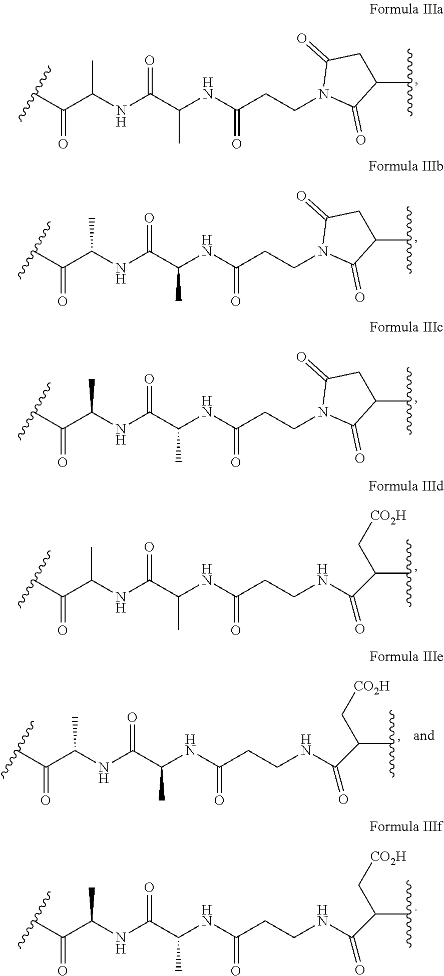 [SMILES: *C(=O)C(C)NC(=O)C(C)NC(=O)CCN1C(=O)CC(*)C1=O ; *C(=O)[C@H](C)NC(=O)[C@H](C)NC(=O)CCN1C(=O)CC(*)C1=O ; *C(=O)[C@@H](C)NC(=O)[C@@H](C)NC(=O)CCN1C(=O)CC(*)C1=O ; *C(=O)C(C)NC(=O)C(C)NC(=O)CCNC(=O)C(*)CC(=O)O ; *C(=O)[C@H](C)NC(=O)[C@H](C)NC(=O)CCNC(=O)C(*)CC(=O)O ; *C(=O)[C@@H](C)NC(=O)[C@@H](C)NC(=O)CCNC(=O)C(*)CC(=O)O]

In an embodiment, the disclosure provides a glucocorticoid receptor agonist payload-linker of Formula IV:

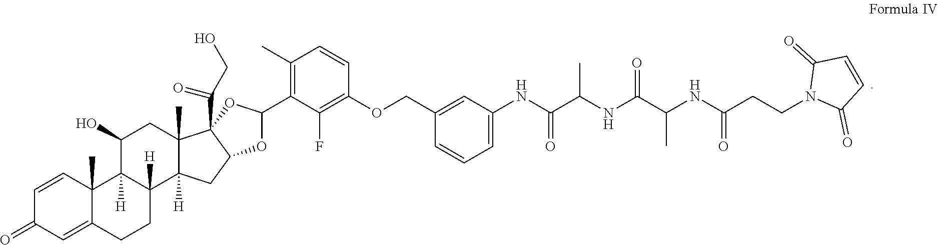

In an embodiment, the disclosure provides a glucocorticoid receptor agonist payload-linker of Formula IVa:

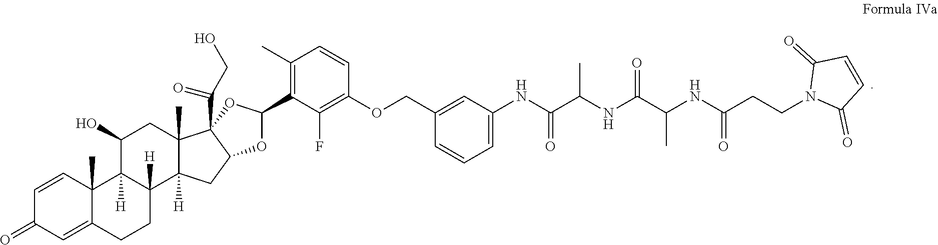

In an embodiment, the disclosure provides a glucocorticoid receptor agonist payload-linker of Formula IVb:

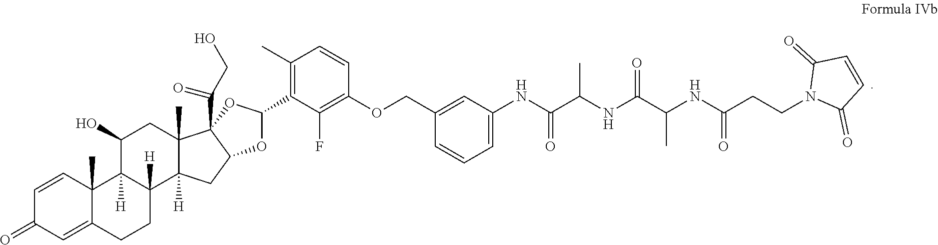

In an embodiment, the disclosure provides a glucocorticoid receptor agonist payload-linker of Formula IVc:

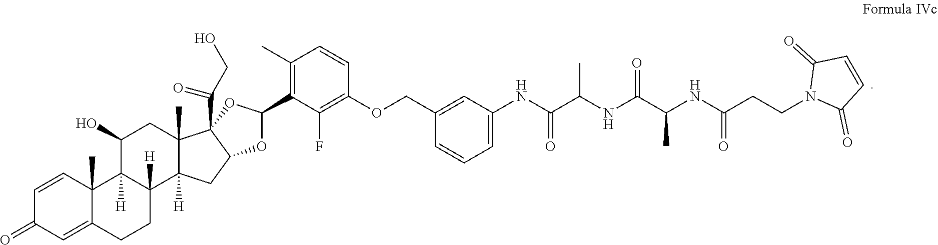

In an embodiment, the disclosure provides a glucocorticoid receptor agonist payload-linker of Formula IVd:

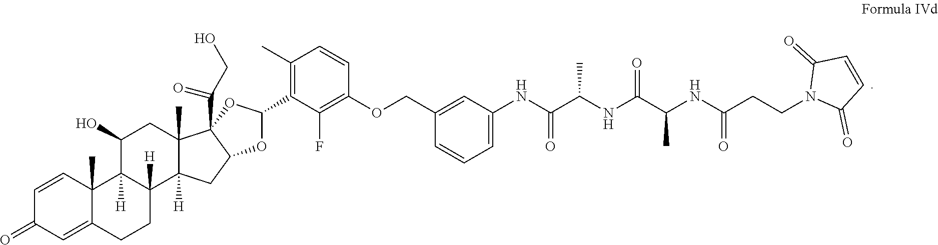

In an embodiment, the disclosure provides a compound of Formula V:

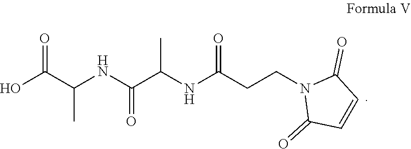 [SMILES: CC(NC(=O)C(C)NC(=O)CCN1C(=O)C=CC1=O)C(=O)O]

In a further embodiment, the disclosure provides a compound of Formula Va:

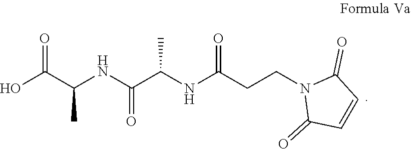 [SMILES: C[C@H](NC(=O)[C@H](C)NC(=O)CCN1C(=O)C=CC1=O)C(=O)O]

In an embodiment, the present disclosure also provides a method of treating an autoimmune disease in a subject in need thereof, comprising administering to the subject an effective amount of a conjugate of Formula I, or a pharmaceutically acceptable salt thereof. In an embodiment, the present disclosure also provides a method of treating an inflammatory disease in a subject in need thereof, comprising administering to the subject an effective amount of a conjugate of Formula I, or a pharmaceutical or salt thereof. In certain embodiments, the autoimmune disease or inflammatory disease is for example, Rheumatoid Arthritis (RA), Psoriatic Arthritis (PsA), Crohn's Disease (CD), Ulcerative Colitis, Plaque Psoriasis (PS), Ankylosing Spondylitis (AS), Juvenile Idiopathic Arthritis, Hidradenitis Suppurativa, Uveitis, Non-Infectious Intermediate, Posterior, Pan Uveitis, Behcet's Disease or Polymyalgia Rheumatica (PMR). In an embodiment, the present disclosure further provides a method of treating rheumatoid arthritis in a subject in need thereof, comprising administering to the subject an effective amount of a conjugate of Formula I, or a pharmaceutically acceptable salt thereof. In an embodiment, the present disclosure further provides a method of treating psoriatic arthritis in a subject in need thereof, comprising administering to the subject an effective amount of a conjugate of Formula I, or a pharmaceutically acceptable salt thereof. In an embodiment, the present disclosure further provides a method of treating Crohn's disease in a subject in need thereof, comprising administering to the subject an effective amount of a conjugate of Formula I, or a pharmaceutically acceptable salt thereof. In an embodiment, the present disclosure further provides a method of treating ulcerative colitis in a subject in need thereof, comprising administering to the subject an effective amount of a conjugate of Formula I, or a pharmaceutically acceptable salt thereof. In an embodiment, the present disclosure further provides a method of treating plaque psoriasis in a subject in need thereof, comprising administering to the subject an effective amount of a conjugate of Formula I, or a pharmaceutically acceptable salt thereof. In an embodiment, the present disclosure further provides a method of treating ankylosing spondylitis in a subject in need thereof, comprising administering to the subject an effective amount of a conjugate of Formula I, or a pharmaceutically acceptable salt thereof. In some embodiments, the subject being administered the effective amount of a conjugate of Formula I, or a pharmaceutically acceptable salt thereof received prior treatment with other anti-TNFα therapeutic, and wherein the subject developed anti-drug antibodies against the other anti-TNFα therapeutic. In such embodiments, the other anti-TNFα therapeutic is selected from the group consisting of adalimumab, infliximab, golimumab, certolizumab or a conjugate thereof. In yet further embodiments, certain conjugates of Formula I, or a pharmaceutically acceptable salt thereof as disclosed herein has low to no cross-reactivity to anti-drug antibodies against at least four or more of other anti-TNFα therapeutic selected from the group consisting of adalimumab, infliximab, golimumab, certolizumab, or a conjugate thereof. In yet further embodiments, certain conjugates of Formula I, or a pharmaceutically acceptable salt thereof as disclosed herein has low to no cross-reactivity to anti-drug antibodies against at least three or more of other anti-TNFα therapeutic selected from the group consisting of adalimumab, infliximab, golimumab, certolizumab, or a conjugate thereof. In yet further embodiments, certain conjugates of Formula I, or a pharmaceutically acceptable salt thereof as disclosed herein has low to no cross-reactivity to anti-drug antibodies against at least two or more of other anti-TNFα therapeutic selected from the group consisting of adalimumab, infliximab, golimumab, certolizumab, or a conjugate thereof. In yet other embodiments, certain conjugates of Formula I, or a pharmaceutically acceptable salt thereof as disclosed herein has low to no cross-reactivity to anti-drug antibodies against adalimumab or a conjugate thereof.

In an embodiment, the present disclosure further provides a conjugate of Formula I, or a pharmaceutically acceptable salt thereof for use in therapy. In an embodiment, the present disclosure provides a conjugate of Formula I, or a pharmaceutically acceptable salt thereof for use in the treatment of an autoimmune disease. In an embodiment, the present disclosure provides a conjugate of Formula I, or a pharmaceutically acceptable salt thereof for use in the treatment of an inflammatory disease. In certain embodiments, the autoimmune disease or inflammatory disease is for example, Rheumatoid Arthritis (RA), Psoriatic Arthritis (PsA), Crohn's Disease (CD), Ulcerative Colitis, Plaque Psoriasis (PS), Ankylosing Spondylitis (AS), Juvenile Idiopathic Arthritis, Hidradenitis Suppurativa, Uveitis, Non-Infectious Intermediate, Posterior, Pan Uveitis, Behcet's Disease or Polymyalgia Rheumatica (PMR). In an embodiment, the present disclosure provides a conjugate of Formula I, or a pharmaceutically acceptable salt thereof, for use in the treatment of rheumatoid arthritis. In an embodiment, the present disclosure provides a conjugate of Formula I, or a pharmaceutically acceptable salt thereof, for use in the treatment of psoriatic arthritis. In an embodiment, the present disclosure provides a conjugate of Formula I, or a pharmaceutically acceptable salt thereof, for use in the treatment of Crohn's disease. In an embodiment, the present disclosure provides a conjugate of Formula I, or a pharmaceutically acceptable salt thereof, for use in the treatment of ulcerative colitis. In an embodiment, the present disclosure provides a conjugate of Formula I, or a pharmaceutically acceptable salt thereof, for use in the treatment of plaque psoriasis. In an embodiment, the present disclosure provides a conjugate of Formula I, or a pharmaceutically acceptable salt thereof, for use in the treatment of ankylosing spondylitis. In some embodiments, the subject being administered the effective amount of a conjugate of Formula I, or a pharmaceutically acceptable salt thereof received prior treatment with other anti-TNFα therapeutic, and wherein the subject developed anti-drug antibodies against the other anti-TNFα therapeutic. In such embodiments, the other anti-TNFα therapeutic is selected from the group consisting of adalimumab, infliximab, golimumab, certolizumab, or a conjugate thereof. In yet further embodiments, certain conjugates of Formula I, or a pharmaceutically acceptable salt thereof as disclosed herein has low to no cross-reactivity to anti-drug antibodies against at least four or more of other anti-TNFα therapeutic selected from the group consisting of adalimumab, infliximab, golimumab, certolizumab, or a conjugate thereof. In yet further embodiments, certain conjugates of Formula I, or a pharmaceutically acceptable salt thereof as disclosed herein has low to no cross-reactivity to anti-drug antibodies against at least three or more of other anti-TNFα therapeutic selected from the group consisting of adalimumab, infliximab, golimumab, certolizumab, or a conjugate thereof. In yet further embodiments, certain conjugates of Formula I, or a pharmaceutically acceptable salt thereof as disclosed herein has low to no cross-reactivity to anti-drug antibodies against at least two or more of other anti-TNFα therapeutic selected from the group consisting of adalimumab, infliximab, golimumab, certolizumab, or a conjugate thereof. In yet other embodiments, certain conjugates of Formula I, or a pharmaceutically acceptable salt thereof as disclosed herein has low to no cross-reactivity to anti-drug antibodies against adalimumab or a conjugate thereof.

In an embodiment, the present disclosure also provides the use of a conjugate of Formula I, or a pharmaceutically acceptable salt thereof, for the manufacture of a medicament for the treatment of an autoimmune disease. In an embodiment, the present disclosure also provides the use of a conjugate of Formula I, or a pharmaceutically acceptable salt thereof, for the manufacture of a medicament for the treatment of an inflammatory disease. In certain embodiments, the autoimmune disease or inflammatory disease is for example, Rheumatoid Arthritis (RA), Psoriatic Arthritis (PsA), Crohn's Disease (CD), Ulcerative Colitis, Plaque Psoriasis (PS), Ankylosing Spondylitis (AS), Juvenile Idiopathic Arthritis, Hidradenitis Suppurativa, Uveitis, Non-Infectious Intermediate, Posterior, Pan Uveitis, Behcet's Disease or Polymyalgia Rheumatica (PMR). In an embodiment, the present disclosure provides the use of a conjugate of Formula I, or a pharmaceutically acceptable salt thereof, for the manufacture of a medicament for the treatment of rheumatoid arthritis. In an embodiment, the present disclosure provides the use of a conjugate of Formula I, or a pharmaceutically acceptable salt thereof, for the manufacture of a medicament for the treatment of psoriatic arthritis. In an embodiment, the present disclosure provides the use of a conjugate of Formula I, or a pharmaceutically acceptable salt thereof, for the manufacture of a medicament for the treatment of Crohn's disease. In an embodiment, the present disclosure provides the use of a conjugate of Formula I, or a pharmaceutically acceptable salt thereof, for the manufacture of a medicament for the treatment of ulcerative colitis. In an embodiment, the present disclosure provides the use of a conjugate of Formula I, or a pharmaceutically acceptable salt thereof, for the manufacture of a medicament for the treatment of plaque psoriasis. In an embodiment, the present disclosure provides the use of a conjugate of Formula I, or a pharmaceutically acceptable salt thereof, for the manufacture of a medicament for the treatment of ankylosing spondylitis. In some embodiments, the subject being administered the effective amount of a conjugate of Formula I, or a pharmaceutically acceptable salt thereof received prior treatment with other anti-TNFα therapeutic, and wherein the subject developed anti-drug antibodies against the other anti-TNFα therapeutic. In such embodiments, the other anti-TNFα therapeutic is selected from the group consisting of adalimumab, infliximab, golimumab, certolizumab or a conjugate thereof. In yet further embodiments, certain conjugates of Formula I, or a pharmaceutically acceptable salt thereof as disclosed herein has low to no cross-reactivity to anti-drug antibodies against at least four or more of other anti-TNFα therapeutic selected from the group consisting of adalimumab, infliximab, golimumab, certolizumab, or a conjugate thereof. In yet further embodiments, certain conjugates of Formula I, or a pharmaceutically acceptable salt thereof as disclosed herein has low to no cross-reactivity to anti-drug antibodies against at least three or more of other anti-TNFα therapeutic selected from the group consisting of adalimumab, infliximab, golimumab, certolizumab, or a conjugate thereof. In yet further embodiments, certain conjugates of Formula I, or a pharmaceutically acceptable salt thereof as disclosed herein has low to no cross-reactivity to anti-drug antibodies against at least two or more of other anti-TNFα therapeutic selected from the group consisting of adalimumab, infliximab, golimumab, certolizumab, or a conjugate thereof. In yet other embodiments, certain conjugates of Formula I, or a pharmaceutically acceptable salt thereof as disclosed herein has low to no cross-reactivity to anti-drug antibodies against adalimumab or a conjugate thereof.

Nucleic acids of the present disclosure may be expressed in a host cell, for example, after the nucleic acids have been operably linked to an expression control sequence. Expression control sequences capable of expression of nucleic acids to which they are operably linked are well known in the art. An expression vector may include a sequence that encodes one or more signal peptides that facilitate secretion of the polypeptide(s) from a host cell. Expression vectors containing a nucleic acid of interest (e.g., a nucleic acid encoding a heavy chain or light chain of an antibody) may be transferred into a host cell by well-known methods, e.g., stable or transient transfection, transformation, transduction or infection. Additionally, expression vectors may contain one or more selection markers, e.g., tetracycline, neomycin, and dihydrofolate reductase, to aide in detection of host cells transformed with the desired nucleic acid sequences.

In another aspect, provided herein are cells, e.g., host cells, comprising the nucleic acids, vectors, or nucleic acid compositions described herein. A host cell may be a cell stably or transiently transfected, transformed, transduced or infected with one or more expression vectors expressing all or a portion of an antibody described herein. In some embodiments, a host cell may be stably or transiently transfected, transformed, transduced or infected with an expression vector expressing HC and LC polypeptides of an antibody of the present disclosure. In some embodiments, a host cell may be stably or transiently transfected, transformed, transduced, or infected with a first vector expressing HC polypeptides and a second vector expressing LC polypeptides of an antibody described herein. Such host cells, e.g., mammalian host cells, can express the antibodies that bind anti-human TNFα as described herein. Mammalian host cells known to be capable of expressing antibodies include CHO cells, HEK293 cells, COS cells, and NS0 cells.

In some embodiments, the cell, e.g., host cell, comprises a vector comprising a first nucleic acid sequence encoding SEQ ID NO: 9, 18, 25, or 35 and a second nucleic acid sequence encoding SEQ ID NO: 10, 19, 28, or 36.

In some embodiments, the cell, e.g., host cell, comprises a first vector comprising a nucleic acid sequence encoding SEQ ID NO: 9, 18, 25, or 35 and a second vector comprising a nucleic acid sequence encoding SEQ ID NO: 10, 19, 28, or 36.

The present disclosure further provides a process for producing an antibody that binds anti-human TNFα described herein by culturing the host cell described above, e.g., a mammalian host cell, under conditions such that the antibody is expressed and recovering the expressed antibody from the culture medium. The culture medium, into which an antibody has been secreted, may be purified by conventional techniques. Various methods of protein purification may be employed, and such methods are known in the art and described, for example, in Deutscher, Methods in Enzymology 182:83-89 (1990) and Scopes, Protein Purification: Principles and Practice, 3rd Edition, Springer, NY (1994).

The present disclosure provides a method of producing a conjugate, the method comprising contacting a compound of the present disclosure with an anti-human TNFα antibody.

The present disclosure provides a method of producing a conjugate, the method comprising contacting the compound of Formula IV with an anti-human TNFα antibody. The present disclosure provides a method of producing a conjugate, the method comprising contacting the compound of Formula IVa with an anti-human TNFα antibody. The present disclosure provides a method of producing a conjugate, the method comprising contacting the compound Formula IVb with an anti-human TNFα antibody. The present disclosure provides a method of producing a conjugate, the method comprising contacting the compound Formula IVc with an anti-human TNFα antibody. The present disclosure provides a method of producing a conjugate, the method comprising contacting the compound Formula IVd with an anti-human TNFα antibody. In some embodiments, the conjugate being produced is the conjugate of Formula I.

The present disclosure provides a method of producing a conjugate, the method comprising the following steps:
(a) reducing an anti-human TNFα antibody with a reducing agent, wherein the anti-human TNFα antibody comprises one or more engineered cysteine residue;
(b) oxidizing the anti-human TNFα antibody with an oxidizing reagent; and
(c) contacting a compound of the present disclosure with the anti-human TNFα antibody to produce the conjugate.

The present disclosure provides a method of producing a conjugate, the method comprising the following steps:
(a) reducing an anti-human TNFα antibody with a reducing agent, wherein the anti-human TNFα antibody comprises one or more engineered cysteine residue;
(b) oxidizing the anti-human TNFα antibody with an oxidizing reagent; and
(c) contacting the compound of the formula

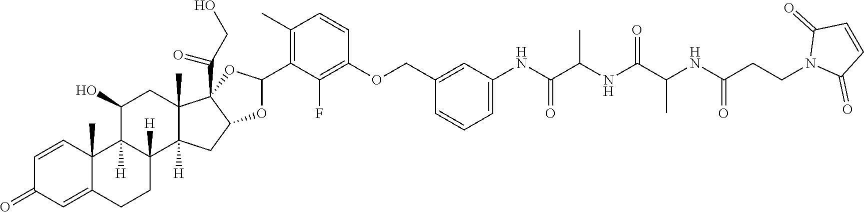

with the anti-human TNFα antibody to produce the conjugate.

In some embodiments, the reducing agent is dithiothreitol. In some embodiments, the oxidizing agent is dehydroascorbic acid. In some embodiments, the reducing agent is dithiothreitol and the oxidizing agent is dehydroascorbic acid.

The present disclosure further provides antibodies or antigen binding fragments thereof produced by any of the processes described herein.

In an embodiment, the present disclosure further provides a pharmaceutical composition, comprising a conjugate of Formula I, or a pharmaceutically acceptable salt thereof, or an antibody, nucleic acid, or vector described herein with one or more pharmaceutically acceptable carriers, diluents, or excipients. In an embodiment, the present disclosure further provides a pharmaceutical composition, comprising a conjugate of Formula I, or a pharmaceutically acceptable salt thereof, with one or more pharmaceutically acceptable carriers, diluents, or excipients. In an embodiment, the present disclosure further provides a pharmaceutical composition, comprising a conjugate of Formula I with one or more pharmaceutically acceptable carriers, diluents, or excipients. In an embodiment, the present disclosure further provides a process for preparing a pharmaceutical composition, comprising admixing a conjugate of Formula I, or a pharmaceutically acceptable salt thereof, with one or more pharmaceutically acceptable carriers, diluents, or excipients. In an embodiment, the present disclosure also encompasses novel intermediates and processes for the synthesis of conjugates of Formula I.

### DETAILED DESCRIPTION OF THE INVENTION

The term “TNFα” as used herein, unless stated otherwise, refers to soluble and/or membrane TNFα, and any native, mature TNFα that results from processing of a TNFα precursor protein in a cell. The term includes TNFα from any vertebrate source, including mammals such as canines, primates (e.g., humans and cynomolgus or rhesus monkeys) and rodents (e.g., mice and rats), unless otherwise indicated. The term also includes naturally occurring variants of TNFα, e.g., splice variants or allelic variants. The amino acid sequence of an example of anti-human TNFα is known in the art, e.g., NCBI accession number: NP_000585 (SEQ ID NO: 39). The amino acid sequence of an example of cynomolgus monkey TNFα is also known in the art, e.g., UniProt reference sequence P79337 (SEQ ID NO: 42). The amino acid sequence of an example of rhesus macaque monkey TNFα is also known in the art, e.g., UniProt reference sequence P48094 (SEQ ID NO: 40). The amino acid sequence of an example of canine TNFα is also known in the art, e.g., GenBank accession number: CAA64403 (SEQ ID NO: 41). The term human “TNFα” is used herein to refer collectively to all known anti-human TNFαisoforms and polymorphic forms. Sequence numbering used herein is based on the mature protein without the signal peptide.

The term “TNFR” or “TNF Receptors” as used herein, unless stated otherwise, refers to any native, mature TNFR e.g., TNFR1 (also known as p55 or p60) or TNFR2 (also known as p75 or p80). The term includes TNFR from any vertebrate source, including mammals such as canines, primates (e.g., humans and cynomolgus or rhesus monkeys) and rodents (e.g., mice and rats), unless otherwise indicated. The term also includes naturally occurring variants of TNFR, e.g., splice variants or allelic variants. The amino acid sequence of an example of human TNFR1 is known in the art, e.g., GenBank accession number: AAA61201 (SEQ ID NO: 45). The amino acid sequence of an example of human TNFR2 is known in the art, e.g., NCBI accession number: NP_001057 (SEQ ID NO: 46). The term “TNFR” is used herein to refer collectively to all known human TNFR isoforms and polymorphic forms.

The term “anti-drug antibodies” or “ADA” as used herein refers to antibodies formed in a mammal from an immune response to a therapeutic administered to that mammal. In some embodiments of the present disclosure, anti-drug antibodies formed against a therapeutic may neutralize the effects of that therapeutic, thus altering the therapeutic's pharmacokinetic (PK) and/or pharmacodynamic (PD) properties, interfering with the effect of the therapeutic, and/or reducing the efficacy, and/or diminishing clinical response to the therapeutic. Anti-drug antibodies to a therapeutic may also lead to adverse immune reaction in a patient such that the patient may not be a candidate for further treatment with that therapeutic. Examples of adverse immune reactions include but are not limited to infusion related reactions characterized by symptoms such as fever, pruritus, bronchospasms, or cardiovascular collapse during or within the first day after drug administration (Atiqi, S., Front Immunol., 2020, 26(11):312).

The term “low to no binding” to anti-drug antibodies as used herein refers to the binding of the anti-human TNFα antibody glucocorticoid conjugates or anti-human TNFα antibodies of the present disclosure to anti-drug antibodies against other anti-TNFα therapeutic wherein such binding is determined to be below the cut-off point of the assay used to measure the binding or is within the pre-determined variability range of the assay. In such methods, the cut-off point is a pre-determined threshold that is used to identify positive binding to anti-drug antibodies. In some embodiments the pre-determined variability of the assay is less than about 20% above the cut-off point of the assay. In such embodiments, binding of the anti-human TNFα Ab GC conjugates or anti-human TNFα antibodies of the present disclosure to anti-drug antibodies against other therapeutic (e.g., adalimumab) that is less than about 20% above the cut-off point of the assay is considered low binding. In some embodiments, binding of the anti-human TNFα Ab GC conjugates or anti-human TNFα antibodies of the present disclosure to anti-drug antibodies against other therapeutic (e.g., adalimumab) that is at or below the cut-off point of the assay is considered no binding.

The term “other anti-TNFα therapeutic” refers to an agent which binds TNFα and inhibits TNF receptor mediated responses, not including the conjugates or anti-human TNFα antibodies described herein. Such an agent may include, but is not limited to, an antibody or a conjugate thereof, antibody fragment or antigen binding fragment, which comprise at least a portion of an antibody retaining the ability to interact with an antigen such as Fab, Fab′, F(ab′)2, Fv fragments, scFv, scFab, disulfide-linked Fvs (sdFv), a Fd fragment or linear antibodies, which may be for example, fused to an Fc region or an IgG heavy chain constant region. In some embodiments, the other anti-TNFα therapeutic may be for example, adalimumab, infliximab, golimumab, and certolizumab, and/or a conjugate thereof.

The term “antibody” as used herein, refers to an immunoglobulin molecule that binds an antigen. Embodiments of an antibody include a monoclonal antibody, polyclonal antibody, human antibody, humanized antibody, chimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4). Embodiments of the present disclosure also include antibody fragments or antigen binding fragments, the term “antibody fragments or antigen binding fragments” comprise at least a portion of an antibody retaining the ability to interact with an antigen such as for example, Fab, Fab′, F(ab′)2, Fv fragments, scFv, scFab, disulfide-linked Fvs (sdFv), a Fd fragment or linear antibodies, which may be for example, fused to an Fc region or an IgG heavy chain constant region.

An exemplary antibody is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. The heavy chain constant region refers to a region of an antibody, which comprises the Fc region and CH1 domain of the antibody heavy chain. Each light chain is comprised of a light chain variable region (VL) and a light chain constant region. The IgG isotype may be further divided into subclasses (e.g., IgG1, IgG2, IgG3, and IgG4). The numbering of the amino acid residues in the constant region is based on the EU index as in Kabat. Kabat et al, Sequences of Proteins of Immunological Interest, 5th edition, Bethesda, MD: U.S. Dept. of Health and Human Services, Public Health Service, National Institutes of Health (1991). The term EU Index numbering or EU numbering is used interchangeably herein.

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987)); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212). A combination of IMGT and North CDR definitions were used for the exemplified anti-human TNFα antibodies as described herein.

The term “Fc region” as used herein, refers to a region of an antibody, which comprises the CH2 and CH3 domains of the antibody heavy chain. Optionally, the Fc region may include a portion of the hinge region or the entire hinge region of the antibody heavy chain. Biological activities such as effector function are attributable to the Fc region, which vary with the antibody isotype. Examples of antibody effector functions include, Fc receptor binding, antibody-dependent cell mediated cytotoxicity (ADCC), antibody-dependent cell mediated phagocytosis (ADCP), C1q binding, complement dependent cytotoxicity (CDC), phagocytosis, down regulation of cell surface receptors (e.g. B cell receptor); and B cell activation.

The term “epitope” as used herein, refers to the amino acid residues of an antigen, that are bound by an antibody. An epitope can be a linear epitope, a conformational epitope, or a hybrid epitope. The term “epitope” may be used in reference to a structural epitope. A structural epitope, according to some embodiments, may be used to describe the region of an antigen which is covered by an antibody (e.g., an antibody's footprint when bound to the antigen). In some embodiments, a structural epitope may describe the amino acid residues of the antigen that are within a specified proximity (e.g., within a specified number of Angstroms) of an amino acid residue of the antibody. The term “epitope” may also be used in reference to a functional epitope. A functional epitope, according to some embodiments, may be used to describe amino acid residues of the antigen that interact with amino acid residues of the antibody in a manner contributing to the binding energy between the antigen and the antibody. An epitope can be determined according to different experimental techniques, also called “epitope mapping techniques.” It is understood that the determination of an epitope may vary based on the different epitope mapping techniques used and may also vary with the different experimental conditions used, e.g., due to the conformational changes or cleavages of the antigen induced by specific experimental conditions. Epitope mapping techniques are known in the art (e.g., Rockberg and Nilvebrant, Epitope Mapping Protocols: Methods in Molecular Biology, Humana Press, 3^{rd} ed. 2018; Holst et al., Molecular Pharmacology 1998, 53(1):166-175), including but not limited to, X-ray crystallography, nuclear magnetic resonance (NMR) spectroscopy, site-directed mutagenesis, species swap mutagenesis, alanine-scanning mutagenesis, steric hindrance mutagenesis, hydrogen-deuterium exchange (HDX), and cross-blocking assays.

The terms “bind” and “binds” as used herein, are intended to mean, unless indicated otherwise, the ability of a protein or molecule to form a chemical bond or attractive interaction with another protein or molecule, which results in proximity of the two proteins or molecules as determined by common methods known in the art.

The terms “nucleic acid” as used herein, refer to polymers of nucleotides, including single-stranded and/or double-stranded nucleotide-containing molecules, such as DNA, cDNA and RNA molecules, incorporating native, modified, and/or analogs of, nucleotides. Polynucleotides of the present disclosure may also include substrates incorporated therein, for example, by DNA or RNA polymerase or a synthetic reaction.

Embodiments of the present disclosure include conjugates where a polypeptide (e.g., anti-human TNFα antibody) is conjugated to one or more drug moieties, such as 2 drug moieties, 3 drug moieties, 4 drug moieties, 5 drug moieties, or more drug moieties. The drug moieties may be conjugated to the polypeptide at one or more sites in the polypeptide, as described herein. In certain embodiments, the conjugates have an average drug-to-antibody ratio (DAR) (molar ratio) in the range of from 2 to 5, or from 3 to 5, or from 3 to 4. In some embodiments, the conjugates have an average DAR of about 3. In certain embodiments, the conjugates have an average DAR of about 4. By average is meant the arithmetic mean.

As used herein, it is understood that the conjugate of Formula I encompasses conjugates of Formulas Ia, Ib, Ic, Id, Ie, and If, and all references to the conjugate of Formula I herein should be read as including conjugates of Formulas Ia, Ib, Ic, Id, Ie, and If. It is further understood by one of skill in the art that the conjugate of Formula I including conjugates of Formulas Ia, Ib, Ic, Id, Ie, and If can also be referred to as anti-human TNFα antibody glucocorticoid conjugates (“anti-human TNFα Ab GC conjugates”).

The anti-human TNFα antibody GC conjugates of the present disclosure can be formulated as pharmaceutical compositions administered by any route which makes the conjugate bioavailable including, for example, intravenous or subcutaneous administration. Such pharmaceutical compositions can be prepared using techniques and methods known in the art (See, e.g., Remington: The Science and Practice of Pharmacy, A. Adej re, Editor, 23^{rd} Edition, published 2020, Elsevier Science).

As used herein, the terms “treating”, “treatment”, or “to treat” includes restraining, slowing, stopping, controlling, delaying, or reversing the progression or severity of an existing symptom or disorder, or ameliorating the existing symptom or disorder, but does not necessarily indicate a total elimination of the existing symptom or disorder. Treatment includes administration of a protein or nucleic acid or vector or composition for treatment of a symptom or disorder in a patient, particularly in a human.

The term “inhibits” or “inhibiting” as used herein, refers to for example, a reduction, lowering, slowing, decreasing, stopping, disrupting, abrogating, antagonizing, or blocking of a biological response or activity, but does not necessarily indicate a total elimination of a biological response.

As used herein, the term “subject” refers to a mammal, including, but are not limited to, a human, chimpanzee, ape, monkey, cattle, horse, sheep, goat, swine, rabbit, dog, cat, rat, mouse, guinea pig, and the like. Preferably the subject is a human.

As used herein, the term “effective amount” refers to the amount or dose of conjugate of the disclosure, or a pharmaceutically acceptable salt thereof which, upon single or multiple dose administration to the subject, provides the desired effect in the subject under diagnosis or treatment. The term “effective amount”, as used herein, further refers to an amount or dose of conjugates of the disclosure, or a pharmaceutically acceptable salt thereof, that will elicit the desired biological or medical response of a subject, for example, reduction or inhibition of a protein activity, or ameliorate symptoms, alleviate conditions, slow or delay disease progression, or prevent a disease, etc. In a non-limiting embodiment, the term “effective amount” refers to the amount necessary (at dosages and for periods of time and for the means of administration) of or dose of conjugate of the disclosure, or a pharmaceutically acceptable salt thereof, when administered to a subject, is effective to at least partially alleviate, inhibit, prevent and/or ameliorate a condition, or a disorder or a disease to achieve the desired therapeutic result. An effective amount is also one in which any toxic or detrimental effects of or dose of conjugate of the disclosure, or a pharmaceutically acceptable salt thereof of the present disclosure are outweighed by the beneficial effects.

An effective amount can be determined by one skilled in the art by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular conjugate administered; the mode of administration; the bioavailability characteristics of the preparation administered; the dose regimen selected; the use of concomitant medication; and other relevant circumstances.

Included within the scope of the present invention is a pharmaceutically acceptable salt of the conjugate of Formula I. A pharmaceutically acceptable salt of a conjugate of the invention, such as a conjugate of Formula I can be formed under standard conditions known in the art. See, for example, Berge, S. M., et al., “Pharmaceutical Salts,” Journal of Pharmaceutical Sciences, 66:1-19, (1977).

TABLE 1
Abbreviations and definitions

| Term | Definition |
|---|---|
| ACN | acetonitrile |
| aq | aqueous |
| cP | centipoise |
| DCM | dichloromethane |
| DIPEA | N,N-diisopropylethylamine |
| DMF | N,N-dimethylformamide |
| DMSO | dimethyl sulfoxide |
| dppf | 1,1'-bis(diphenylphosphino)ferrocene |
| ES/MS | electrospray mass spectrometry |
| EtOAc | ethyl acetate |
| HATU | hexafluorophosphate azabenzotriazole tetramethyl uronium |
| HPLC | high performance liquid chromatography |
| LDA | lithium diisopropylamide |
| MeOH | methanol |
| MS | mass spectrometry |
| MTBE | methyl tert-butyl ether |
| m/z | mass-to-charge ratio |
| NMR | nuclear magnetic resonance |
| Pet ether | petroleum ether |
| rt | room temperature |
| satd | saturated |
| THF | tetrahydrofuran |

The conjugates of the present disclosure, or salts thereof, may be readily prepared by a variety of procedures known to one of ordinary skill in the art, some of which are illustrated in the preparations and examples below. One of ordinary skill in the art recognizes that the specific synthetic steps for each of the routes described may be combined in different ways, or in conjunction with steps from different schemes, to prepare conjugates of the disclosure, or salts thereof. The product of each step can be recovered by conventional methods well known in the art, including extraction, evaporation, precipitation, chromatography, filtration, trituration, and crystallization. All substituents unless otherwise indicated, are as previously defined. The reagents and starting materials are readily available to one of ordinary skill in the art. The following preparations, examples, and assays further illustrate the disclosure, but should not be construed to limit the scope of the disclosure in any way.

### BRIEF DESCRIPTION OF THE DRAWINGS

FIG. 1 shows the in vitro ADCC activity for the exemplified anti-human TNFα Ab1 GC conjugate.

FIG. 2 shows the in vitro CDC activity for the exemplified anti-human TNFα Ab1 GC conjugate.

FIG. 3 shows that exemplified anti-human TNFα antibody Ab6 has significantly low binding to anti-drug antibodies against adalimumab formed in cynomolgus monkeys hyperimmunized with adalimumab.

FIG. 4 shows that exemplified anti-human TNFα antibody Ab6 has significantly low binding to anti-drug antibodies against adalimumab formed in human patients treated with adalimumab.

FIGS. 5A-5C show the DSC thermograms for the exemplified anti-human TNFα Ab1 GC conjugate in PBS, pH 7.2 (5A), Acetate, pH 5 (5B), and Histidine, pH 6 (5C).

FIGS. 6A-6C shows the efficacy comparison of the anti-human TNFα Ab1 GC conjugate, the anti-human TNFα Ab1, and an exemplary anti-human TNFα antibody conjugate in a humanized mouse model of contact hypersensitivity at 1 mg/kg (6A), 3 mg/kg (6B), and 10 mg/kg (6C).

FIG. 7 shows the anti-human TNFα Ab2 GC conjugate in a human TNFα transgenic mouse polyarthritis model arrested disease progression as measured by clinical score in both adalimumab naïve and adalimumab treated mice, showing that the anti-human TNFα Ab2 GC conjugate did not generate a significant anti-drug antibody response, and had low to no cross-reactivity to anti-drug antibodies against adalimumab.

### Preparation 1

### 6-Bromo-2-fluoro-3 -methoxybenzaldehyde

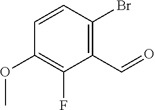

Two reactions were carried out in parallel. To a solution of 4-bromo-2-fluoro-1-methoxybenzene (250 g, 1.2 mol) in THF (1500 mL) was added LDA (2 M, 730 mL) slowly at −78 ° C., over 30 min. After an additional 30 min, DMF (140 mL, 1.8 mol) was added at −78 ° C. slowly over 30 min. After 1 h, the two reactions were combined and the mixture was diluted with aq citric acid (2000 mL) and extracted with EtOAc (1500 mL×2). The combined organic layers were washed with satd aq NaCl (1000 mL) and concentrated under reduced pressure to give a residue. The residue was triturated with petroleum ether (1000 mL) at rt over 12 h to give the title compound (382 g, 67% yield). ES/MS m/z 233.9 (M+H).

### Preparation 2

### 2-Fluoro-3-methoxy-6-methylbenzaldehyde

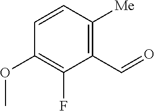

Three reactions were carried out in parallel. 6-Bromo-2-fluoro-3-methoxybenzaldehyde (120 g, 5.3 mol), methylboronic acid (47 g, 7.9 mol), Pd(dppf)Cl₂ (12 g, 0.02 mol), and Cs₂CO₃ (340 g, 1.1 mol) were added to a mixture of 1,4-dioxane (600 mL) and water (120 mL). The mixture was stirred at 120° C. After 12 h, the three reactions were combined and the mixture was diluted with satd aq NH₄Cl (1000 mL) and extracted with MTBE (1500 mL×2). The combined organic layers were washed with satd aq NaCl (1000 mL) and concentrated under reduced pressure to give a residue. The residue was purified by silica gel chromatography, eluting with 40:1 Pet ether:EtOAc to give the title compound (180 g, 59%). ES/MS m/z 169.3 (M+H).

### Preparation 3

### 2-Fluoro-3-hydroxy-6-methylbenzaldehyde

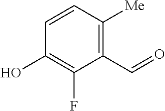 [SMILES: Cc1ccc(O)c(F)c1C=O]

2-Fluoro-3-methoxy-6-methylbenzaldehyde (175 g, 1.0 mol) was added into DCM (1050 mL). BBr₃ (200 mL, 2.1 mol) was added slowly into the solution at 0° C. The reaction was stirred at rt. After 1 h, the mixture was diluted with satd aq NaHCO₃ (1000 mL) until pH=7-8 and then extracted with MTBE (1500 mL×2). The combined organic layers were washed with satd aq NaCl (1000 mL) and concentrated under reduced pressure to give the title compound (110 g, 68%). ES/MS m/z 154.9 (M+H).

### Preparation 4

### tert-Butyl N-[3-[(2-fluoro-3-formyl-4-methyl-phenoxy)methyl]phenyl]carbamate

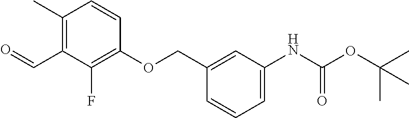 [SMILES: Cc1ccc(OCc2cccc(NC(=O)OC(C)(C)C)c2)c(F)c1C=O]

2-Fluoro-3-hydroxy-6-methylbenzaldehyde (130 g, 0.84 mol), tert-butyl (3-(bromomethyl)phenyl)carbamate (200 g, 0.70 mol), and potassium carbonate (350 g, 2.5 mol) were added in acetonitrile (780 mL) at rt and then heated to 50° C. After 5 h, the reaction was diluted with water (600 mL) and extracted with EtOAc (800 mL×2). The combined organic layers were washed with satd aq NaCl (800 mL) and concentrated under reduced pressure to give a residue. The residue was purified by silica gel chromatography, eluting with 50:1 Pet ether:EtOAc to give the crude product. The crude product was triturated with MTBE (500 mL) at rt for 30 min to give the title compound (103 g, 32%). ES/MS m/z 382.1 (M+Na).

### Preparation 5

### (6aR,6bS,7S,8aS,8bS,10R,11aR,12aS,12bS)-10-(3-((3-Aminobenzyl)oxy)-2-fluoro-6-methylphenyl)-7-hydroxy-8b-(2-hydroxyacetyl)- 6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

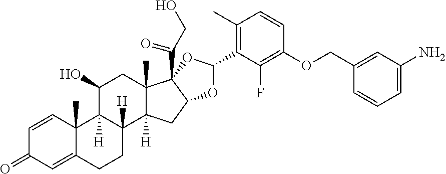

Perchloric acid (70% in water, 4.8 mL) was added to a suspension of (8S,9S,10R,11S,135,14S,16R,17S)-11,16,17-trihydroxy-17-(2-hydroxyacetyl)-10,13-dimethyl-7,8,9,11,12,14,15,16-octahydro-6H-cyclopenta[a]phenanthren- 3-one (4.4 g, 12 mmol, also referred to as “16alpha-hydroxyprednisolone”) and tert-butyl N-[3-[(2-fluoro-3-formyl-4-methyl-phenoxy)methyl]phenyl]carbamate (4.0 g, 11 mmol, preparation 4) in acetonitrile (110 mL) at −10° C. and was warmed to rt. After 1 h, DMF (10 mL) was added to the suspension at rt. After 18 h, the reaction was quenched with satd aq NaHCO₃ and extracted with 9:1 DCM:isopropanol. The organic layers were combined, dried over MgSO₄, filtered, and concentrated under reduced pressure to give a residue. The residue was purified by reverse phase chromatography, eluting with 1:1 aq NH₄HCO₃ (10 mM+5% MeOH):ACN to give the title compound, peak 1 (1.72 g, 25%). ES/MS m/z 618.6 (M+H). ¹H NMR (400.13 MHz, d₆-DMSO) δ0.93-0.87 (m, 6H), 1.40 (s, 3H), 1.71-1.60 (m, 1H), 1.89-1.76 (m, 4H), 2.18-2.12 (m, 2H), 2.29 (s, 4H), 4.23-4.17 (m, 1H), 4.32-4.30 (m, 1H), 4.50-4.43 (m, 1H), 4.81 (d, J=3.2 Hz, 1H), 4.98-4.95 (m, 3H), 5.16-5.10 (m, 3H), 5.61 (s, 1H), 5.95 (s, 1H), 6.18-6.15 (m, 1H), 6.53-6.48 (m, 2H), 6.58 (s, 1H), 6.90-6.86 (m, 1H), 6.99 (t, J=7.7 Hz, 1H), 7.12 (t, J=8.5 Hz, 1H), 7.33-7.30 (m, 1H).

### Preparation 6

### (6aR,6bS,7S,8aS,8bS,10S,11aR,12aS,12bS)-10-(3-((3 -Aminobenzyl)oxy)-2-fluoro-6-methylphenyl)-7-hydroxy-8b-(2- hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4- one (herein also referred to as GC1)

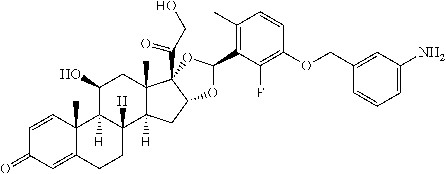

From Preparation 5, the residue was purified by reverse phase chromatography, eluting with 1:1 aq NH₄HCO₃ (10 mM+5% MeOH):ACN to give the title compound, peak 2 (1.24 g, 18%). ES/MS m/z 618.6 (M+H). ¹H NMR (400.13 MHz, d₆-DMSO) d ¹H NMR (400.13 MHz, DMSO): 0.88 (s, 3H), 1.24-1.12 (m, 2H), 1.40 (s, 3H), 1.69-1.56 (m, 1H), 1.91-1.76 (m, 4H), 2.08-2.01 (m, 2H), 2.22 (s, 3H), 2.39-2.29 (m, 1H), 3.18 (d, J=5.2 Hz, 1H), 4.12-4.00 (m, 1H), 4.37-4.30 (m, 2H), 4.79 (d, J=3.1 Hz, 1H), 5.00-4.93 (m, 2H), 5.10-5.06 (m, 3H), 5.31 (d, J=6.7 Hz, 1H), 5.95 (s, 1H), 6.18 (dd, J=1.8, 10.1 Hz, 1H), 6.34 (s, 1H), 6.53-6.48 (m, 2H), 6.58 (s, 1H), 6.87 (d, J=8.5 Hz, 1H), 6.99 (t, J=7.7 Hz, 1H), 7.09 (t, J=8.5 Hz, 1H), 7.33 (d, J=10.1 Hz, 1H).

### Preparation 7

### (3-(2,5-Dioxo-2,5-dihydro-1H-pyrrol-1-yl)propanoyl)-L-alanyl-L-alanine

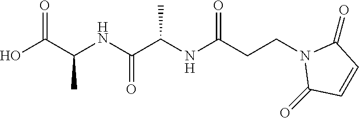 [SMILES: C[C@H](NC(=O)[C@H](C)NC(=O)CCN1C(=O)C=CC1=O)C(=O)O]

To a solution of N-succinimidyl 3-maleimidopropionate (5.0 g, 19 mmol) and L-alanyl-L-alanine (3.4 g, 21 mmol) in DMF (25 mL) was added DIPEA (3.1 mL, 18 mmol) and the mixture was stirred at rt overnight. The reaction mixture was concentrated under reduced pressure to give a residue, which was purified by silica gel chromatography eluting with 2% acetic acid in EtOAc to give the title compound (4.0 g, 69%). ES/MS m/z 312.3 (M+H).

### Preparation 8

### 3-(2,5-Dioxo-2,5-dihydro-1H-pyrrol-1-yl)-N-((S)-1-(((S)-1-((3-((2-fluoro-3-((6aR,6bS,7S,8aS,8bS,10S,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)-4-methylphenoxy)methyl)phenyl)amino)-1-oxopropan-2-yl)amino)-1-oxopropan-2-yl)propanamide (herein also referred to as “GC-L”)

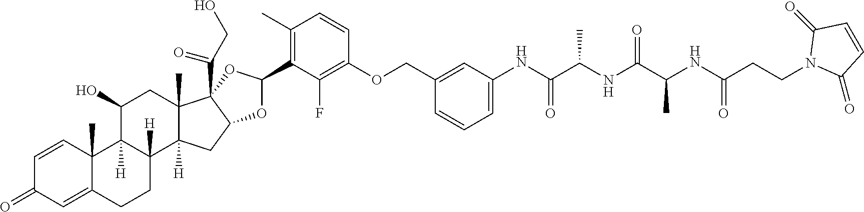

To a solution of (6aR,6bS,7S,8aS,8bS,10S,11aR,12a5,12b5)-10-(3-((3-aminobenzyl)oxy)-2-fluoro-6-methylphenyl)-7-hydroxy-8b- (2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol- 4-one (24 g, 39 mmol, see Preparation 6) and 3-(2,5-dioxo-2,5-dihydro-1H-pyrrol-1-yl)propanoyl)-L-alanyl-L-alanine (15 g, 47 mmol, see Preparation 7) in DMF (250 mL), cooled to 0-5° C., was added 2,6-lutidine (11 mL, 97 mmol) followed by HATU (17 g, 43 mmol). The mixture was stirred at 0-5° C. for 5 min, then the cooling bath was removed, and the mixture was stirred for 2 h. The mixture was diluted with EtOAc. The organic solution was washed with three portions water, one portion satd aq NaCl, dried over Na₂SO₄ (MeOH added to aid solubility), filtered and evaporated to give the crude product. The crude product was purified by silica gel chromatography using a gradient of 1-10% MeOH in DCM to give the title compound (24 g, 68%). ES/MS m/z 911.4 (M+H). ¹H NMR (400.13 MHz, DMSO): d 9.88 (s, 1H), 8.20 (d, J=7.1 Hz, 1H), 8.11 (d, J=7.2 Hz, 1H), 7.68 (s, 1H), 7.60-7.58 (m, 1H), 7.34-7.29 (m, 2H), 7.14-7.09 (m, 2H), 7.00 (s, 2H), 6.89 (d, J=8.4 Hz, 1H), 6.34 (s, 1H), 6.18 (dd, J=1.8, 10.0 Hz, 1H), 5.95 (s, 1H), 5.76 (s, 1H), 5.31 (d, J=6.8 Hz, 1H), 5.13-5.04 (m, 3H), 4.78 (d, J=3.1 Hz, 1H), 4.41-4.30 (m, 4H), 4.10-4.00 (m, 1H), 3.61 (t, J=7.3Hz, 2H), 2.42-2.31 (m, 3H), 2.22 (s, 3H), 2.11-2.01 (m, 2H), 1.91-1.78 (m, 5H), 1.40 (s, 3H), 1.31 (d, J=7.2 Hz, 3H), 1.19-1.11 (m, 5H), 0.88 (s, 3H).

### Preparation 9

### 3-(2,5-dioxo-2,5-dihydro-1H-pyrrol-1-yl)-N-((S)-1-(((S)-1-((3-((2-fluoro-3-((6aR,6bS,7S,8aS,8bS,10R,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxo1-10-yl)-4-methylphenoxy)methyl)phenyl)amino)-1-oxopropan-2-yl)amino)-1-oxopropan-2-yl)propenamide

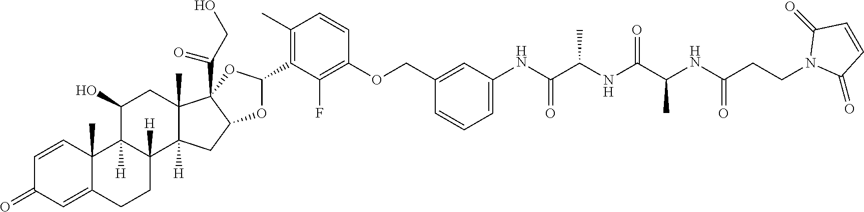

In a manner analogous to the procedure described in Preparation 8, the compound of Preparation 9 was prepared from (6aR,6bS,7S,8aS,8bS,10R,11aR,12aS,12bS)-10-(3-((3-aminobenzyl)oxy)-2-fluoro-6-methylphenyl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one (see Preparation 5) and 3-(2,5-dioxo-2,5-dihydro-1H-pyrrol-1-yl)propanoyl)-L-alanyl-L-alanine (see Preparation 7). ES/MS m/z 911.4 (M+H). 1H NMR (500.11 MHz, DMSO): d 9.88 (s, 1H), 8.23-8.20 (m, 1H), 8.11 (d, J=7.2 Hz, 1H), 7.69 (s, 1H), 7.59 (d, J=8.0 Hz, 1H), 7.33-7.28 (m, 2H), 7.15-7.08 (m, 2H), 7.00 (s, 2H), 6.91-6.89 (m, 1H), 6.17 (dd, J=1.7, 10.1 Hz, 1H), 5.94 (s, 1H), 5.61 (s, 1H), 5.16-5.12 (m, 3H), 4.98-4.96 (m, 1H), 4.81 (d, J=3.1 Hz, 1H), 4.49-4.36 (m, 6H), 3.61 (t, J=7.3 Hz, 2H), 2.41 (t, J=7.3 Hz, 2H), 2.30-2.29 (m, 4H), 2.17-2.15 (m, 2H), 1.88-1.77 (m, 4H), 1.69-1.61 (m, 1H), 1.40 (s, 3H), 1.31 (d, J=7.2 Hz, 3H), 1.18 (d, J=7.2 Hz, 3H), 0.93-0.87 (m, 6H).

### EXAMPLES

### Example 1. Generation of the Anti-Human TNFα Antibody Glucocorticoid Conjugates (Anti-Human TNFα Ab GC Conjugates)

### Example 1a: Generation and Engineering of Anti-Human TNFα Antibodies

Antibody generation: To develop antibodies specific to human TNFα, transgenic mice with human immunoglobulin variable regions were immunized with recombinant human TNFα. Screening was done with human TNFα and the cross reactivity with other TNFα species was tested. Antibodies that are cross reactive to both human and cynomolgus monkey TNFα were cloned, expressed, and purified by standard procedures, and tested for neutralization in a TNFα induced cytotoxicity assay. Antibodies were selected and engineered in their CDRs, variable domain framework regions, and IgG isotype to improve binding affinities and developability properties such as, stability, solubility, viscosity, hydrophobicity, and aggregation.

The amino acid sequence of human TNFα is provided as SEQ ID NO: 39, the amino acid sequence of cynomolgus monkey TNFα is provided as SEQ ID NO: 42.

The anti-human TNFα antibodies can be synthesized and purified by well-known methods. An appropriate host cell, such as Chinese hamster ovarian cells (CHO), can be either transiently or stably transfected with an expression system for secreting antibodies using a predetermined HC:LC vector ratio if two vectors are used, or a single vector system encoding both heavy chain and light chain. Clarified media, into which the antibody has been secreted, can be purified using the commonly used techniques.

Antibody engineering to improve viscosity: The parental TNFα antibody lineage was found to have high viscosity upon concentration. Viscosity is a key developability criteria for assessing feasibility of delivery of a therapeutic antibody via, for example, an autoinjector. Mutagenesis analysis of the antibodies required balancing of improving biophysical properties and retaining desirable affinity and potency without increasing immunogenicity risk. In-silico modeling of the parental antibody was used to identify regions of charge imbalance in the surface comprised of the six complementary determining regions (CDRs). The antibodies generated from the mutagenesis were screened for TNFα binding, and those antibodies which retained or improved target binding as compared to the parental mAb (as determined by ELISA) and had desirable viscosity and other developability properties were selected for further development.

Antibody engineering to reduce the risk of immunogenicity: The anti-human TNFα antibodies were tested in MHC-associated peptide proteomics (MAPPS) assay to determine immunogenicity risk. Briefly, major histocompatibility complex (MHC) bound peptides were identified for antibodies with specific CDR sequences. A CDR library having mutations identified as potentially reducing immunogenicity was constructed and screened for TNFα binding. Antibodies were screened and selected to optimize for low immunogenicity risk whilst balancing maintaining desirable binding affinity to TNFα and other desirable developability properties.

Tables 2a, 2b, and 3 show the exemplified anti-human TNFα antibody sequences optimized for low viscosity, acceptable immunogenicity risk and other desirable developability properties while retaining desirable binding potency to human TNFα.

TABLE 2a
CDR amino acid sequences of exemplified anti-human TNFα Abs

| TNFα | CDR Sequence | | | | | |
|---|---|---|---|---|---|---|
| Antibody | HCDR1 | HCDR2 | HCDR3 | LCDR1 | LCDR2 | LCDR3 |
| Ab1 | SEQ ID | SEQ ID | SEQ ID | SEQ ID | SEQ ID | SEQ ID |
| | NO: 1 | NO: 2 | NO: 3 | NO: 4 | NO: 5 | NO: 6 |
| Ab2 | SEQ ID | SEQ ID | SEQ ID | SEQ ID | SEQ ID | SEQ ID |
| | NO: 1 | NO: 2 | NO: 13 | NO: 14 | NO: 5 | NO: 15 |
| Ab3 | SEQ ID | SEQ ID | SEQ ID | SEQ ID | SEQ ID | SEQ ID |
| | NO: 22 | NO: 23 | NO: 13 | NO: 4 | NO: 5 | NO: 6 |
| Ab4 | SEQ ID | SEQ ID | SEQ ID | SEQ ID | SEQ ID | SEQ ID |
| | NO: 22 | NO: 23 | NO: 13 | NO: 14 | NO: 5 | NO: 15 |
| Ab5 | SEQ ID | SEQ ID | SEQ ID | SEQ ID | SEQ ID | SEQ ID |
| | NO: 22 | NO: 23 | NO: 13 | NO: 14 | NO: 5 | NO: 6 |
| Ab6 | SEQ ID | SEQ ID | SEQ ID | SEQ ID | SEQ ID | SEQ ID |
| | NO: 1 | NO: 2 | NO: 30 | NO: 31 | NO: 5 | NO: 32 |

TABLE 2b
CDR amino acid consensus sequences of exemplified
anti-human TNFα Abs

| Region | Consensus sequence |
|---|---|
| LCDR1 | SEQ ID NO: 43 |
| | QASQGIXaa₇NYLN |
| | Wherein Xaa₇ is Serine or Arginine |
| LCDR3 | SEQ ID NO: 44 |
| | QQYDXaa₅LPLT |
| | Wherein Xaa5 is Asparagine or Lysine |

TABLE 3
Amino Acid sequences of exemplified anti-human TNFα Abs

| TNFα | | | | |
|---|---|---|---|---|
| Anti- | | | | |
| body | VH | VL | HC | LC |
| Ab1 | SEQ ID NO: 7 | SEQ ID NO: 8 | SEQ ID NO: 9 | SEQ ID NO: 10 |
| Ab2 | SEQ ID NO: 16 | SEQ ID NO: 17 | SEQ ID NO: 18 | SEQ ID NO: 19 |
| Ab3 | SEQ ID NO: 24 | SEQ ID NO: 8 | SEQ ID NO: 25 | SEQ ID NO: 10 |
| Ab4 | SEQ ID NO: 24 | SEQ ID NO: 17 | SEQ ID NO: 25 | SEQ ID NO: 19 |
| Ab5 | SEQ ID NO: 24 | SEQ ID NO: 27 | SEQ ID NO: 25 | SEQ ID NO: 28 |
| Ab6 | SEQ ID NO: 33 | SEQ ID NO: 34 | SEQ ID NO: 35 | SEQ ID NO: 36 |

Example 1b. Generation of Anti-Human TNFα Ab1 GC Conjugate wherein n is 4

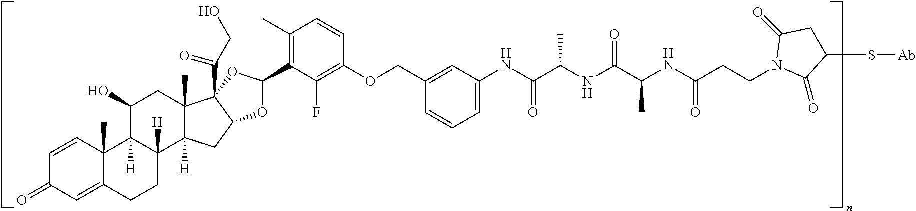

wherein n is 4; and

### Ab is Ab1.

The exemplified anti-human TNFα antibody Ab1 (see Tables 2a, 2b, and 3) was first reduced in the presence of 40-fold molar excess of dithiothreitol (DTT) for 2 hours at 37° C. or>16 hours at ˜21° C. This initial reduction step was used to remove the various capping groups, including cysteine and glutathione which are bound to the engineered cysteine at the 124 and 378 position of the heavy chain during expression. Following the reduction step, the sample was purified through a desalting column to remove the unbound caps as well as the reducing agent. A subsequent 2-hour oxidation step was carried out at room temperature (˜21° C.) in the presence of 10-fold molar excess of dehydroascorbic acid (DHAA) to reform the native interchain disulfides between the light chain and heavy chain as well as the pair of hinge disulfides. After the 2-hour oxidation step, 4-8 molar equivalents of the glucocorticoid receptor agonist payload-linker (“GC-L”), 3-(2,5-dioxo-2,5-dihydro-1H-pyrrol-1-yl)-N-((S)-1-(((S)-1-((3-((2-fluoro-3-((6aR,6bS,7S,8aS,8bS,10S,11aR,12a5,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxo1-10-yl)-4-methylphenoxy)methyl)phenyl)amino)-1-oxopropan-2-yl)amino)-1-oxopropan-2-yl)propanamide prepared in Preparation 8, was added using a 10 mM stock solution solubilized in DMSO. The sample was then incubated at room temperature for 30-60 minutes to allow for efficient conjugation of the GC-L to the engineered cysteines. A subsequent polishing step, such as Size Exclusion Chromatography (SEC) or Tangential Flow Filtration (TFF) was then used to buffer exchange the samples into an appropriate formulation buffer and to remove DMSO and any excess linker-payload.

Drug to antibody ratio (DAR) assessment: To assess the average number of linker-payloads present on the final conjugates, two analytical methods were used: 1) Reverse phase (RP) HPLC and 2) Time of Flight (TOF) mass spectrometry. Both methods required an initial sample reduction step, which included the additional of dithiothreitol (DTT) to a final concentration of ˜10 mM, followed by a 5-minute incubation at 42° C.

Reverse Phase HPLC Method: 10 to 30 μg of the reduced anti-human TNFα antibody Ab1 GC conjugate sample was injected onto a Phenyl SPW, 4.6 mm×7.5 cm, 10 μm olumn (Tosh Part# 0008043). The A buffer was made up of 0.1% trifluoroacetic acid (TFA) in water while B buffer was comprised of 0.1% trifluoroacetic acid (TFA) in acetonitrile (ACN). The column was equilibrated in 20% B buffer prior to sample injection followed by a gradient from 28% B to 40% B over ˜8.5 column volumes. The average DAR was determined by calculating the contribution from each individual DAR species from the fractional percentage multiplied by the DAR number for each contributing species. As this value is based on a partially reduced sample and only represents half of the molecule, the number was then multiplied by 2 to account for an intact antibody GC conjugate. DAR calculations for the anti-human TNFα Ab1 GC conjugate of Example 1b are provided in Table 4.

TABLE 4
Quantification of the average DAR for anti-human TNFα Ab1 GC
conjugate using fractional percentages for each DAR species from a
partially reduced sample.

| DAR | Peak % | DAR Contribution |
|---|---|---|
| 0 | 23.97 | 0.00 |
| 1 | 3.573 | 0.13 |
| Total LC % | 27.543 | |
| | LC Avg DAR | 0.13 |
| | | (DAR contribution from LC) |
| 0 | 0 | 0.00 |
| 1 | 16.059 | 0.22 |
| 2 | 42.631 | 1.18 |
| 3 | 12.525 | 0.52 |
| 4 | 1.242 | 0.07 |
| Total HC % | 72.457 | |
| | HC Avg DAR | 1.99 |
| | | (DAR contribution from HC) |
| (HC + LC)2 | Final Avg DAR | 4.23 |

Time of Flight Mass Spectrometry Method: 8 μg of the partially reduced sample was injected onto a Poroshell 300sb-C3 2.1×2.5 mm, 5 μM column (Agilent Part# 821075-924). Buffer A was made up of 0.1% trifluoroacetic acid (TFA) in water while buffer B comprised of 0.1% trifluoroacetic acid (TFA) in acetonitrile (ACN). The column was equilibrated in 0% B buffer prior to sample injection followed by a gradient from 10% B to 80% B over ˜28 column volumes. The average DAR was determined by calculating the contribution from each individual DAR species from the fractional percentage multiplied by the DAR number for each contributing species. As this value is based on a partially reduced sample and only represents half of the molecule, the number was then multiplied by 2 to account for an intact antibody GC conjugate. DAR calculations for the anti-human TNFα Ab1 GC conjugate of Example 1b are provided in Table 5.

TABLE 5
Quantification of the average DAR for anti-human TNFα Ab1 GC
conjugate using fractional percentages based on total ion counts from
Time of Flight mass spectrometry analysis.

| DAR | Ion counts | DAR Contribution |
|---|---|---|
| 0 | 70408.48 | 0.00 |
| 1 | 748.98 | 0.01 |
| Total LC % | 71157.46 | |
| | LC Avg DAR | 0.01 |
| | | (DAR contribution from LC) |
| 0 | 0 | 0.00 |
| 1 | 11453.18 | 0.14 |
| 2 | 59493.17 | 1.46 |
| 3 | 9902.22 | 0.37 |
| 4 | 424.72 | 0.02 |
| Total HC % | 81273.29 | |
| | HC Avg DAR | 1.99 |
| | | (DAR contribution from HC) |
| (HC + LC)2 | Final Avg DAR | 4.00 |

Example 1c. Generation of Anti-Human TNFα Ab1 GC Conjugate wherein n is 3

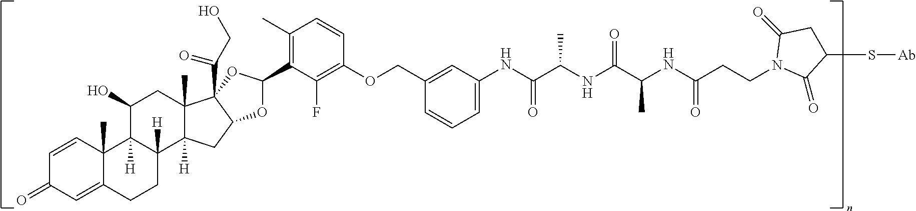

wherein n is 3; and

### Ab is Ab1.

The conjugate of Example 1c was prepared in a manner analogous to the procedure described in Example 1b using a lower molar ratio of the GC-L, 3-(2,5-dioxo-2,5-dihydro-1H-pyrrol-1-yl)-N-((S)-1-(((S)-1-((3-((2-fluoro-3-((6aR,6bS,7S,8aS,8bS,10S,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxo1-10-yl)-4-methylphenoxy)methyl)phenyl)amino)-1-oxopropan-2-yl)amino)-1-oxopropan-2-yl)propanamide to Ab1 during the conjugation step. For example, use of a molar ratio of the corresponding GC-L:Ab1 of 3.2:1 resulted in a final DAR of approximately 3.

Example 1d. Generation of Anti-Human TNFα Ab2 GC Conjugate wherein n is 4

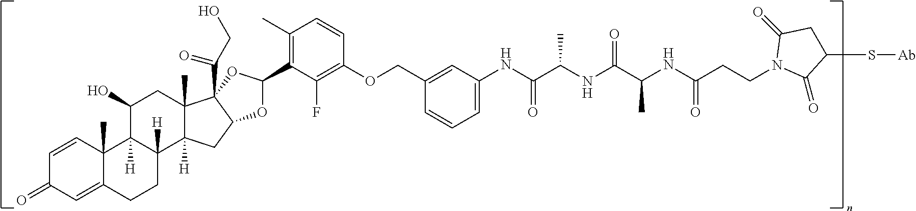

wherein n is 4; and

### Ab is Ab2.

The conjugate of Example 1d was prepared essentially in a manner analogous to the procedure described in Example 1b using Ab2 in place of Ab1.

Example 1e. Generation of Anti-Human TNFα Ab2 GC Conjugate wherein n is 3

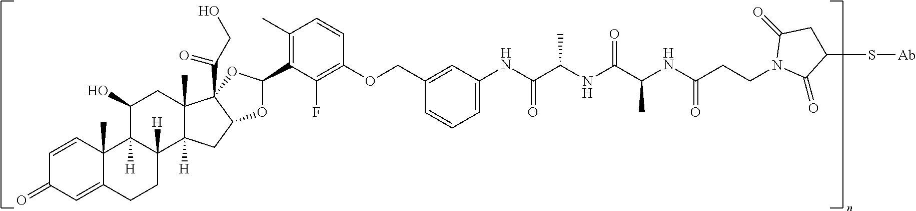

wherein n is 3; and

### Ab is Ab2.

The conjugate of Example le was prepared essentially in a manner analogous to the procedure described in Example lc using Ab2 in place of Ab1.

Example 1f. Thiosuccinimide Hydrolysis: The thiosuccinimide ring of the conjugate Formula Ie wherein n is 4, can be hydrolyzed under conditions well known in the art as shown in Scheme 2 below (See, e.g., WO 2017/210471, paragraph 001226) to provide the ring-opened product of Formula If.

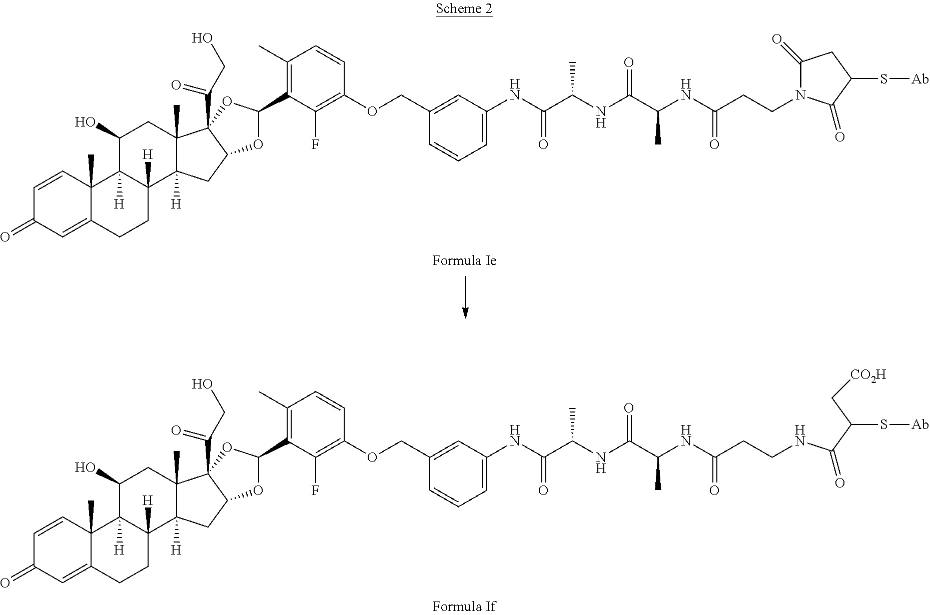

In addition, the above thiosuccinimide ring of the conjugate of Formula Ie may undergo at least partial hydrolysis in vivo and under standard or well known formulation conditions to provide the ring-opened product of Formula If.

### Example 2. Binding Potency of the Anti-Human TNFα Ab1 GC Conjugates and Anti-Human TNFα Antibodies

Example 2a. Elisa Binding: Binding potency of the exemplified anti-human TNFα Ab1 GC conjugate of Example 1b and anti-human TNFα antibodies to human, rhesus macaque, and/or canine TNFα protein was tested in an antigen-down ELISA format. Briefly, 384-well high binding plates (Greiner Bio-one #781061) were coated with 20 μL per well of 1 μg/mL of human TNFα (Syngene), 2 μg/mL of rhesus macaque TNFα (R&D Systems, Cat# 1070-RM), or 2 μg/mL of canine TNFα (R&D Systems, Cat# 1507-CT/CF) diluted in carbonate buffer pH 9.3 (0.015 M Na₂CO₃ and 0.035 M NaHCO₃) and stored at 4° C. overnight. Next day, plates were blocked with 80 μL casein (Thermo Fisher Pierce, Cat# 37528) for 1 h at room temperature, blocking buffer was removed, and 20 μL of titrated anti-human TNFα antibodies expressed in CHO cells and the anti-human TNFα Ab1 GC conjugate (starting concentration at 20 μg/mL diluted in casein and titrated 3-fold, 8 points down) were added to the plates. The plates were incubated at 37° C. for 90 min, then washed three times in PBS/0.1% Tween. 20 μL of secondary antibody reagent goat-anti-human-kappa-AP (Southern Biotech, Cat# 2060-04) with 1:1500 dilution was added to the plate and incubated for 45 minutes at 37° C. Plates were washed 3 times as above, and 20 μL of alkaline phosphatase substrate solution diluted to 1:35 in molecular grade water was added to every well. Once the color developed (approximately 15-30 min), plates were read at 560 nM OD on a Molecular Device Spectramax plate reader and data was acquired using Softmax Pro 4.7 software. Data analysis was performed in GraphPad Prism.

The results in Table 6a show that exemplified anti-human TNFα Ab1 GC conjugate of Example 1b binds human TNFα with desirable potency, that is comparable to the unconjugated anti-human TNFα Ab1.

Representative results in Table 6b, show that the anti-human TNFα antibodies Ab1, Ab2, Ab3, Ab4, Abs, and Ab6 cross-react to human, rhesus macaque monkey, and canine TNFα.

TABLE 6a
Binding EC₅₀ of exemplified anti-human TNFα Ab1 GC conjugate of
Example 1b to human TNFα

| | Human TNFα |
|---|---|
| | EC₅₀ (ug/mL) |
| Ab1 | 0.280 |
| Ab1 GC conjugate | 0.325 |

TABLE 6b
Binding EC₅₀ of exemplified anti-human TNFα antibodies to human,
rhesus macaque, and canine TNFα

| | Human TNFα | Rhesus Macaque | Canine TNFα |
|---|---|---|---|
| | EC₅₀ (μg/mL) | TNFα EC₅₀ (μg/mL) | EC₅₀ (μg/mL) |
| Ab1 | 0.223 | 0.156 | 0.220 |
| Ab2 | 0.222 | 0.162 | 0.24 |
| Ab3 | 0.216 | 0.141 | 0.240 |
| Ab4 | 0.213 | 0.144 | 0.231 |
| Ab5 | 0.220 | 0.161 | 0.176 |
| Ab6 | 0.131 | 0.115 | 0.162 |

Example 2b. Cell surface binding: To evaluate the binding of the exemplified anti-human TNFα Ab1 GC conjugate of Example 1b to live membrane TNFα-expressing cells, known cleavage sites of TNFα were inactivated using a set of mutations previously demonstrated to allow expression of bioactive TNFα on cell surface (Mueller et. al. 1999) in the absence of TNFα cleavage. The non-cleavable TNFα construct was stably transfected into Chinese hamster ovary (CHO) cells. These cells express membrane bound TNFα as shown by flow cytometry.

The TNFα transfected CHO cells were incubated with the exemplified anti-human TNFα Ab1 GC conjugate at concentrations ranging from 600 nM to 0.0304 nM (with three-fold dilution) for 30 minutes at 4° C. in FACS buffer (PBS with 2% FBS). Cell binding was demonstrated by secondary detection with goat anti-human IgG F(ab′)₂, labeled with AlexaFluor-647 (Thermo #A20186) according to the manufacturer protocol. Transfected CHO cells incubated with the exemplified anti-human TNFα Ab1 GC conjugate were washed with FACS buffer and then stained with 2 μg/mL of the AlexaFluor-647 labeled goat anti-human IgG F(ab′)₂ for 30 min at 4° C. in FACS buffer. The stained cells were washed, re-suspended in FACS buffer, and analyzed on a BD LSRFortessa Cell Analyzer. Stains were performed in duplicate. Human IgG1 isotype control antibody was used as a negative control. Anti-human TNFα Ab1 was used as a positive control.

Flow cytometry data was analyzed using FlowJo (v10.8.0) to obtain the mean fluorescence intensity (MFI) of AlexaFluor-647 for each test sample. ECso values were obtained by fitting a non-linear regression (4PL curve) onto the plotted MFI data using GraphPad Prism 9.

The results in Table 7, show that the conjugation of the GC to the anti-human TNFα Ab1 does not significantly affect binding of the anti-human TNFα Ab1 GC conjugate to the membrane expressed TNFα.

TABLE 7
Binding of exemplified anti-human TNFα Ab1 GC conjugate of
Example 1b to membrane human TNFα

| | Binding to membrane human TNFα |
|---|---|
| | EC₅₀ (nM) |
| hIgG1 Isotype Control | n/a |
| Ab1 | 4.258 |
| Ab1 GC conjugate | 5.292 |

### Example 3: In vitro Functional Characterization of the Anti-Human TNFα Ab GC Conjugates

Example 3a. Internalization: The ability of the exemplified anti-human TNFα Ab1 GC conjugate of Example 1b and anti-human TNFα antibodies to bind membrane bound TNFα and internalize into human CD14+monocytes derived from dendritic cells (DC) from different healthy human donors was assessed. CD14+monocytes were isolated from periphery blood mononuclear cells (PBMCs), cultured, and differentiated into immature dendritic cells (with IL-4 and GM-CSF), all using standard protocols. To obtain mature DCs, cells were treated with 1 μg/mL LPS (lipopolysaccharide) for 4 hours.

Exemplified anti-human TNFα Ab1 GC conjugate and anti-human TNFα antibodies were diluted at 8 μg/mL in complete RPMI medium and mixed at equal volume with detection probe Fab-TAMRA-QSY7 diluted to 5.33 μg/mL in complete RPMI medium, then incubated for 30 min at 4° C. in the dark for complex formation, then added to immature and mature DC cultures and incubated for 24 h at 37° C. in a CO₂ incubator. Cells were washed with 2% FBS PBS and resuspended in 100 μL 2% FBS PBS with Cytox Green live/dead dye. Data was collected on a BD LSR Fortessa X-20 and analyzed in FlowJo. Live single cells were gated, and percent of TAMRA fluorescence positive cells was recorded as the readout. To allow the comparison of molecules with data generated from different donors, a normalized internalization index was used. The internalization signal was normalized to IgG1 isotype (normalized internalization index=0) and an internal positive control PC (normalized internalization index=100) using the calculation:

$$100 \times \frac{{X}_{TAMRA} - IgG 1 {isotype}_{TAMRA}}{{PC}_{TAMRA} - IgG 1 {isotype}_{TAMRA}} \qquad ( 1 )$$

where X_{TAMRA}, IgG1 isotype_{TAMRA}, and PC_{TAMRA} were the percent of TAMRA-positive population for the test molecule X, IgG1 isotype, and PC respectively.

The results in Table 8a, show that the anti-human TNFα Ab1 GC conjugate internalized into dendritic cells in vitro upon binding to TNFα expressed on the mature dendritic cells with a comparable internalization index to that of the unconjugated anti-human TNFα Ab1. This indicates that the conjugation of the glucocorticoid to the anti-human TNFα Ab1 does not impact the internalization function of the anti-human TNFα Ab1.

Representative results from a different donor in Table 8b, show that the anti-human TNFα antibodies Ab1, Ab2, Ab3, Ab4, Ab5, and Ab6, internalize into immature and mature dendritic cells upon binding to TNFα on the cell surface.

TABLE 8a
Internalization of exemplified anti-human TNFα Ab1 GC conjugate of
Example 1b into dendritic cells

| | Mature Dendritic Cell |
|---|---|
| | Internalization Index |
| Control hIgG1 | 0 |
| Ab1 | 44.5 |
| Ab1 GC conjugate | 39.5 |

TABLE 8b
Internalization of exemplified anti-human TNFα antibodies
into dendritic cells

| | Immature Dendritic Cell | Mature Dendritic Cell |
|---|---|---|
| | Internalization Index | Internalization Index |
| Ab1 | 19.4 | 26.7 |
| Ab2 | 21.9 | 38.3 |
| Ab3 | 20.0 | 35.2 |
| Ab4 | 33.6 | 43.1 |
| Ab5 | 54.6 | 52.7 |

Example 3b. Inhibition of soluble and membrane TNFα induced apoptosis: Inhibition of soluble and membrane TNFα induced apoptosis by the exemplified anti-human TNFα Ab1 GC conjugate of Example 1b and anti-human TNFα antibodies was evaluated in in vitro cell based assays.

Inhibition of soluble TNFα induced apoptosis: The ability of the exemplified anti-human TNFα Ab1 GC conjugate and the anti-human TNFα antibodies to inhibit soluble TNFα-induced L929 apoptosis assay was evaluated in vitro. L929 mouse fibrosarcoma cells naturally express the TNF receptor. When combined with Actinomycin-D, TNFα induces classical apoptosis in these cells, resulting in rapid cell death due to excessive formation of reactive oxygen intermediates which can be rescued by TNFα neutralization. Briefly, L929 were cultured in assay medium (1× DMEM media, 10% FBS, 1% Pen-Strep, 1% MEM essential amino acids, 1% L-glutamine, 1% sodium pyruvate). On the day of the assay, the cells were rinsed with 1× PBS (no Ca⁺⁺ or Mg⁺⁺) and detached from culture flasks with 0.25% trypsin+EDTA. The trypsin was inactivated with assay medium. L929 cells were centrifuged at 1500 rpm for 5 minutes at room temperature. The cell pellet was resuspended in assay medium, and 1×10⁴ L929 cells (in 100 μL) were added to 96-well plates and placed in a tissue culture incubator (37° C., 95% relative humidity, 5% CO₂) overnight. The conjugate/TNFα/actinomycin-D mixture was (TNFα Ab1 GC conjugate and antibodies were added at 15 μg/mL to 0.0005 μg/mL with three-fold dilution) then transferred to the 96 well plates with L929 adherent cells and incubated (37° C., 95% relative humidity, 5% CO₂) for 18 hours.

To determine number of viable cells, assay medium was removed, and MTS-tetrazolium substrate mixture was added to the wells (100 μL) (where the mitochondrial dehydrogenase enzymes in metabolically active cells reduces the MTS-tetrazolium into a colored formazan product). The plates were placed in an incubator (37° C., 95% relative humidity, 5% CO₂) for 2 hours. The cell death was determined by reading the plates at 490 nm on a microplate reader (Biotek Cytation 5 Imaging Multi-Mode Reader). Results were expressed at the concentration where 50% of the TNFα induced cytotoxicity was inhibited (IC50) by the exemplified anti-human TNFα Ab1 GC conjugate or the antibodies, calculated using a 4-parameter sigmoidal fit of the data (GraphPad Prism 9).

Inhibition of membrane TNFα induced apoptosis: To evaluate the ability of the exemplified anti-human TNFα Ab1 GC conjugate of Example 1b and anti-human TNFα antibodies to inhibit membrane TNFα induced apoptosis, the non-cleavable TNFα construct was stably transfected into Chinese hamster ovary (CHO) cells to generate cell surface (membrane) TNFα expressing CHO cells. The non-cleavable TNFα construct was generated with known mutations at the cleavage sites of the TNFα which allowed for expression of bioactive TNFα on the cell surface (Mueller et. al. 1999) in the absence of TNFα cleavage. Incubation of L929 cells with CHO cells expressing the human non-cleavable TNFα resulted in rapid L929 cell death. To determine whether the exemplified anti-human TNFα Ab1 GC conjugate and anti-human TNFα antibodies could neutralize the observed apoptosis a dose range from 15 μg/mL to 0.0005 μg/mL (with three-fold dilution) was evaluated. Each concentration of the exemplified anti-human TNFα Ab1 GC conjugate or anti-human TNFα antibodies was added at 100 μL/well in duplicate to plates containing 500 CHO TNFα transfectant cells/well+6.25 μg/mL actinomycin-D. The mixtures were incubated for 30 min at room temperature and then added into the L929 cell plate. Human IgG1 isotype control antibody was used as a negative control. The L929 cell death was determined essentially as described for the soluble TNFα induced apoptosis assay.

The results in Table 9a, show that the exemplified anti-human TNFα Ab1 GC conjugate of Example 1b inhibited soluble human TNFα (IC50 of about 0.104 μg/mL) and membrane human TNFα (IC 50 of about 0.306 μg/mL) induced apoptosis of L929 cells in a dose dependent manner in vitro, comparable to the anti-human TNFα Ab1. This indicates that conjugation of the GC to the antibody does not affect the biological activity of the antibody. The negative control hIgG1 isotype as expected, did not inhibit TNFα induced apoptosis.

Representative results in Table 9b, show that the exemplified anti-human TNFα antibodies Ab1, Ab2, Ab3, Ab4, Abs, and Ab6 inhibited both soluble and membrane human TNFα induced apoptosis of L929 cells in a dose dependent manner in vitro.

TABLE 9a
Exemplified anti-human TNFα Ab1 GC conjugate of Example 1b inhibits
soluble and membrane human TNFa induced apoptosis of L929 cells

| | Inhibition of | Inhibition of |
|---|---|---|
| | soluble human | membrane human |
| | TNFα induced apoptosis | TNFα-induced apoptosis |
| | IC₅₀ (μg/mL) | IC₅₀ (μg/mL) |
| hlgG1 Isotype | n/a | n/a |
| Ab1 | 0.089 | 0.302 |
| Ab1 GC conjugate | 0.104 | 0.306 |

TABLE 9b
Exemplified anti-human TNFα antibodies inhibit membrane and soluble
human TNFα-induced apoptosis of L929 cells

| | Inhibition of soluble human | Inhibition of membrane human |
|---|---|---|
| | TNFα induced apoptosis | TNFα-induced apoptosis |
| | IC₅₀ (μg/mL) | IC₅₀ (μg/mL) |
| hlgG1 Isotype | n/a | n/a |
| Ab1 | 0.16 | 0.13 |
| Ab2 | 0.13 | 0.13 |
| Ab3 | 0.19 | 0.15 |
| Ab4 | 0.19 | 0.13 |
| Ab5 | 0.22 | 0.20 |

Example 3c. In vitro human T cell cytokine expression assay: To evaluate the functional activity of the exemplified anti-human TNFα Ab1 GC conjugate of Example 1b and anti-human TNFα Ab2 GC conjugates Example 1d on disease-relevant cells, human primary T cells were stimulated and co-treated with the conjugates in vitro. Activity of an exemplary anti-human TNFα Ab GC conjugate from US2020338208 was also evaluated. Human primary CD3⁺T cells were isolated from freshly purified human PBMCs by immunomagnetic negative selection, according to the manufacturer's protocol (Human T Cell Isolation Kit, Stemcell Technologies #17951). Flow cytometry staining was used to assess cell purity on a BD LSRFortessa Cell Analyzer. T cells were confirmed CD3⁺ (anti-human CD3-APC, Fisher Scientific #17-0036-42) with additional phenotyping for CD4 (anti-human CD4-eFluor-450, Fisher Scientific #48-0047-42) and CD8 (anti-human CD8a-FITC, BioLegend #301006) T cell subsets. 2×10⁵ CD3⁺T cells/well were seeded into 96-well flat bottom plates in assay medium (1× RPMI-1640 media, 10% FBS, 1% non-essential amino acids, 1% sodium pyruvate, 1% Glutamax, 1% Pen-Strep, and 0.1% β-mercaptoethenol). Cells were stimulated with 1×10⁵ Human T-Activator anti-CD3/CD28 Dynabeads (Thermo Fisher #11132D) and treated with the each of the exemplified anti-human TNFα Ab1 GC conjugate, anti-human TNFα Ab2 GC conjugate or the exemplary anti-human TNFα Ab GC conjugate from US2020338208 at 200 nM to 0.0914 nM (with 3-fold dilution), in duplicate plates per donor. Human IgG1 isotype control antibody was used as a negative control. Controls included unconjugated anti-human TNFα Ab1 and free GC. Assay plates were then incubated at 37° C. with 5% CO2 for 72 hours. Cell culture supernatants were harvested at 72 hours and frozen at −80° C. Cytokine levels were measured from thawed cell culture supernatants using custom U-PLEX Human Biomarker Multiplex Assays (Mesoscale Discovery #K15067L) with detection antibodies specific for human IL-6, IL-10, IL-13, and GM-CSF. Activity was measured as the inhibition of the cytokines IL-6, IL-13, GM-CSF, and the induction of IL-10. For each individual donor, the detected levels of cytokine (pg/mL) were converted to normalized ‘% inhibition’ or ‘% induction’ values. The normalization parameters for IL-6, IL-13, and GM-CSF set 0% inhibition equaled the average concentration of cytokine in the stimulated-untreated control wells, with 100% inhibition equal to the average concentration of cytokine in the unstimulated control wells. The normalization parameters for IL-10 set 0% induction equal the average concentration of cytokine in the stimulated-untreated control wells, with 100% induction equal to the average concentration of cytokine in the 200 nM free GC treatment group. Normalized IC₅₀ values were obtained by fitting a non-linear regression (4PL curve) onto the normalized data. Statistical analysis was performed using GraphPad Prism 9.

The results in Table 10, show that the anti-human TNFα Ab GC conjugates of Example 1b and Example 1d significantly inhibited cytokines IL-13, IL-6, and GM-CSF, and significantly induced cytokine IL-10. Additionally, the anti-human TNFα Ab GC conjugates of Example 1b and Example 1d inhibited cytokines IL-13, IL-6, and GM-CSF, and induced cytokine IL-10 at a greater percent than the anti-human TNFα Ab1 or exemplary anti-TNFα Ab GC conjugate disclosed in US2020338208 in this in vitro assay. Particularly, the results show that the anti-human TNFα Ab1 GC conjugate of Example 1b and the anti-human TNFα Ab2 GC conjugate Example 1d modulate cytokine expression via both the TNFα antibody and the glucocorticoid.

TABLE 10
Effect of anti-human TNFα Ab1 GC conjugate of Example 1b and anti-
human TNFα Ab2 GC conjugate of Example 1d on T cell cytokine release

| | IL-6 | IL-13 | GM-CSF | IL-10 |
|---|---|---|---|---|
| | % Inhibition | % Inhibition | % Inhibition | % Induction |
| | at 200 nM | at 200 nM | at 200 nM | at 200 nM |
| | (SEM) | (SEM) | (SEM) | (SEM) |
| hIgG1 Isotype | 6.53 (4.70) | 7.32 (4.02) | 8.61 (4.88) | 16.52 (4.75) |
| Ab1 (n = 14) | 32.24 (3.82) * | 28.07 (3.33)* | 25.59 (4.13)* | 42.47 (7.87) |
| Ab1 GC | 56.76 (5.17)*\hat{ } | 58.48 (2.85)* \hat{ } † | 53.94 (3.04)* \hat{ } † | 114.41 (12.37)*\hat{ } |
| conjugate | | | | |
| (n = 12) | | | | |
| Ab2 GC | 48.52 (3.11)* | 60.16 (2.58)* \hat{ } † | 52.94 (2.47)*\hat{ } | 102.27 (8.60)*\hat{ } |
| conjugate | | | | |
| (n = 12) | | | | |
| Exemplary anti- | 41.18 (4.20)* | 42.25 (2.94)*\hat{ } | 38.79 (2.96)* | 87.06 (12.19)*\hat{ } |
| human TNFα | | | | |
| Ab conjugate | | | | |
| from | | | | |
| US2020338208 | | | | |
| (n = 14) | | | | |

Stats: Tukey's multiple comparisons test, * = p < 0.05 compared to isotype, \hat{ } = p < 0.05 compared to Ab1, † = p < 0.05 compared to exemplary anti-human TNFα Ab conjugate from US2020338208.

### Example 4. Effector Function Activity of the Exemplified Anti-Human TNFα Ab GC Conjugates

Example 4a. Human Fcγ receptor binding. The binding affinity of the exemplified anti-human TNFα Ab1 GC conjugate of Example 1b to human Fcγ receptors was evaluated by surface plasmon resonance (SPR) analysis. A series S CM5 chip (Cytiva P/N BR100530) was prepared using the manufacturer's EDC/NHS amine coupling method (Cytiva P/N BR100050). Briefly, the surfaces of all 4 flow cells (FC) were activated by injecting a 1:1 mixture of EDC/NHS for 7 minutes at 10 μL/minute. Protein A (Calbiochem P/N 539202) was diluted to 100 μg/mL in 10 mM acetate, pH 4.5 buffer, and immobilized for approximately 4000 RU onto all 4 FCs by 7-minute injection at a flow rate of 10 μL/minute. Unreacted sites were blocked with a 7-minute injection of ethanolamine at 10 μL/minute. Injections of 2×10 μL of glycine, pH 1.5, was used to remove any noncovalently associated protein. Running buffer was 1× HBS-EP+ (TEKNOVA, P/N H8022). The FcyR extracellular domains (ECDs) —FcγRI (CD64), FcγRIIA_131R, and FcγRIIA_131H (CD32a), FcγRIIIA_158V, FcγRIIIA_158F (CD16a), and FcγRIIb (CD32b) were produced from stable CHO cell expression and purified using IgG Sepharose and size exclusion chromatography. For FcγRI binding, test molecules (which include the anti-human TNFα Ab1 GC conjugate of Example 1b and a human IgG1 isotype control antibody) were diluted to 2.5 μg/mL in running buffer, and approximately 150 RU of each antibody was captured in FCs 2 through 4 (RU captured). FC1 was the reference FC, therefore no antibody was captured in FC1. FcγRI ECD was diluted to 200 nM in running buffer and then two-fold serially diluted in running buffer to 0.78 nM. Duplicate injections of each concentration were injected over all FCs at 40 μL/minute for 120 seconds followed by a 1200 second dissociation phase. Regeneration was performed by injecting 15 μL of 10 mM glycine, pH 1.5, at 30 μL/minute over all FCs. Reference-subtracted data was collected as FC2 FC1, FC3-FC1, and FC4-FC1 and the measurements were obtained at 25° C. The affinity (K_{D}) was calculated using either steady state equilibrium analysis with the Scrubber 2 Biacore Evaluation Software or a “1:1 (Langmuir) binding” model in BIA Evaluation. For FcγRIIa, FcγRIIb, and FcγRIIIa binding, the test molecules were diluted to 5 μg/mL in running buffer, and approximately 500 RU of each antibody was captured in FCs 2 through 4). FC1 was the reference FC. Fcγ receptor ECDs were diluted to 10 μM in running buffer and then serially diluted 2-fold in running buffer to 39 nM. Duplicate injections of each concentration were injected over all FCs at 40 μL/minute for 60 seconds followed by a 120 second dissociation phase. Regeneration was performed by injecting 15 μL of 10 mM glycine, pH 1.5, at 30 μL/minute over all FCs. Reference-subtracted data was collected as FC2-FC1, FC3-FC1, and FC4-FC1, and the measurements were obtained at 25° C. The affinity (K_{D}) was calculated using the steady state equilibrium analysis with the Scrubber 2 Biacore Evaluation Software. Each receptor was assayed at least two times.

The results in Table 11, show that the binding affinities (K_{D}) of the exemplified anti-human TNFα Ab1 GC conjugate of Example 1b to human FcγRI, FcγRIIa, FcγRIIb, and FcγRIIIa receptor ECDs are comparable to the human IgG1 isotype control antibody.

TABLE 11
Binding affinities of exemplified anti-human TNFα Ab1 GC conjugate of
Example 1b to human Fcγ receptors

| | Human IgG1 isotype | | anti-human TNFα | |
|---|---|---|---|---|
| | control antibody | | Ab1 GC conjugate | |
| | Average | | Average | |
| Fcγ Receptor | K_{D} | Std Dev | K_{D} | Std Dev |
| FcγRI | 47.9 pM | 13.1 | 48.6 pM | 12.1 |
| FcγRIIA_131H | 0.57 μM | 0.04 | 0.35 μM | 0.02 |
| FcγRIIA_131R | 0.57 μM | 0.02 | 0.31 μM | 0.01 |
| FcγRIIb | 2.81 μM | 0.14 | 1.30 μM | 0.05 |
| FcγRIIIA_158V | 0.15 μM | 0.00 | 0.12 μM | 0.01 |
| FcγRIIIA_158F | 0.82 μM | 0.01 | 0.52 μM | 0.01 |

Example 4b. Antibody dependent cellular cytotoxicity (ADCC): In vitro ADCC assays of the exemplified anti-human TNFα Ab1 GC conjugate of Example 1b was evaluated with a reporter gene based ADCC assay.

For the reporter gene based ADCC assay, a CHO cell line co-expressing membrane bound TNFα and CD20 (Eli Lilly and Co.) was used as the target cell line and Jurkat cells expressing functional FcγRIIIa (V158)-NFAT-Luc (Eli Lilly and Company) as the effector cell line were used. All test antibodies and cells were diluted in assay medium containing RPMI-1640 (no phenol red) with 0.1 mM non-essential amino acids (NEAA), 1 mM sodium pyruvate, 2 mM L-glutamine, 500 U/mL of penicillin-streptomycin, and 0.1% w/v BSA. Test antibodies were first diluted to a 3× concentration of 20 nM and then serially diluted 7 times in a 1:4 ratio. 50 μL/well of each antibody was aliquoted in triplicate in white opaque bottom 96-well plate (Costar, #3917). CD20 antibody was used as a positive control. Daudi target cells were then added to the plates at 5×10⁴ cells/well in 50 μL aliquots, and incubated for 1 hour at 37° C. Next, Jurkat V158 cells were added to the wells at 1.5×10⁵ cells/well in 50 aliquots and incubated for 4 hours at 37° C., followed by addition of 100 μL/well of One-Glo Luciferase substrate (Promega, #E8130). The contents of the plates were mixed using a plate shaker at low speed, incubated at room temperature for 5 minutes, and the luminescence signal was read on a BioTek microplate reader (BioTek Instruments) using 0.2 cps integration. Data was analyzed using GraphPad Prism 9 and the relative luminescence units (RLU) for each antibody concentration were plotted in a scatter format of antibody concentration versus RLU. Results were representative of two independent experiments.

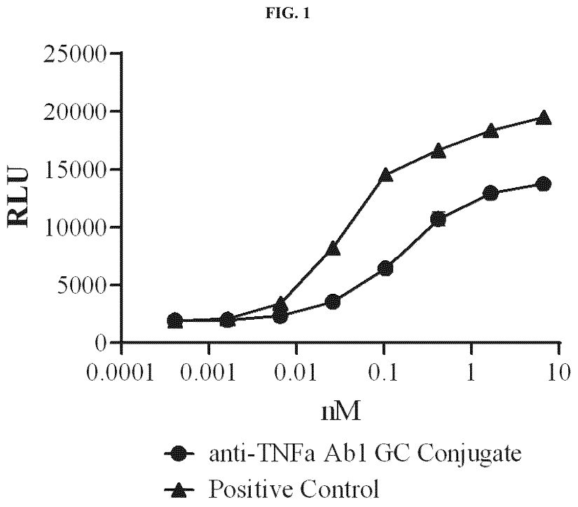

The results in FIG. 1, show that the exemplified anti-human TNFα Ab1 GC conjugate of Example 1b had moderate ADCC activity as compared to the positive control.

Example 4c. Complement dependent cellular cytotoxicity (CDC): In vitro CDC assays of the exemplified anti-human TNFα Ab1 GC conjugate of Example 1b was conducted using a CHO cell line co-expressing membrane bound TNFα and CD20 (Eli Lilly and Co.). All test antibodies, complement, and cells were diluted in assay medium consisting of RPMI-1640 (no phenol red) with 0.1 mM non-essential amino acids (NEAA), 1 mM sodium pyruvate, 2 mM L-glutamine, 500 U/mL of penicillin-streptomycin, and 0.1% w/v BSA. Test antibodies were first diluted to a 3× concentration of 200 nM and then serially diluted 7 times in a 1:4 ratio. 50 μL/well of each antibody (including the CD20 positive control antibody) was aliquoted in triplicate in white opaque bottom 96-well plate (Costar, #3917). Daudi target cells were added at 5×10⁴ cells/well at 50 μL/well and incubated for 1 hour at 37° C. Next, human serum complement (Quidel, #A113) quickly thawed in a 37° C. water bath was diluted 1:6 in assay medium and added at 50 μL/well to the assay plate. The plate was incubated for 2 hours at 37° C., followed by addition of 100 μL/well CellTiter Glo substrate (Promega, #G7571). The contents of the plates were mixed using a plate shaker at low speed, incubated at room temperature for 5 minutes, and the luminescence signal was read on a BioTek microplate reader (BioTek instruments) using 0.2 cps integration. Data was analyzed using GraphPad Prism 9 and the relative luminescence units (RLU) for each antibody concentration were plotted in a scatter format of antibody concentration versus RLU.

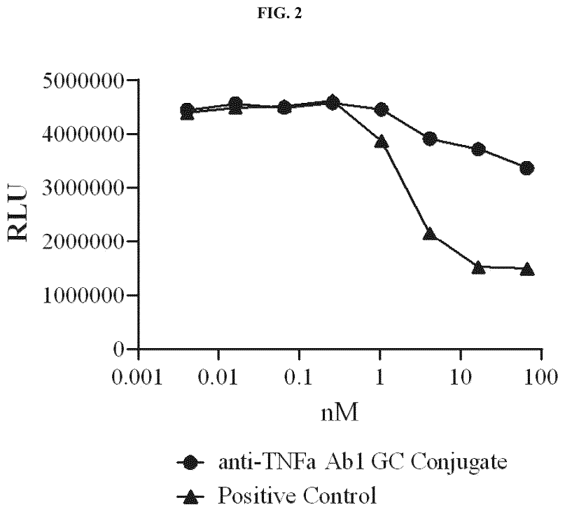

Results of two representative independent experiments (one of which is shown in FIG. 2) showed that the exemplified anti-human TNFα Ab1 GC conjugate had marginal induction of CDC activity when compared to the positive control.

### Example 5: Characterization of Exemplified Anti-Human TNFα Antibody Binding to Anti-Drug Antibodies Against Adalimumab

Example 5a. Binding to Cynomolgus monkey anti-drug antibodies against adalimumab: Binding of exemplified anti-human TNFα antibodies to anti-drug antibodies against adalimumab (anti-adalimumab antibodies) obtained from affinity purified hyperimmune monkey serum (AP-HIMS) from cynomolgus monkeys hyperimmunized with adalimumab was evaluated. An adalimumab-AffiGel10 was used to purify the anti-adalimumab antibodies from the adalimumab hyperimmunized Cynomolgus monkeys. The anti-adalimumab antibodies were detected using a titration of AP-HIMS in an ACE-Bridge assay. The assay was developed following the FDA Guidance on Immunogenicity testing. Briefly, streptavidin-coated 96-well plates (Pierce, 15500) were washed with 1× TBST (Boston BioProducts, IBB-181X), and coated with 30 nM biotinylated adalimumab at 100 μL/well in TB ST/0.1% bovine serum albumin (BSA; Sigma, A7888) for 1 h at room temperature. Plates were washed three times with TBST, and affinity purified anti-adalimumab antibodies were diluted 1:10 with TBS (Fisher, BP2471-1), and added at 100 uL/well to the coated plates and incubated overnight at 4° C. The following day, plates were washed three times with TBST and the captured anti-adalimumab antibodies were acid eluted using 65 μL/well of 300 mM acetic acid (Fisher Scientific, A38-500) for 5 min at room temperature. Polypropylene 96-well plates (Corning, 3359) were then loaded with 50 μL of 1 μg/mL each of biotinylated adalimumab and ruthenium-labeled adalimumab in neutralizing buffer (0.375 M Tris, 300 mM NaCl, pH 9). Next, 50 μL of the acid eluted samples were added to the polypropylene plate containing the mixture in neutralizing buffer and the ADA and were allowed to bridge to the labeled antibodies for 1 h at room temperature. MSD Gold 96-well streptavidin plates (Mesoscale, L15SA-1) were washed and blocked with TBS+1% BSA for 1 h at room temperature, then washed, and 80 μL of bridged samples were added to the plate for 1 h. The plates were washed three times with TBST, and 150 μL/well of 2×MSD Buffer (Mesoscale, R92TC-2) was added to the plates. Plates were read on an MSD SQ120 reader to provide the Tier 1 signal expressed as electro chemiluminescent units (ECLU).

The same AP-HIMS was also tested in the ACE-Bridge assay to detect antibodies against exemplified anti-human TNFα antibody Ab6, following essentially the same method outlined above for adalimumab, but using biotin and ruthenium-labeled Ab6. The resulting ECLU signal was then plotted as a function of the concentration of AP-HIMS tested.

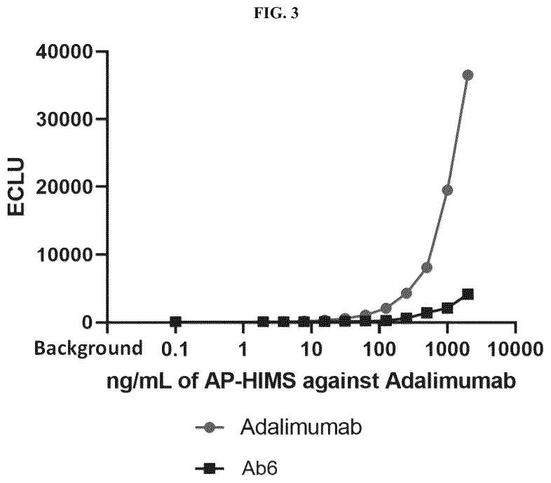

The results in FIG. 3, show that the exemplified anti-human TNFα antibody Ab6 had low to no binding to the anti-drug antibodies against adalimumab (maximum ECLU signal of 4000) purified from serum from Cynomolgus monkey hyperimmunized with adalimumab, when compared to binding of adalimumab to its own anti-drug antibodies (maximum ECLU signal 40000). Specifically, the results showed that the exemplified anti-human TNFα antibody Ab6 only recognized about 10% of the anti-drug antibodies against adalimumab raised by the cynomolgus monkeys suggesting that this binding is likely due to shared sequences located away from the CDR regions, such as the antibody constant region.

Example 5b. Binding to human patient anti-drug antibodies against adalimumab: Binding of exemplified anti-human TNFα antibodies to anti-drug antibodies against adalimumab (anti-adalimumab antibodies) in 21 patient serum samples obtained from adalimumab-treated patients enrolled in the study RA-BEAM was evaluated. The 21 serum samples were collected post-baseline, and were confirmed to have high ADA titers against adalimumab, by using the methods essentially as described for the Cynomolgus monkey ADA evaluation. The 21 serum samples were then evaluated for binding to exemplified anti-human TNFα antibody Ab6 using the methods essentially as described for the Cynomolgus monkey ADA evaluation.

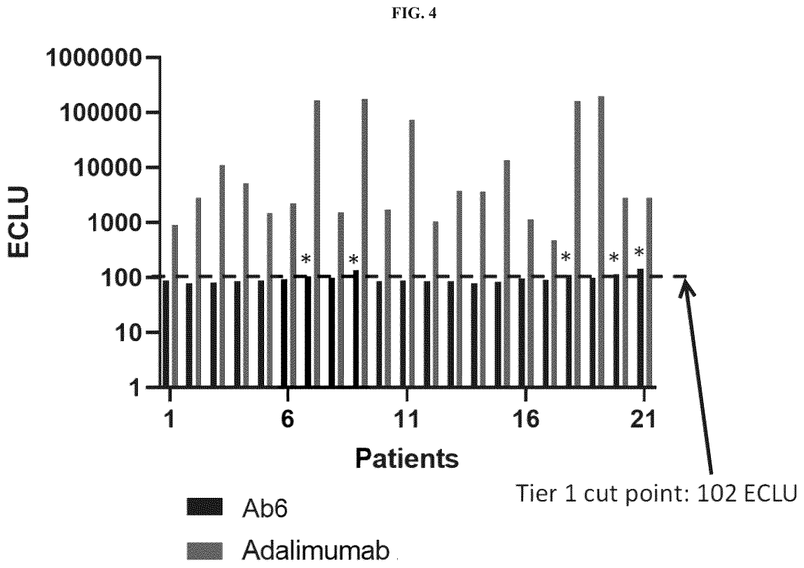

The results in FIG. 4, show that the exemplified anti-human TNFα antibody Ab6 had low to no binding to the anti-drug antibodies against adalimumab in 16 of the 21 patient samples tested (ECLU signal was below the cut-off point of the assay (102 ECLU)). In determining immunogenicity, the cut-off point is a threshold that is used to identify “putative positive”, or anti-drug antibody containing, samples. As shown in FIG. 4, five out of 21 samples had an ECLU signal above the cut-off point, but were all less than 20% above the cut-off point, and therefore determined to be within the variability of the assay. The significantly low or no binding of anti-human TNFα antibody Ab6 to the anti-drug antibodies against adalimumab in human patients treated with adalimumab indicated that the Ab6 and adalimumab antibody sequences are sufficiently different, such that, the ADA raised against adalimumab by the human subjects tested, which are specific to epitopes present in adalimumab, are not shared by Ab6, and thus not significantly recognized by Ab6.

These results indicate potential use of the exemplified anti-human TNFα antibodies or conjugates for treatment of subjects who develop diminished clinical response or adverse reactions to anti-drug antibodies against other TNFα therapeutics such as adalimumab.

### Example 6: Immunogenicity Assessment

Example 6a. MHC-associated peptide proteomics (MAPPs) Assay: MAPPs profiles the MHC-II presented peptides on human dendritic cells previously treated with exemplified anti-human TNFα antibodies. CD14+monocytes were isolated from periphery blood mononuclear cells (PBMCs), cultured and differentiated into immature dendritic cells (with IL-4 and GM-CSF) using standard protocols. Exemplified antibodies were added to the immature dendritic cells on day 4 and fresh media containing LPS to transform the cells into mature dendritic cells was exchanged after 5-hour incubation. The matured dendritic cells were lysed in RIPA buffer with protease inhibitors and DNAse the following day. Immunoprecipitation of MHC-II complexes was performed using biotinylated anti-MHC-II antibody coupled to streptavidin beads. The bound complex was eluted and filtered. The isolated MHC-II peptides were analyzed by a mass spectrometer. Peptide identifications were generated by an internal proteomics pipeline using search algorithms with no enzyme search parameter against a bovine/human database with test sequences appended to the database. Peptides identified from the exemplified antibodies were aligned against the parent sequence.

The results in Table 12, show that the exemplified anti-human TNFα antibodies had varying degree of presentation by MAPPs. Ab1 demonstrated the lowest MAPPs presentation with 1 non-germline cluster in 3 of the 10 donors tested.

TABLE 12
MAPPs analysis of exemplified anti-human TNFα antibodies

| | Number of non-germline | Number of donors containing |
|---|---|---|
| | clusters across all donors | ≥1 cluster |
| Ab1 | 1 | 3/10 |
| Ab2 | 2 | 6/10 |
| Ab3 | 2 | 5/10 |
| Ab4 | 3 | 5/10 |
| Ab5 | 3 | 8/10 |

T cell proliferation assay: The ability of the exemplified anti-human TNFα antibodies MAPPs-derived peptide clusters to activate CD4+T cells by inducing cellular proliferation was assessed. CD8+T cells were depleted from cryopreserved PBMC's from 10 healthy donors and labeled with 1 μM Carboxyfluorescein Diacetate Succinimidyl Ester (CFSE). CD8+T cell depleted PBMCs were seeded at 4×10⁶ cells/mL/well in AIM-V media (Life Technologies, cat# 12055-083) with 5% CTS™ Immune Cell SR (Gibco, cat# A2596101) and tested in triplicate in 2.0 mL containing the different test molecules: DMSO control, media control, keyhole limpet haemocyanin (KLH; positive control), PADRE-X peptide (synthetic vaccine helper peptide, positive peptide control), or the respective anti-human TNFα antibody MAPPs-derived peptide clusters (10 μM each peptide). Cells were cultured and incubated for 7 days at 37° C. with 5% CO₂. On day 7, samples were stained with the following cell surface markers: anti-CD3, anti-CD4, anti-CD14, anti-CD19, and DAPI for viability detection by flow cytometry using a BD LSRFortessa™, equipped with a High Throughput Sampler (HTS). Data was analyzed using FlowJo® Software (FlowJo, LLC, TreeStar) and a Cellular Division Index (CDI) was calculated. Briefly, the CDI for each MAPPs-derived peptide cluster was calculated by dividing the percent of proliferating CFSE^{dim}CD4+T cells from peptide-stimulated wells by the percent of proliferating CFSE^{dim}CD4+T cells in the unstimulated wells. A CDI of≥2.5 was considered to represent a positive response. A percent donor frequency across all donors was evaluated.

The results as in Tables 13a and 13b, show that the LCDR1 (Table 13a) and HCDR3 (Table 13b) peptides for Ab2 induced a T cell response frequency in about 22.0% and 25% donors respectively, indicating a significantly reduced immunogenicity risk for Ab2 when compared to the positive controls. The KLH positive control induced a T cell response in 100% of donors, and the PADRE-X (Synthetic vaccine helper peptide) positive control, induced a T cell response in 67% and 62.5% of donors respectively in the two studies. This range fell within the expected range for this assay (48.1%+24.4 Positive Donor Frequency).

TABLE 13a
Frequency of CD4+ T cell responses induced by MAPPs-derived
peptides in healthy donors.

| | | Median | Median | | | Number of |
|---|---|---|---|---|---|---|
| | % | CDI | CDI | | | positive |
| Molecule | Positive | (Positive | (All | Range | | donors |
| Tested | Donors | Donors) | donors) | High | Low | (CDI > 2.5) |
| KLH | 100.0 | 190.8 | 190.8 | 1170.1 | 8.8 | 9/9 |
| PADRE-X | 67.0 | 4.0 | 3.1 | 17.8 | 0.5 | 6/9 |
| Ab2 | 22.0 | 5.5 | 1.1 | 6.0 | 0.6 | 2/9 |
| LCDR1 | | | | | | |
| peptide | | | | | | |

TABLE 13b
Frequency of CD4+ T cell responses induced by MAPPs-derived peptides
in healthy donors.

| | | Median | Median | | | |
|---|---|---|---|---|---|---|
| | % | CDI | CDI | | | Number |
| Molecule | Positive | (Positive | (All | Range | | of |
| Tested | Donors | Donors) | donors) | High | Low | donors |
| KLH | 100.0 | 230.2 | 230.2 | 3558.5 | 12.0 | 8/8 |
| PADRE-X | 62.5 | 15.3 | 7.5 | 45.5 | 0.3 | 5/8 |
| Ab2 HCDR3 | 25.0 | 7.2 | 1.3 | 7.7 | 0.1 | 2/8 |
| peptide | | | | | | |

### Example 7. Biophysical Properties of Exemplified Anti-Human TNFα Ab1 GC Conjugate of Example 1b

Biophysical properties of the exemplified anti-human TNFα Ab1 GC conjugate of Example 1b was evaluated for developability.

Example 7a. Viscosity: Samples of the anti-human TNFα Ab1 GC conjugate of Example 1b were concentrated to about 50 mg/mL, 100 mg/mL and 150 mg/mL in a common formulation buffer matrix at pH 6. The viscosity for the conjugate at all 3 concentrations was measured using a VROC® initium (RheoSense) at 15° C. using the average of 9 replicate measurements. The results in Table 15, showed that anti-human TNFα Ab1 GC conjugate of Example 1b at 50 mg/mL (2 cP), 100 mg/mL (4.7 cP) and 125 mg/mL (8.2 cP) had good viscosity profiles. The viscosity for the exemplified anti-human TNFα Ab1 GC conjugate at 125 mg/mL (8.2 cP) was similar to that of the unconjugated anti-human TNFα Ab1 at 125 mg/mL (8.8 cP), indicating that conjugating the linker-payload to the 4 engineered cysteines on the exemplified anti-human TNFα Ab1 surface did not negatively impact viscosity.

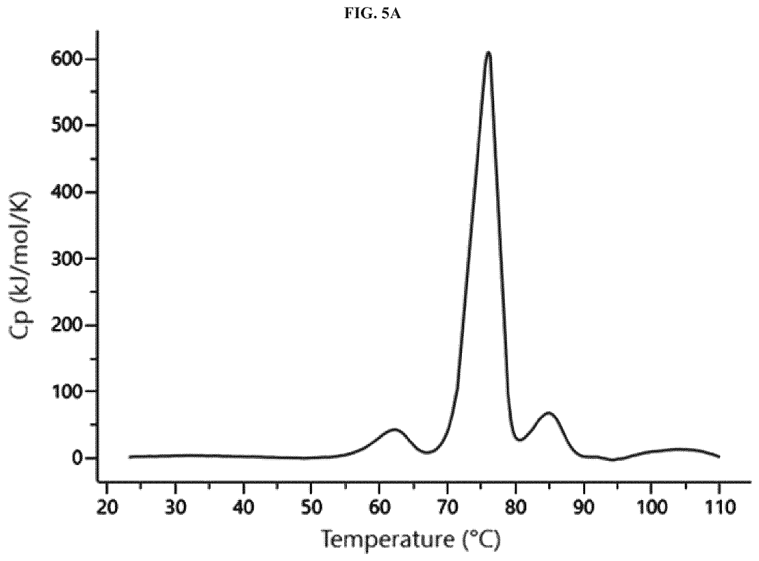

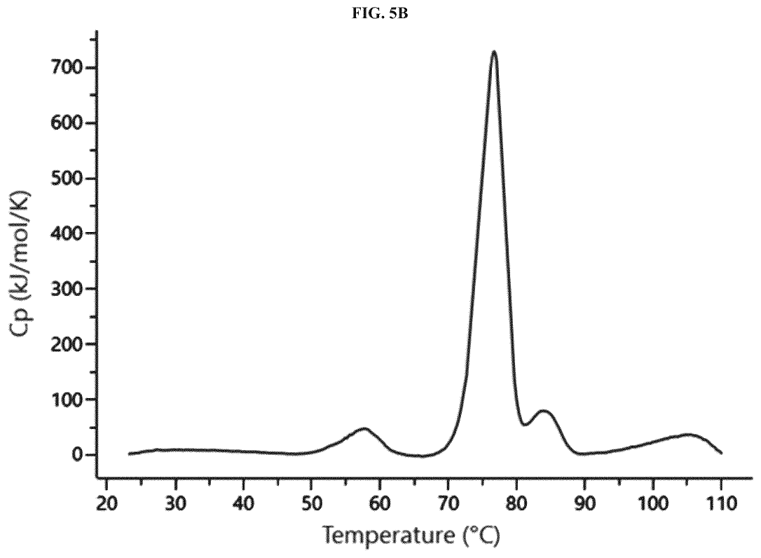

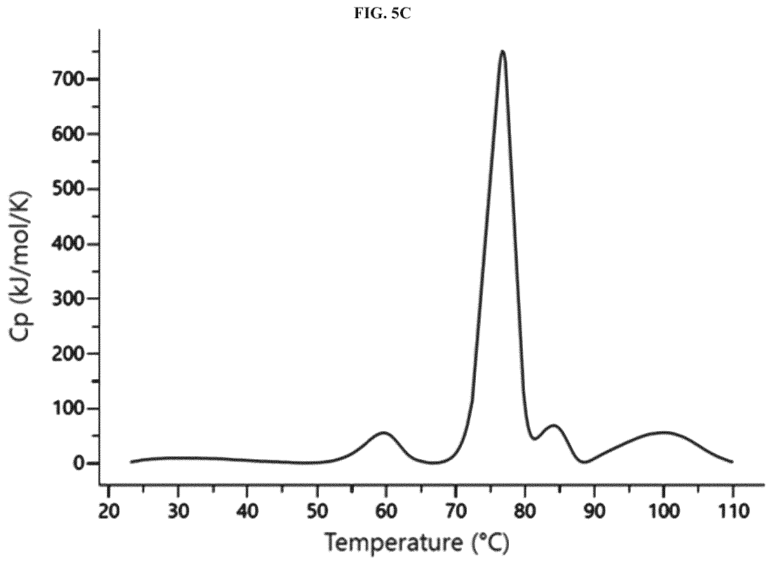

Example 7b. Thermal stability: Differential Scanning calorimetry (DSC) was used to evaluate the stability of the anti-human TNFα Ab1 GC conjugate of Example 1b against thermal denaturation. The onset of melting (Tonset) and thermal melting temperatures (TM1, TM2 and TM3) of the exemplified anti-human TNFα Ab1 GC conjugate in PBS, pH 7.2 buffer, Acetate, pH 5 and Histidine, pH 6 were obtained by data fitting and are listed in Table 14. Thermograms for the 3 buffer compositions are depicted in FIG. 5A, 5B, and 5C. Thermal transitions for each domain were well resolved and the results in

Table 14 show that the anti-human TNFα Ab1 GC conjugate of Example 1b has good thermal stability.

Example 7c. Aggregation upon temperature stress: The solution stability of the anti-human TNFα Ab1 GC conjugate of Example 1b over time was assessed at approximately 100 mg/mL and 50 mg/mL in a common 5 mM histidine pH 6.0 buffer with excipients. Samples were incubated for a period of 28 days at 5° C. and 35° C. Following incubation, samples were analyzed for the percentage of high molecular weight (% HMW) species using size exclusion chromatography (SEC-HPLC). The results in Table 15, show the anti-human TNFα Ab1 GC conjugate of Example 1b has an acceptable aggregation profile over a 4-week period at either 5° C. or 35° C.

Example 7d. Pharmacokinetics: PK profile of the anti-human TNFα Ab1 GC conjugate of Example 1b in cynomolgus monkeys was found to have an acceptable developability profile.

TABLE 14
Thermal stability (º C.) of exemplified anti-human
TNFα Ab1 GC conjugate of Example 1b

| | Buffer | Tonset | TM1 | TM2 | TM3 |
|---|---|---|---|---|---|
| Ab1 GC conjugate | PBS, pH 7.2 | 57.3 | 61.9 | 75.5 | 84.7 |
| | Acetate pH 5 | 52.7 | 57.0 | 75.4 | 84.0 |
| | Histidine pH 6 | 54.7 | 59.1 | 75.5 | 84.1 |

TABLE 15
Viscosity and high concentration temperature hold stability for the
exemplified anti-human TNFα Ab1 GC conjugate of Example 1b

| | Ab1 GC conjugate | |
|---|---|---|
| Concentration | 50 mg/mL | 100 mg/mL |
| Viscosity (cP) | 2 | 4.7 |
| % HMW after 4-week | 0.04 | 0.2 |
| incubation at 5° C. | | |
| % HMW change after | 1.4 | 2.9 |
| 4-week incubation at | | |
| 35° C. | | |

### Example 8. In vivo Function of the Anti-Human TNFα Ab GC Conjugates of Example 1b, Example 1c, and Example 1d

Example 8a. in vivo inhibition of human TNFα-induced CXCL1 cytokine: Neutralization of TNFα-induced CXCL1 by the exemplified anti-human TNFα Ab1 GC conjugate of Example 1b and anti-human TNFα Ab1 was assessed in vivo. Administration of human TNFα to C57/B6 mice induces a rapid and transient increase of mouse plasma CXCL1 levels. This allows for the interrogation of the neutralization capacity of the exemplified anti-human TNFα Ab1 and the anti-human TNFα Ab1 GC conjugate in vivo.

Briefly, C57/B6 mice (N=8/group) were dosed 0.3 mg/kg or 3 mg/kg subcutaneously (SC) with the exemplified anti-human TNFα Ab1 GC conjugate or anti-human TNFα Ab1 and 3 mg/kg of a non-binding isotype control. Twenty-four hours post dosing, the mice were challenged with human TNFα via intraperitoneal injection at a dose of 3 μg/mouse. Two hours post human TNFα challenge the mice were sacrificed, blood was collected, and clarified to plasma by centrifugation. Plasma was analyzed for mouse CXCL1 concentration using a commercial MSD assay (MesoScale Discovery, P/N. K152QTG-1) according to manufacturer's instructions.

The results in Table 16, show that the exemplified anti-human TNFα Ab1 and anti-human TNFα Ab1 GC conjugate of Example 1b significantly inhibited in vivo human TNFα-induced plasma CXCL1 production, relative to isotype control treated mice (p<0.001, ANOVA followed by Tukey's Multiple Comparison test) by about 80.5 and 81.6% at 3 mg/kg, and by about 69.4 and 58.9% at 0.3 mg/kg, respectively. Importantly, there were no significant difference between the anti-human TNFα Ab1 GC conjugate and the anti-human TNFα Ab1 at the doses tested. This indicates that the exemplified anti-human TNFα Ab1 neutralizes the biological effects induced by human TNFα in vivo and conjugation to the GC does not impact this function.

TABLE 16
in vivo inhibition of human TNFα-induced CXCL1 cytokine production
by the anti-human TNFa Ab1 GC conjugate of Example 1b

| | | Plasma CXCL1 concentration | | | |
|---|---|---|---|---|---|
| | Dose | Mean | ± SEM | % | P Value |
| | mg/kg | (pg/mL) | (pg/mL) | Inhibition | vs Isotype |
| IgG1 isotype | 3 | 1058.70 | 170.84 | 0.0% | |
| control | | | | | |
| Ab1 | 3 | 236.69 | 48.31 | 80.5% | <0.0001 |
| Ab1 | 0.3 | 350.57 | 82.52 | 69.4% | 0.0001 |
| Ab1 GC conjugate | 3 | 226.14 | 37.48 | 81.6% | <0.0001 |
| Ab1 GC conjugate | 0.3 | 457.12 | 89.21 | 58.9% | 0.0013 |

Ordinary one-way ANOVA, Tukey test, multiple comparison

Example 8b. In vivo efficacy in a type IV hypersensitivity of a fully humanized mouse model: A humanized mouse model of contact hypersensitivity was used to determine in vivo activity of the anti-human TNFα Ab1 GC conjugate of Example 1b and Example 1c and anti-human TNFα Ab1.

Immunodeficient NOG mice expressing human GM-CSF and human IL-3 to support myeloid lineage development (huNOG-EXL, Taconic) were engrafted at 6 weeks of age with human CD34+hematopoietic stem cells isolated from human cord blood. 20-24 weeks after stem cell administration the mice were assessed for sufficient human CD45 engraftment (>25% in blood) and subjected to an oxazolone-induced contact hypersensitivity protocol. On day 0, mice grouped by body weight were dosed at 1 mg/kg subcutaneously (SC) with either anti-human TNFα Ab1 GC conjugate of Example 1b (n=7), TNFα Ab1 GC conjugate of Example 1c (n=7), anti-human TNFα Ab1 (n=7), an exemplary anti-human TNFα Ab GC conjugate from US2020338208 (n=7), or a control human IgG1 antibody (n=7). On day 1, mice were anesthetized with 5% isoflurane, their abdomens shaved, and 100 μL of 3% oxazolone in ethanol was applied to the shaved area. Mice were dosed again on day 7 at 1 mg/kg SC, anesthetized, and then challenged with 2% oxazolone in ethanol on both ears (10 μL/side/ear) 24 hours post dose. The dose challenge paradigm was repeated weekly; with the dose of test agent increased to 3 mg/kg for Challenge 2 and to 10 mg/kg for Challenge 3. The inflammatory response was determined by the difference in ear thickness prior to and 24 hours following each challenge using a Miltenyi Biotec electronic caliper. P-values between groups were calculated by one-way ANOVA followed by Tukey's post hoc test and considered significant if<0.05 (GraphPad Prism).

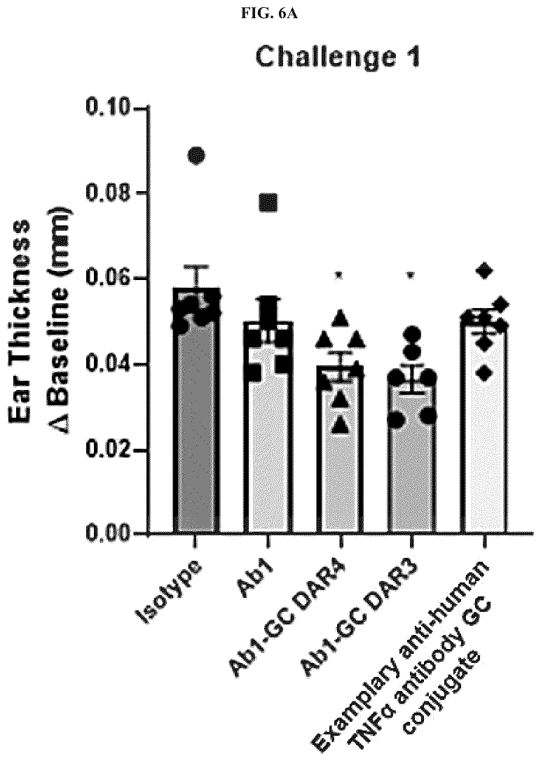

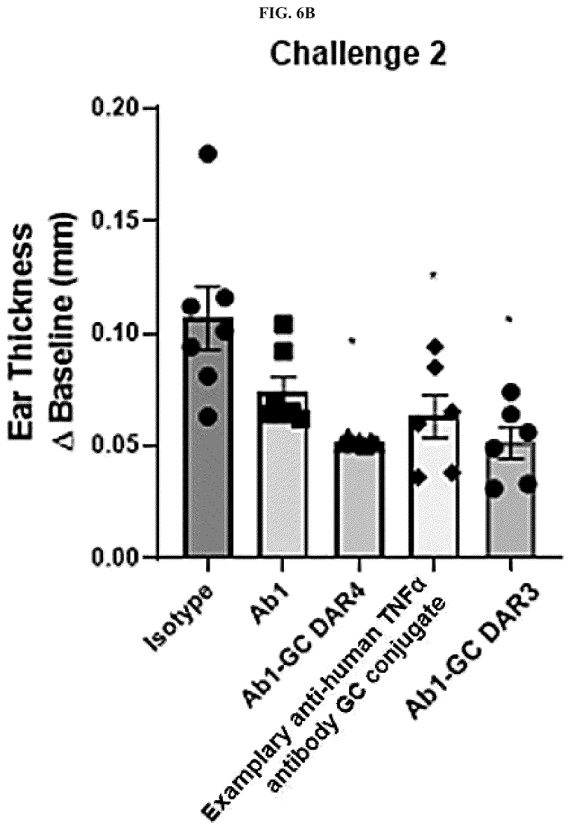

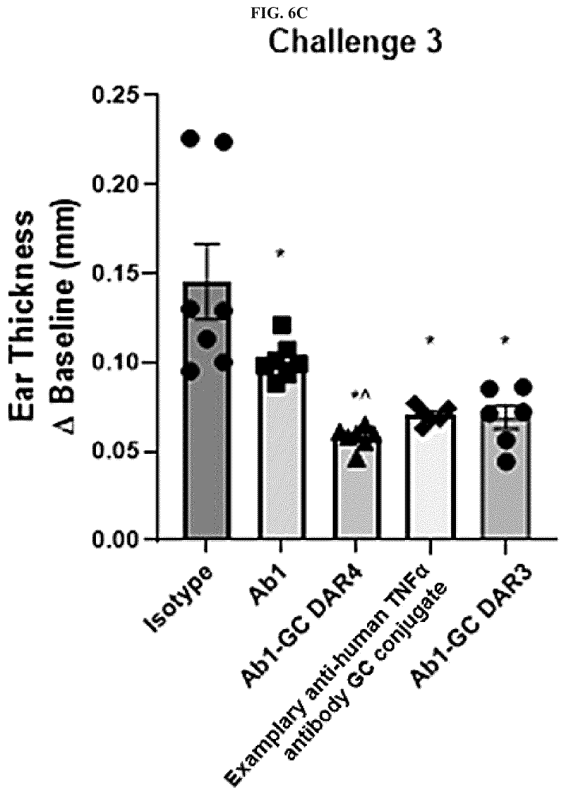

The results in Table 17 and FIGS. 6A-6C, show that the anti-human TNFα Ab1 GC conjugate of Example 1b and Example 1c elicited superior reduction in the in vivo inflammatory responses from hapten-induced contact hypersensitivity reaction at all 3 challenges (1, 3, and 10 mg/kg) when compared to both the anti-human TNFα Ab1 which attenuated the ear swelling only at the 10 mg/kg challenge 3 dose, and to the exemplary anti-human TNFα Ab GC conjugate from US2020338208. Furthermore, the results show that conjugation of the anti-human TNFα Ab1 to 3 or 4 GC molecules (DAR) elicited similar efficacy. These results show that the anti-human TNFα Ab1 GC conjugate effectively delivered the glucocorticoid to the inflamed tissue and that the glucocorticoid significantly abrogated the biological effects associated with a type IV hypersensitivity reaction in a humanized mouse model, indicating that this anti-inflammatory response could be elicited in a human subject.

TABLE 17
In vivo efficacy of the anti-human TNFα Ab1 GC conjugates of Example
1b and Example 1c in a type IV hypersensitivity humanized mouse model

| | Challenge 1 | | Challenge 2 | | Challenge 3 | |
|---|---|---|---|---|---|---|
| | 1 mg/kg | | 3 mg/kg | | 10 mg/kg | |
| | Δ Ear thickness | | Δ Ear thickness | | Δ Ear thickness | |
| | (mm) | | (mm) | | (mm) | |
| | Mean | SEM | Mean | SEM | Mean | SEM |
| hIgG1 Isotype | 0.058 | 0.005 | 0.107 | 0.014 | 0.145 | 0.021 |
| Control | | | | | | |
| Ab1 | 0.050 | 0.005 | 0.074 | 0.006 | 0.101* | 0.004 |
| Ab1 GC | 0.039* | 0.003 | 0.052* | 0.001 | 0.058*\hat{ } | 0.002 |
| conjugate DAR | | | | | | |
| 4 | | | | | | |
| Ab1 GC | 0.036* | 0.003 | 0.051* | 0.007 | 0.069* | 0.007 |
| conjugate DAR | | | | | | |
| 3 | | | | | | |
| exemplary anti- | 0.050 | 0.003 | 0.063* | 0.010 | 0.07* | 0.002 |
| human TNFα | | | | | | |
| GC conjugate | | | | | | |
| from | | | | | | |
| US2020338208 | | | | | | |

*p < 0.05 vs Isotype; ANOVA Tukey;
\hat{ }p < 0.05 vs Ab1; ANOVA Tukey

Example 8c. In vivo efficacy in human TNFα transgenic mouse polyarthritis model: A human TNFα transgenic mouse polyarthritis model (Taconic, # 1006) was used to evaluate the efficacy of the the anti-human TNFα Ab2 GC conjugate of Example 1d and anti-human TNFα Ab2, as primary treatment in adalimumab naive mice and as secondary treatment in adalimumab treated mice which developed anti-drug antibodies to adalimumab, and have a diminished or loss or response to adalimumab, i.e., adalimumab refractory mice. This mouse model constitutively expresses human TNFα via a CMV-promoter which results in progressive joint inflammation manifesting primarily in the fore and hind paws. Treatment with adalimumab attenuates the disease progression for a few weeks; however, the beneficial effects wane due to the development of neutralizing anti-drug antibodies. In order to obviate the potential for adalimumab to generate anti-drug antibodies to the human Fc portion of the antibody and thus affect the activity of the anti-human TNFα Ab2 GC conjugate of Example 1d and anti-human TNFα Ab2, all molecules were generated as chimeric species, wherein the antibody constant domains were replaced with those of a mouse IgG2a antibody.

At 13 weeks of age, once all mice demonstrated moderate inflammation in one or more paws (score 4-9), mice were divided into 6 clinical score matched groups of 8 mice/group. Mice in each group were dosed weekly at 3 mg/kg subcutaneously (SC) for 9 weeks with either mIgG2a isotype control, human/mouse chimeric anti-human TNFα Ab2 (herein referred to as “h/mAb2”), human/mouse chimeric anti-human TNFα Ab2 GC conjugate (herein referred to as “h/mAb2-GC”), human/mouse chimeric adalimumab (herein referred to as “h/m-adalimumab”), h/m-adalimumab for 2 doses then switched to human/mouse chimeric adalimumab GC conjugate (herein referred to as “h/m-adalimumab-GC”; prepared with engineered cysteines and conjugated to GC-L essentially as described in Example 1b) for the duration of the study, and h/m-adalimumab for 2 doses then switched to h/mAb2-GC for the duration of the study. The parameters for clinical scores for each limb were as follows: 0=no evidence of distortion; 1=mild distortion; 2=moderate distortion; 3=severe distortion/mild swelling; 4=severe distortion/severe swelling/loss of function. Mice were scored twice weekly and weighed routinely. Blood was collected on Day 10 to quantitate antibody exposure levels and to determine the degree of anti-drug antibody development. Upon termination of the experiment, mice were anesthetized with isoflurane and blood and tissues were harvested.

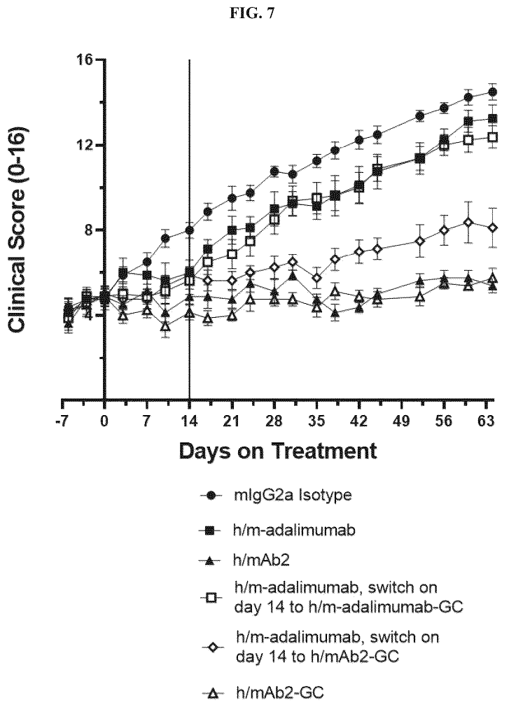

The results in FIG. 7, show that h/mAb2-GC and h/mAb2 completely arrested the progression of disease upon initiation of treatment and lasted for the duration of the 9-week treatment, as measured by clinical score compared to all other treatments. Importantly, this indicated that h/mAb2-GC and h/mAb2 did not generate a significant anti-drug antibody response, such that it would neutralize and/or diminish efficacy of the conjugate or the antibody. The results also showed that h/m-adalimumab was able to delay the progression of disease for about 2 weeks; however, efficacy was lost coincident with the appearance of anti-drug antibodies to h/m-adalimumab by about week 2 of the 9-week treatment. However, importantly, mice treated with h/m-adalimumab for 2 weeks and then switched to treatment with h/mAb2-GC maintained a significant abrogation of disease progression, whereas mice treated with h/m-adalimumab for 2 weeks and then switched to treatment with h/m-adalimumab-GC displayed an inflammatory response that mirrored the h/m-adalimumab treatment alone group. These results indicate that the anti-human TNFα Ab2 GC conjugate has low to no cross-reactivity to anti-drug antibodies against adalimumab. These results indicate the potential use of the exemplified anti-human TNFα Ab conjugates in the treatment of subjects who develop anti-drug antibodies against other anti-TNFα therapeutics such as adalimumab and have diminished response to that treatment.

SEQUENCE LISTING
Ab1
HCDR1 for Ab1, Ab2, and Ab6
SEQ ID NO: 1
GYTFTGYYIH
HCDR2 for Ab1, Ab2, and Ab6
SEQ ID NO: 2
WINPYTGGTNYAQKFQG
HCDR3 for Ab1
SEQ ID NO: 3
DLYGSSNYGGDV
LCDR1 for Ab1 and Ab3
SEQ ID NO: 4
QASQGISNYLN
LCDR2 for Ab1, Ab2, Ab3, Ab4, Ab5, and Ab6
SEQ ID NO: 5
DASNLET
LCDR3 for Ab1, Ab3, and Ab5
SEQ ID NO: 6
QQYDKLPLT
VH for Ab1
SEQ ID NO: 7
QVQLVQSGAEVKKPGASVKVSCKASGYTFTGYYIHWVRQAPGQGLEWMGW
INPYTGGTNYAQKFQGRVTMTRDTSISTAYMELSRLRSDDTAVYYCARDL
YGSSNYGGDVWGQGTTVTVSS
VL for Ab1 and Ab3
SEQ ID NO: 8
DIQMTQSPSSLSASVGDRVTITCQASQGISNYLNWYQQKPGKAPKLLIYD
ASNLETGVPSRFSGSGSGTDFTFTISSLQPEDIATYYCQQYDKLPLTFGG
GTKVEIK
HC for Ab1
SEQ ID NO: 9
QVQLVQSGAEVKKPGASVKVSCKASGYTFTGYYIHWVRQAPGQGLEWMGW
INPYTGGTNYAQKFQGRVTMTRDTSISTAYMELSRLRSDDTAVYYCARDL
YGSSNYGGDVWGQGTTVTVSSASTKGPCVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDICVEWESNGQPENNYKTTPP
VLDSDGSFFLYSKLTVDKSRWQQGNVFSCSVMHEALHNHYTQKSLSLSPG
K
LC for Ab1 and Ab3
SEQ ID NO: 10
DIQMTQSPSSLSASVGDRVTITCQASQGISNYLNWYQQKPGKAPKLLIYD
ASNLETGVPSRFSGSGSGTDFTFTISSLQPEDIATYYCQQYDKLPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
HC DNA for Ab1
SEQ ID NO: 11
CAGGTGCAGCTGGTGCAGTCTGGGGCTGAGGTGAAGAAGCCTGGGGCCTC
AGTGAAGGTCTCCTGCAAGGCTTCTGGATACACCTTCACCGGCTACTATA
TACACTGGGTGCGACAGGCCCCTGGACAAGGGCTTGAGTGGATGGGATGG
ATCAACCCTTACACCGGTGGCACAAACTATGCACAGAAGTTTCAGGGCAG
GGTCACCATGACCAGGGACACGTCCATCAGCACAGCCTACATGGAGCTGA
GCAGGCTGAGATCTGACGACACGGCCGTGTATTACTGTGCGAGAGATCTC
TATGGTTCGAGTAATTACGGTGGCGACGTCTGGGGCCAAGGGACCACGGT
CACCGTCTCCTCAGCTAGCACCAAGGGCCCATGTGTCTTCCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAGAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTATGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAAGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGAGGAGATGACCAAGAACCAAGT
CAGCCTGACCTGCCTGGTCAAAGGCTTCTATCCCAGCGACATCTGCGTGG
AGTGGGAGAGCAATGGGCAGCCGGAGAACAACTACAAGACCACGCCTCCC
GTGCTGGACTCCGACGGCTCCTTCTTCCTCTATTCCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGC
AAA
LC DNA for Ab1 and Ab3
SEQ ID NO: 12
GACATCCAGATGACCCAGTCTCCATCCTCCCTGTCTGCATCTGTAGGAGA
CAGAGTCACCATCACTTGCCAGGCGAGTCAGGGCATTAGCAACTATTTAA
ATTGGTATCAGCAGAAACCAGGGAAAGCCCCTAAGCTCCTGATCTACGAT
GCATCCAATTTGGAAACAGGGGTCCCATCAAGGTTCAGTGGAAGTGGATC
TGGGACAGATTTTACTTTCACCATCAGCAGCCTGCAGCCTGAAGATATTG
CAACATATTACTGTCAACAGTATGATAAGCTCCCGCTCACTTTCGGCGGA
GGGACCAAGGTGGAGATCAAACGGACCGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGC
Ab2
HCDR1 for Ab1, Ab2, and Ab6
SEQ ID NO: 1
GYTFTGYYIH
HCDR2 for Ab1, Ab2, and Ab6
SEQ ID NO: 2
WINPYTGGTNYAQKFQG
HCDR3 for Ab2, Ab3, Ab4, and Ab5
SEQ ID NO: 13
DLYGSSNYGMDV
LCDR1 for Ab2, Ab4, and Ab5
SEQ ID NO: 14
QASQGIRNYLN
LCDR2 for Ab1, Ab2, Ab3, Ab4, Ab5, and Ab6
SEQ ID NO: 5
DASNLET
LCDR3 for Ab2 and Ab4
SEQ ID NO: 15
QQYDNLPLT
VH for Ab2
SEQ ID NO: 16
QVQLVQSGAEVKKPGASVKVSCKASGYTFTGYYIHWVRQAPGQGLEWMGW
INPYTGGTNYAQKFQGRVTMTRDTSISTAYMELSRLRSDDTAVYYCARDL
YGSSNYGMDVWGQGTTVTVSS
VL for Ab2 and Ab4
SEQ ID NO: 17
DIQMTQSPSSLSASVGDRVTITCQASQGIRNYLNWYQQKPGKAPKLLIYD
ASNLETGVPSRFSGSGSGTDFTFTISSLQPEDIATYYCQQYDNLPLTFGG
GTKVEIK
HC for Ab2
SEQ ID NO: 18
QVQLVQSGAEVKKPGASVKVSCKASGYTFTGYYIHWVRQAPGQGLEWMGW
INPYTGGTNYAQKFQGRVTMTRDTSISTAYMELSRLRSDDTAVYYCARDL
YGSSNYGMDVWGQGTTVTVSSASTKGPCVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDICVEWESNGQPENNYKTTPP
VLDSDGSFFLYSKLTVDKSRWQQGNVFSCSVMHEALHNHYTQKSLSLSPG
K
LC for Ab2 and Ab4
SEQ ID NO: 19
DIQMTQSPSSLSASVGDRVTITCQASQGIRNYLNWYQQKPGKAPKLLIYD
ASNLETGVPSRFSGSGSGTDFTFTISSLQPEDIATYYCQQYDNLPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
HC DNA for Ab2
SEQ ID NO: 20
CAGGTGCAGCTGGTGCAGTCTGGGGCTGAGGTGAAGAAGCCTGGGGCCTC
AGTGAAGGTCTCCTGCAAGGCTTCTGGATACACCTTCACCGGCTACTATA
TACACTGGGTGCGACAGGCCCCTGGACAAGGGCTTGAGTGGATGGGATGG
ATCAACCCTTACACCGGTGGCACAAACTATGCACAGAAGTTTCAGGGCAG
GGTCACCATGACCAGGGACACGTCCATCAGCACAGCCTACATGGAGCTGA
GCAGGCTGAGATCTGACGACACGGCCGTGTATTACTGTGCGAGAGATCTC
TATGGTTCGAGTAATTACGGTATGGACGTCTGGGGCCAAGGGACCACGGT
CACCGTCTCCTCAGCTAGCACCAAGGGCCCATGCGTCTTCCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAGAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTATGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAAGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGAGGAGATGACCAAGAACCAAGT
CAGCCTGACCTGCCTGGTCAAAGGCTTCTATCCCAGCGACATCTGCGTGG
AGTGGGAGAGCAATGGGCAGCCGGAGAACAACTACAAGACCACGCCTCCC
GTGCTGGACTCCGACGGCTCCTTCTTCCTCTATTCCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGC
AAA
LC DNA for Ab2 and Ab4
SEQ ID NO: 21
GACATCCAGATGACCCAGTCTCCATCCTCCCTGTCTGCATCTGTAGGAGA
CAGAGTCACCATCACTTGCCAGGCGAGTCAGGGCATTCGCAACTATTTAA
ATTGGTATCAGCAGAAACCAGGGAAAGCCCCTAAGCTCCTGATCTACGAT
GCATCCAATTTGGAAACAGGGGTCCCATCAAGGTTCAGTGGAAGTGGATC
TGGGACAGATTTTACTTTCACCATCAGCAGCCTGCAGCCTGAAGATATTG
CAACATATTACTGTCAACAGTATGATAACCTCCCGCTCACTTTCGGCGGA
GGGACCAAGGTGGAGATCAAACGGACCGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGC
Ab3
HCDR1 for Ab3, Ab4, and Ab5
SEQ ID NO: 22
GYTFTGYYMH
HCDR2 for Ab3, Ab4, and Ab5
SEQ ID NO: 23
WINPYTGGTKYAQKFQG
HCDR3 for Ab2, Ab3, Ab4, and Ab5
SEQ ID NO: 13
DLYGSSNYGMDV
LCDR1 for Ab1 and Ab3
SEQ ID NO: 4
QASQGISNYLN
LCDR2 for Ab1, Ab2, Ab3, Ab4, Ab5, and Ab6
SEQ ID NO: 5
DASNLET
LCDR3 for Ab1, Ab3, and Ab5
SEQ ID NO: 6
QQYDKLPLT
VH for Ab3, Ab4, and Ab5
SEQ ID NO: 24
QVQLVQSGAEVKKPGASVKVSCKASGYTFTGYYMHWVRQAPGQGLEWMGW
INPYTGGTKYAQKFQGRVTMTRDTSISTAYMELSRLRSDDTAVYYCARDL
YGSSNYGMDVWGQGTTVTVSS
VL for Ab1 and Ab3
SEQ ID NO: 8
DIQMTQSPSSLSASVGDRVTITCQASQGISNYLNWYQQKPGKAPKLLIYD
ASNLETGVPSRFSGSGSGTDFTFTISSLQPEDIATYYCQQYDKLPLTFGG
GTKVEIK
HC for Ab3, Ab4, and Ab5
SEQ ID NO: 25
QVQLVQSGAEVKKPGASVKVSCKASGYTFTGYYMHWVRQAPGQGLEWMGW
INPYTGGTKYAQKFQGRVTMTRDTSISTAYMELSRLRSDDTAVYYCARDL
YGSSNYGMDVWGQGTTVTVSSASTKGPCVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDICVEWESNGQPENNYKTTPP
VLDSDGSFFLYSKLTVDKSRWQQGNVFSCSVMHEALHNHYTQKSLSLSPG
K
LC for Ab1 and Ab3
SEQ ID NO: 10
DIQMTQSPSSLSASVGDRVTITCQASQGISNYLNWYQQKPGKAPKLLIYD
ASNLETGVPSRFSGSGSGTDFTFTISSLQPEDIATYYCQQYDKLPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
HC DNA for Ab3, Ab4, and Ab5
SEQ ID NO: 26
CAGGTGCAGCTGGTGCAGTCTGGGGCTGAGGTGAAGAAGCCTGGGGCCTC
AGTGAAGGTCTCCTGCAAGGCTTCTGGATACACCTTCACCGGCTACTATA
TGCACTGGGTGCGACAGGCCCCTGGACAAGGGCTTGAGTGGATGGGATGG
ATCAACCCTTACACCGGTGGCACAAAGTATGCACAGAAGTTTCAGGGCAG
GGTCACCATGACCAGGGACACGTCCATCAGCACAGCCTACATGGAGCTGA
GCAGGCTGAGATCTGACGACACGGCCGTGTATTACTGTGCGAGAGATCTC
TATGGTTCGAGTAATTACGGTATGGACGTCTGGGGCCAAGGGACCACGGT
CACCGTCTCCTCAGCTAGCACCAAGGGCCCATGCGTCTTCCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAGAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTATGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAAGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGAGGAGATGACCAAGAACCAAGT
CAGCCTGACCTGCCTGGTCAAAGGCTTCTATCCCAGCGACATCTGCGTGG
AGTGGGAGAGCAATGGGCAGCCGGAGAACAACTACAAGACCACGCCTCCC
GTGCTGGACTCCGACGGCTCCTTCTTCCTCTATTCCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGC
AAA
LC DNA for Ab1 and Ab3
SEQ ID NO: 12
GACATCCAGATGACCCAGTCTCCATCCTCCCTGTCTGCATCTGTAGGAGA
CAGAGTCACCATCACTTGCCAGGCGAGTCAGGGCATTAGCAACTATTTAA
ATTGGTATCAGCAGAAACCAGGGAAAGCCCCTAAGCTCCTGATCTACGAT
GCATCCAATTTGGAAACAGGGGTCCCATCAAGGTTCAGTGGAAGTGGATC
TGGGACAGATTTTACTTTCACCATCAGCAGCCTGCAGCCTGAAGATATTG
CAACATATTACTGTCAACAGTATGATAAGCTCCCGCTCACTTTCGGCGGA
GGGACCAAGGTGGAGATCAAACGGACCGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGC
Ab4
HCDR1 for Ab3, Ab4, and Ab5
SEQ ID NO: 22
GYTFTGYYMH
HCDR2 for Ab3, Ab4, and Ab5
SEQ ID NO: 23
WINPYTGGTKYAQKFQG
HCDR3 for Ab2, Ab3, Ab4, and Ab5
SEQ ID NO: 13
DLYGSSNYGMDV
LCDR1 for Ab2, Ab4, and Ab5
SEQ ID NO: 14
QASQGIRNYLN
LCDR2 for Ab1, Ab2, Ab3, Ab4, Ab5, and Ab6
SEQ ID NO: 5
DASNLET
LCDR3 for Ab2 and Ab4
SEQ ID NO: 15
QQYDNLPLT
VH for Ab3, Ab4, and Ab5
SEQ ID NO: 24
QVQLVQSGAEVKKPGASVKVSCKASGYTFTGYYMHWVRQAPGQGLEWMGW
INPYTGGTKYAQKFQGRVTMTRDTSISTAYMELSRLRSDDTAVYYCARDL
YGSSNYGMDVWGQGTTVTVSS
VL for Ab2 and Ab4
SEQ ID NO: 17
DIQMTQSPSSLSASVGDRVTITCQASQGIRNYLNWYQQKPGKAPKLLIYD
ASNLETGVPSRFSGSGSGTDFTFTISSLQPEDIATYYCQQYDNLPLTFGG
GTKVEIK
HC for Ab3, Ab4, and Ab5
SEQ ID NO: 25
QVQLVQSGAEVKKPGASVKVSCKASGYTFTGYYMHWVRQAPGQGLEWMGW
INPYTGGTKYAQKFQGRVTMTRDTSISTAYMELSRLRSDDTAVYYCARDL
YGSSNYGMDVWGQGTTVTVSSASTKGPCVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDICVEWESNGQPENNYKTTPP
VLDSDGSFFLYSKLTVDKSRWQQGNVFSCSVMHEALHNHYTQKSLSLSPG
K
LC for Ab2 and Ab4
SEQ ID NO: 19
DIQMTQSPSSLSASVGDRVTITCQASQGIRNYLNWYQQKPGKAPKLLIYD
ASNLETGVPSRFSGSGSGTDFTFTISSLQPEDIATYYCQQYDNLPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
HC DNA for Ab3, Ab4, and Ab5
SEQ ID NO: 26
CAGGTGCAGCTGGTGCAGTCTGGGGCTGAGGTGAAGAAGCCTGGGGCCTC
AGTGAAGGTCTCCTGCAAGGCTTCTGGATACACCTTCACCGGCTACTATA
TGCACTGGGTGCGACAGGCCCCTGGACAAGGGCTTGAGTGGATGGGATGG
ATCAACCCTTACACCGGTGGCACAAAGTATGCACAGAAGTTTCAGGGCAG
GGTCACCATGACCAGGGACACGTCCATCAGCACAGCCTACATGGAGCTGA
GCAGGCTGAGATCTGACGACACGGCCGTGTATTACTGTGCGAGAGATCTC
TATGGTTCGAGTAATTACGGTATGGACGTCTGGGGCCAAGGGACCACGGT
CACCGTCTCCTCAGCTAGCACCAAGGGCCCATGCGTCTTCCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAGAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTATGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAAGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGAGGAGATGACCAAGAACCAAGT
CAGCCTGACCTGCCTGGTCAAAGGCTTCTATCCCAGCGACATCTGCGTGG
AGTGGGAGAGCAATGGGCAGCCGGAGAACAACTACAAGACCACGCCTCCC
GTGCTGGACTCCGACGGCTCCTTCTTCCTCTATTCCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGC
AAA
LC DNA for Ab2 and Ab4
SEQ ID NO: 21
GACATCCAGATGACCCAGTCTCCATCCTCCCTGTCTGCATCTGTAGGAGA
CAGAGTCACCATCACTTGCCAGGCGAGTCAGGGCATTCGCAACTATTTAA
ATTGGTATCAGCAGAAACCAGGGAAAGCCCCTAAGCTCCTGATCTACGAT
GCATCCAATTTGGAAACAGGGGTCCCATCAAGGTTCAGTGGAAGTGGATC
TGGGACAGATTTTACTTTCACCATCAGCAGCCTGCAGCCTGAAGATATTG
CAACATATTACTGTCAACAGTATGATAACCTCCCGCTCACTTTCGGCGGA
GGGACCAAGGTGGAGATCAAACGGACCGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGC
Ab5
HCDR1 for Ab3, Ab4, and Ab5
SEQ ID NO: 22
GYTFTGYYMH
HCDR2 for Ab3, Ab4, and Ab5
SEQ ID NO: 23
WINPYTGGTKYAQKFQG
HCDR3 for Ab2, Ab3, Ab4, and Ab5
SEQ ID NO: 13
DLYGSSNYGMDV
LCDR1 for Ab2, Ab4, and Ab5
SEQ ID NO: 14
QASQGIRNYLN
LCDR2 for Ab1, Ab2, Ab3, Ab4, Ab5, and Ab6
SEQ ID NO: 5
DASNLET
LCDR3 for Ab1, Ab3, and Ab5
SEQ ID NO: 6
QQYDKLPLT
VH for Ab3, Ab4, and Ab5
SEQ ID NO: 24
QVQLVQSGAEVKKPGASVKVSCKASGYTFTGYYMHWVRQAPGQGLEWMGW
INPYTGGTKYAQKFQGRVTMTRDTSISTAYMELSRLRSDDTAVYYCARDL
YGSSNYGMDVWGQGTTVTVSS
VL for Ab5
SEQ ID NO: 27
DIQMTQSPSSLSASVGDRVTITCQASQGIRNYLNWYQQKPGKAPKLLIYD
ASNLETGVPSRFSGSGSGTDFTFTISSLQPEDIATYYCQQYDKLPLTFGG
GTKVEIK
HC for Ab3, Ab4, and Ab5
SEQ ID NO: 25
QVQLVQSGAEVKKPGASVKVSCKASGYTFTGYYMHWVRQAPGQGLEWMGW
INPYTGGTKYAQKFQGRVTMTRDTSISTAYMELSRLRSDDTAVYYCARDL
YGSSNYGMDVWGQGTTVTVSSASTKGPCVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDICVEWESNGQPENNYKTTPP
VLDSDGSFFLYSKLTVDKSRWQQGNVFSCSVMHEALHNHYTQKSLSLSPG
K
LC for Ab5
SEQ ID NO: 28
DIQMTQSPSSLSASVGDRVTITCQASQGIRNYLNWYQQKPGKAPKLLIYD
ASNLETGVPSRFSGSGSGTDFTFTISSLQPEDIATYYCQQYDKLPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
HC DNA for Ab3, Ab4, and Ab5
SEQ ID NO: 26
CAGGTGCAGCTGGTGCAGTCTGGGGCTGAGGTGAAGAAGCCTGGGGCCTC
AGTGAAGGTCTCCTGCAAGGCTTCTGGATACACCTTCACCGGCTACTATA
TGCACTGGGTGCGACAGGCCCCTGGACAAGGGCTTGAGTGGATGGGATGG
ATCAACCCTTACACCGGTGGCACAAAGTATGCACAGAAGTTTCAGGGCAG
GGTCACCATGACCAGGGACACGTCCATCAGCACAGCCTACATGGAGCTGA
GCAGGCTGAGATCTGACGACACGGCCGTGTATTACTGTGCGAGAGATCTC
TATGGTTCGAGTAATTACGGTATGGACGTCTGGGGCCAAGGGACCACGGT
CACCGTCTCCTCAGCTAGCACCAAGGGCCCATGCGTCTTCCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAGAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTATGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAAGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGAGGAGATGACCAAGAACCAAGT
CAGCCTGACCTGCCTGGTCAAAGGCTTCTATCCCAGCGACATCTGCGTGG
AGTGGGAGAGCAATGGGCAGCCGGAGAACAACTACAAGACCACGCCTCCC
GTGCTGGACTCCGACGGCTCCTTCTTCCTCTATTCCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGC
AAA
LC DNA for Ab5
SEQ ID NO: 29
GACATCCAGATGACCCAGTCTCCATCCTCCCTGTCTGCATCTGTAGGAGA
CAGAGTCACCATCACTTGCCAGGCGAGTCAGGGCATTCGCAACTATTTAA
ATTGGTATCAGCAGAAACCAGGGAAAGCCCCTAAGCTCCTGATCTACGAT
GCATCCAATTTGGAAACAGGGGTCCCATCAAGGTTCAGTGGAAGTGGATC
TGGGACAGATTTTACTTTCACCATCAGCAGCCTGCAGCCTGAAGATATTG
CAACATATTACTGTCAACAGTATGATAAGCTCCCGCTCACTTTCGGCGGA
GGGACCAAGGTGGAGATCAAACGGACCGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGC
Ab6
HCDR1 for Ab1, Ab2, and Ab6
SEQ ID NO: 1
GYTFTGYYIH
HCDR2 for Ab1, Ab2, and Ab6
SEQ ID NO: 2
WINPYTGGTNYAQKFQG
HCDR3 for Ab6
SEQ ID NO: 30
DIYGSSNYGGDV
LCDR1 for Ab6
SEQ ID NO: 31
QASQDISNYLN
LCDR2 for Ab1, Ab2, Ab3, Ab4, Ab5, and Ab6
SEQ ID NO: 5
DASNLET
LCDR3 for Ab6
SEQ ID NO: 32
QQYDTLPLT
VH for Ab6
SEQ ID NO: 33
QVQLVQSGAEVKKPGASVKVSCKASGYTFTGYYIHWVRQAPGQGLEWMGW
INPYTGGTNYAQKFQGRVTMTRDTSISTAYMELSRLRSDDTAVYYCARDI
YGSSNYGGDVWGQGTTVTVSS
VL for Ab6
SEQ ID NO: 34
DIQMTQSPSSLSASVGDRVTITCQASQDISNYLNWYQQKPGKAPKLLIYD
ASNLETGVPSRFSGSGSGTDFTFTISSLQPEDIATYYCQQYDTLPLTFGG
GTKVEIK
HC for Ab6
SEQ ID NO: 35
QVQLVQSGAEVKKPGASVKVSCKASGYTFTGYYIHWVRQAPGQGLEWMGW
INPYTGGTNYAQKFQGRVTMTRDTSISTAYMELSRLRSDDTAVYYCARDI
YGSSNYGGDVWGQGTTVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K
LC for Ab6
SEQ ID NO: 36
DIQMTQSPSSLSASVGDRVTITCQASQDISNYLNWYQQKPGKAPKLLIYD
ASNLETGVPSRFSGSGSGTDFTFTISSLQPEDIATYYCQQYDTLPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
HC DNA for Ab6
SEQ ID NO: 37
CAGGTGCAGCTGGTGCAGTCTGGGGCTGAGGTGAAGAAGCCTGGGGCCTC
AGTGAAGGTCTCCTGCAAGGCTTCTGGATACACCTTCACCGGCTACTATA
TACACTGGGTGCGACAGGCCCCTGGACAAGGGCTTGAGTGGATGGGATGG
ATCAACCCTTACACCGGTGGCACAAACTATGCACAGAAGTTTCAGGGCAG
GGTCACCATGACCAGGGACACGTCCATCAGCACAGCCTACATGGAGCTGA
GCAGGCTGAGATCTGACGACACGGCCGTGTATTACTGTGCGAGAGATATC
TATGGTTCGAGTAATTACGGTGGCGACGTCTGGGGCCAAGGGACCACGGT
CACCGTCTCCTCAGCTAGCACCAAGGGCCCATCGGTCTTCCCCCTGGCAC
CCTCCTCCAAGAGCACCTCTGGGGGCACAGCGGCCCTGGGCTGCCTGGTC
AAGGACTACTTCCCCGAACCGGTGACGGTGTCGTGGAACTCAGGCGCCCT
GACCAGCGGCGTGCACACCTTCCCGGCTGTCCTACAGTCCTCAGGACTCT
ACTCCCTCAGCAGCGTGGTGACCGTGCCCTCCAGCAGCTTGGGCACCCAG
ACCTACATCTGCAACGTGAATCACAAGCCCAGCAACACCAAGGTGGACAA
GAGAGTTGAGCCCAAATCTTGTGACAAAACTCACACATGCCCACCGTGCC
CAGCACCTGAACTCCTGGGGGGACCGTCAGTCTTCCTCTTCCCCCCAAAA
CCCAAGGACACCCTCATGATCTCCCGGACCCCTGAGGTCACATGCGTGGT
GGTGGACGTGAGCCACGAAGACCCTGAGGTCAAGTTCAACTGGTATGTGG
ACGGCGTGGAGGTGCATAATGCCAAGACAAAGCCGCGGGAGGAGCAGTAC
AACAGCACGTACCGTGTGGTCAGCGTCCTCACCGTCCTGCACCAAGACTG
GCTGAATGGCAAGGAGTACAAGTGCAAGGTCTCCAACAAAGCCCTCCCAG
CCCCCATCGAGAAAACCATCTCCAAAGCCAAAGGGCAGCCCCGAGAACCA
CAGGTGTACACCCTGCCCCCATCCCGGGAGGAGATGACCAAGAACCAAGT
CAGCCTGACCTGCCTGGTCAAAGGCTTCTATCCCAGCGACATCGCCGTGG
AGTGGGAGAGCAATGGGCAGCCGGAGAACAACTACAAGACCACGCCTCCC
GTGCTGGACTCCGACGGCTCCTTCTTCCTCTATTCCAAGCTCACCGTGGA
CAAGAGCAGGTGGCAGCAGGGGAACGTCTTCTCATGCTCCGTGATGCATG
AGGCTCTGCACAACCACTACACGCAGAAGAGCCTCTCCCTGTCTCCGGGC
AAA
LC DNA for Ab6
SEQ ID NO: 38
GACATCCAGATGACCCAGTCTCCATCCTCCCTGTCTGCATCTGTAGGAGA
CAGAGTCACCATCACTTGCCAGGCGAGTCAGGACATTAGCAACTATTTAA
ATTGGTATCAGCAGAAACCAGGGAAAGCCCCTAAGCTCCTGATCTACGAT
GCATCCAATTTGGAAACAGGGGTCCCATCAAGGTTCAGTGGAAGTGGATC
TGGGACAGATTTTACTTTCACCATCAGCAGCCTGCAGCCTGAAGATATTG
CAACATATTACTGTCAACAGTATGATACCCTCCCGCTCACTTTCGGCGGA
GGGACCAAGGTGGAGATCAAACGGACCGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGC
Human TNFα protein
SEQ ID NO: 39
MSTESMIRDVELAEEALPKKTGGPQGSRRCLFLSLFSFLIVAGATTLFCL
LHFGVIGPQREEFPRDLSLISPLAQAVRSSSRTPSDKPVAHVVANPQAEG
QLQWLNRRANALLANGVELRDNQLVVPSEGLYLIYSQVLFKGQGCPSTHV
LLTHTISRIAVSYQTKVNLLSAIKSPCQRETPEGAEAKPWYEPIYLGGVF
QLEKGDRLSAEINRPDYLDFAESGQVYFGIIAL
Rhesus macaque TNFα protein
SEQ ID NO: 40
MSTESMIRDVELAEEALPRKTAGPQGSRRCWFLSLFSFLLVAGATTLFCL
LHFGVIGPQREEFPKDPSLISPLAQAVRSSSRTPSDKPVAHVVANPQAEG
QLQWLNRRANALLANGVELTDNQLVVPSEGLYLIYSQVLFKGQGCPSNHV
LLTHTISRIAVSYQTKVNLLSAIKSPCQRETPEGAEAKPWYEPIYLGGVF
QLEKGDRLSAEINLPDYLDFAESGQVYFGIIAL
Canine TNFα protein
SEQ ID NO: 41
MSTESMIRDVELAEEPLPKKAGGPPGSRRCFCLSLFSFLLVAGATTLFCL
LHFGVIGPQREELPNGLQLISPLAQTVKSSSRTPSDKPVAHVVANPEAEG
QLQWLSRRANALLANGVELTDNQLIVPSDGLYLIYSQVLFKGQGCPSTHV
LLTHTISRFAVSYQTKVNLLSAIKSPCQRETPEGTEAKPWYEPIYLGGVF
QLEKGDRLSAEINLPNYLDFAESGQVYFGIIAL
Cynomolgus monkey TNFα protein
SEQ ID NO: 42
MSTESMIQDVELAEEALPRKTAGPQGSRRCWFLSLFSFLLVAGAATLFCL
LHFGVIGPQREEFPKDPSLISPLAQAVRSSSRTPSDKPVAHVVANPQAEG
QLQWLNRRANALVANGVELTDNQLVVPSEGLYLIYSQVLFKGQGCPSNHV
LLTHTISRIAVSYQTKVNLLSAIKSPCQRETPEGAEAKPWYEPIYLGGVF
QLEKGDRLSAEINLPDYLDFAESGQVYFGIIAL
LCDR1 consensus sequence
SEQ ID NO: 43
QASQGIXaa₇NYLN
Wherein Xaa₇ is Serine or Arginine
LCDR3 consensus sequence
SEQ ID NO: 44
QQYDXaa₅LPLT
Wherein Xaa5 is Asparagine or Lysine
human TNFR1
SEQ ID NO: 45
MGLSTVPDLLLPLVLLELLVGIYPSGVIGLVPHLGDREKRDSVCPQGKYI
HPQNNSICCTKCHKGTYLYNDCPGPGQDTDCRECESGSFTASENHLRHCL
SCSKCRKEMGQVEISSCTVDRDTVCGCRKNQYRHYWSENLFQCFNCSLCL
NGTVHLSCQEKQNTVCTCHAGFFLRENECVSCSNCKKSLECTKLCLPQIE
NVKGTEDSGTTVLLPLVIFFGLCLLSLLFIGLMYRYQRWKSKLYSIVCGK
STPEKEGELEGTTTKPLAPNPSFSPTPGFTPTLGFSPVPSSTFTSSSTYT
PGDCPNFAAPRREVAPPYQGADPILATALASDPIPNPLQKWEDSAHKPQS
LDTDDPATLYAVVENVPPLRWKEFVRRLGLSDHEIDRLELQNGRCLREAQ
YSMLATWRRRTPRREATLELLGRVLRDMDLLGCLEDIEEALCGPAALPPA
PSLLR
human TNFR2
SEQ ID NO: 46
MAPVAVWAALAVGLELWAAAHALPAQVAFTPYAPEPGSTCRLREYYDQTA
QMCCSKCSPGQHAKVFCTKTSDTVCDSCEDSTYTQLWNWVPECLSCGSRC
SSDQVETQACTREQNRICTCRPGWYCALSKQEGCRLCAPLRKCRPGFGVA
RPGTETSDVVCKPCAPGTFSNTTSSTDICRPHQICNVVAIPGNASMDAVC
TSTSPTRSMAPGAVHLPQPVSTRSQHTQPTPEPSTAPSTSFLLPMGPSPP
AEGSTGDFALPVGLIVGVTALGLLIIGVVNCVIMTQVKKKPLCLQREAKV
PHLPADKARGTQGPEQQHLLITAPSSSSSSLESSASALDRRAPTRNQPQA
PGVEASGAGEARASTGSSDSSPGGHGTQVNVTCIVNVCSSSDHSSQCSSQ
ASSTMGDTDSSPSESPKDEQVPFSKEECAFRSQLETPETLLGSTEEKPLP
LGVPDAGMKPS

What is claimed:

1. A conjugate of the Formula: 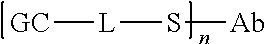
wherein Ab is an antibody that binds human TNFα, wherein Ab comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1, or 22;
the HCDR2 comprises SEQ ID NO: 2, or 23;
the HCDR3 comprises SEQ ID NO: 3, 13, or 30;
the LCDR1 comprises SEQ ID NO: 4, 14, 31, or 43;
the LCDR2 comprises SEQ ID NO: 5; and
the LCDR3 comprises SEQ ID NO: 6, 15, 32, or 44;
wherein 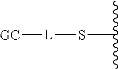
is: 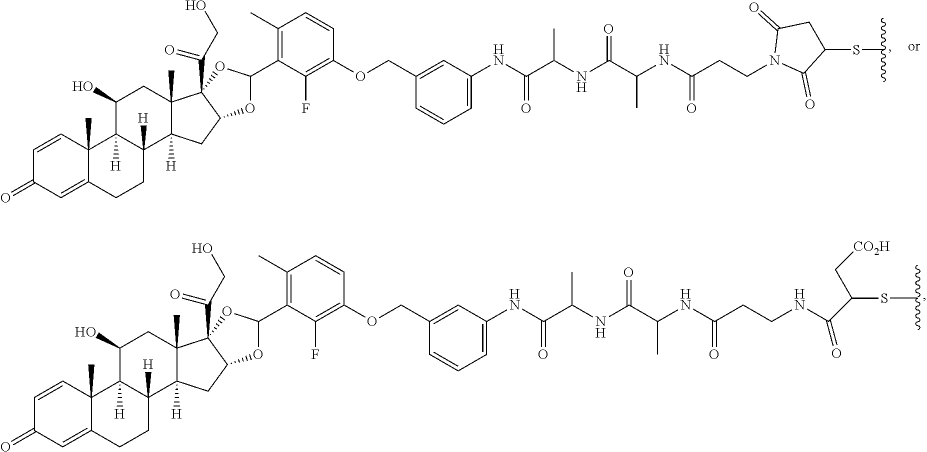
and n is 1-5.

2. The conjugate of claim 1, wherein 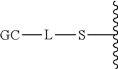
is:

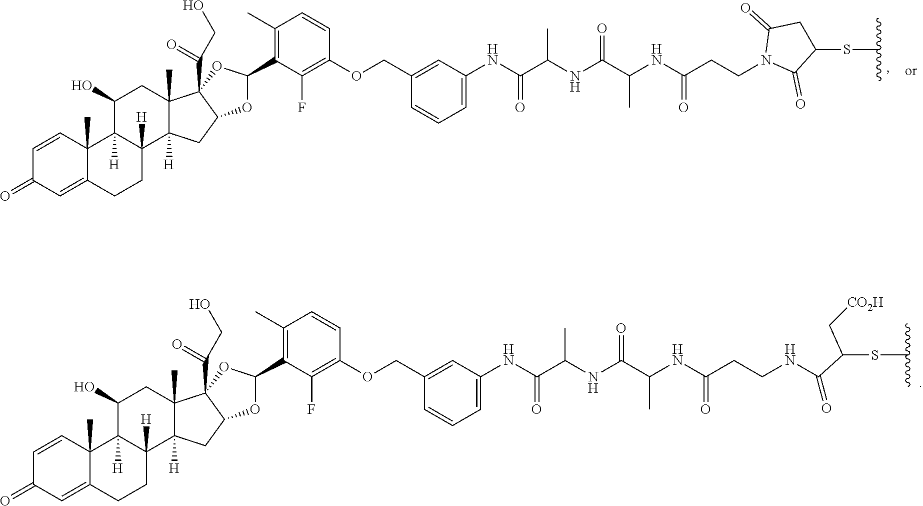

3. The conjugate of claim 1, wherein 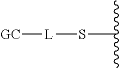
is:

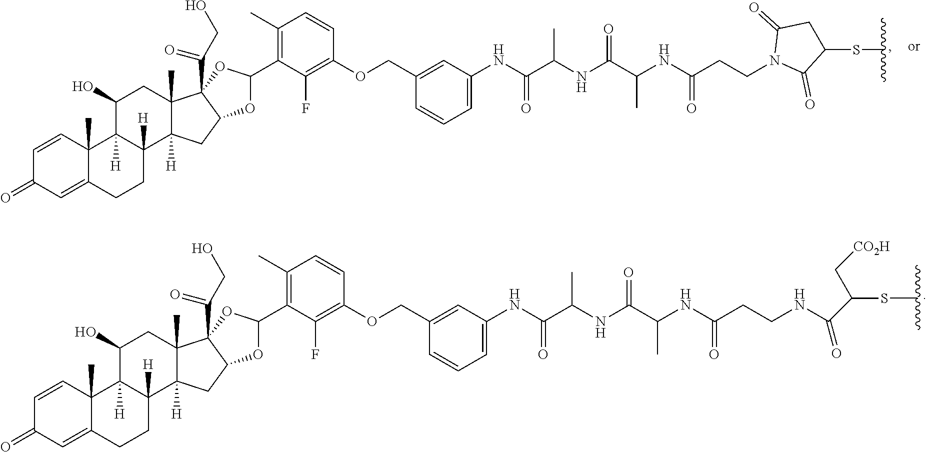

4. The conjugate of claim 1, wherein 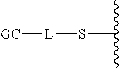
is : 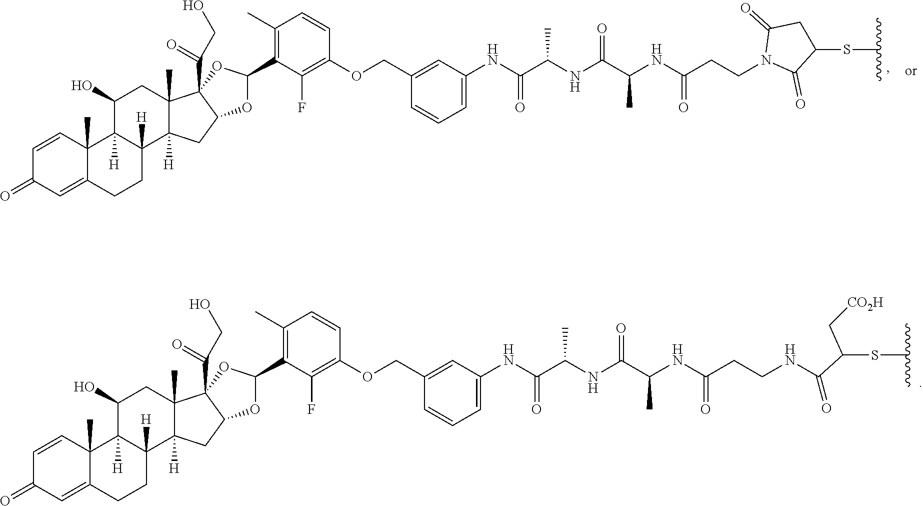
10 5. The conjugate of claim 1, wherein 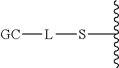
is:

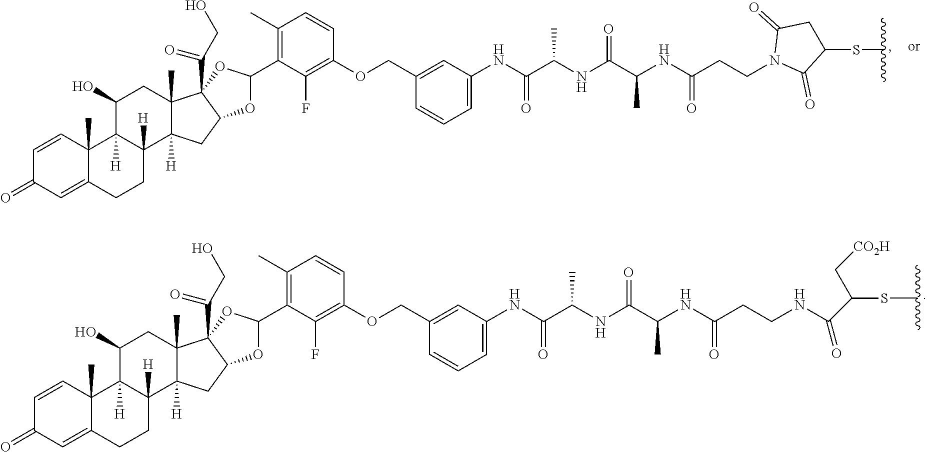

6. The conjugate of claim 1, wherein 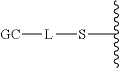
is:

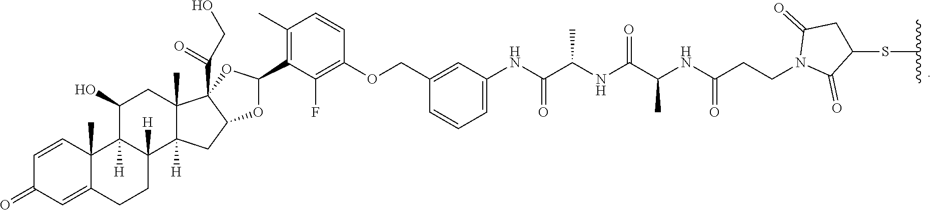

7. The conjugate of claim 1, wherein 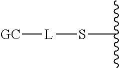
is:

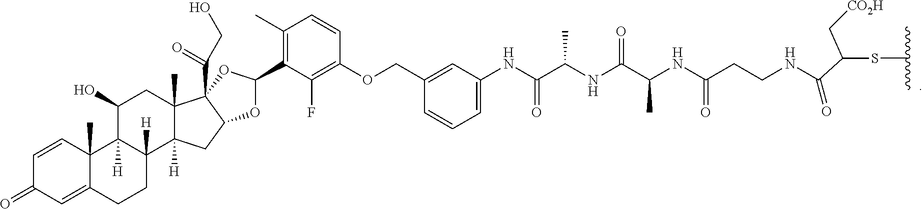

8. The conjugate of claim 1, wherein 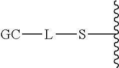
is:

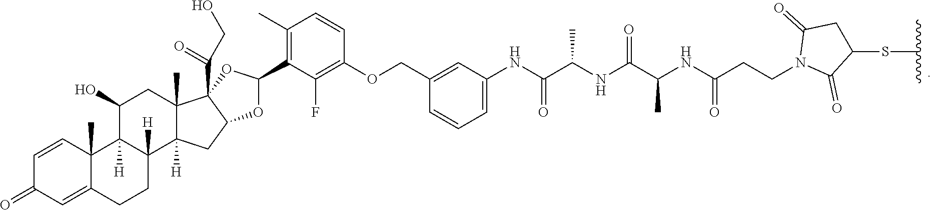

9. The conjugate of claim 1, wherein 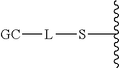
is:

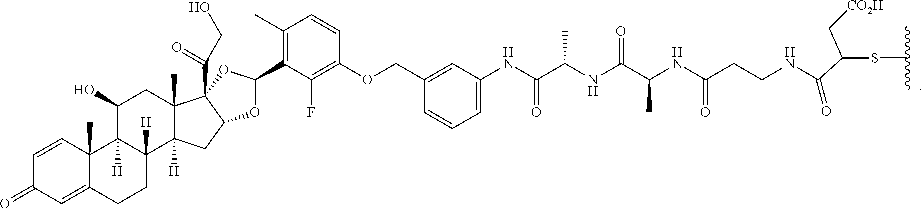

10. The conjugate of claim 1, wherein the antibody that binds human TNFα comprises heavy chain variable region (VH) and light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1;
the HCDR2 comprises SEQ ID NO: 2;
the HCDR3 comprises SEQ ID NO: 3;
the LCDR1 comprises SEQ ID NO: 4;
the LCDR2 comprises SEQ ID NO: 5; and
the LCDR3 comprises SEQ ID NO: 6.

11. The conjugate of claim 10, wherein the VH comprises SEQ ID NO: 7 and the VL comprises SEQ ID NO: 8.

12. The conjugate of claim 10, wherein the antibody comprises a heavy chain (HC) and a light chain (LC), wherein the HC comprises SEQ ID NO: 9 and the LC comprises SEQ ID NO: 10.

13. The conjugate of claim 1, wherein the antibody that binds human TNFα comprises heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1;
the HCDR2 comprises SEQ ID NO: 2;
the HCDR3 comprises SEQ ID NO: 13;
the LCDR1 comprises SEQ ID NO: 14;
the LCDR2 comprises SEQ ID NO: 5; and
the LCDR3 comprises SEQ ID NO: 15.

14. The conjugate of claim 13, wherein the VH comprises SEQ ID NO: 16 and the VL comprises SEQ ID NO: 17.

15. The conjugate of claim 13, wherein the antibody comprises a heavy chain (HC) and a light chain (LC), wherein the HC comprises SEQ ID NO: 18 and the LC comprises SEQ ID NO: 19.

16. The conjugate of claim 1, wherein the antibody that binds human TNFα comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 22;
the HCDR2 comprises SEQ ID NO: 23;
the HCDR3 comprises SEQ ID NO: 13;
the LCDR1 comprises SEQ ID NO: 4;
the LCDR2 comprises SEQ ID NO: 5; and
the LCDR3 comprises SEQ ID NO: 6.

17. The conjugate of claim 1, wherein the antibody that binds human TNFα comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 22;
the HCDR2 comprises SEQ ID NO: 23;
the HCDR3 comprises SEQ ID NO: 13;
the LCDR1 comprises SEQ ID NO: 14;
the LCDR2 comprises SEQ ID NO: 5; and
the LCDR3 comprises SEQ ID NO: 15.

18. The conjugate of claim 1, wherein the antibody that binds human TNFα comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 22;
the HCDR2 comprises SEQ ID NO: 23;
the HCDR3 comprises SEQ ID NO: 13;
the LCDR1 comprises SEQ ID NO: 14;
the LCDR2 comprises SEQ ID NO: 5; and
the LCDR3 comprises SEQ ID NO: 6.

19. The conjugate of claim 16, wherein the VH comprises SEQ ID NO: 24 and the VL comprises SEQ ID NO: 8.

20. The conjugate of claim 17, wherein the VH comprises SEQ ID NO: 24 and the VL comprises SEQ ID NO: 17.

21. The conjugate of claim 18, wherein the VH comprises SEQ ID NO: 24 and the VL comprises SEQ ID NO: 27.

22. The conjugate of claim 16, wherein the antibody comprises a heavy chain (HC) and a light chain (LC), wherein the HC comprises SEQ ID NO: 25 and the LC comprises SEQ ID NO: 10.

23. The conjugate of claim 17, wherein the antibody comprises a heavy chain (HC) and a light chain (LC), wherein the HC comprises SEQ ID NO: 25 and the LC comprises SEQ ID NO: 19.

24. The conjugate of claim 18, wherein the antibody comprises a heavy chain (HC) and a light chain (LC), wherein the HC comprises SEQ ID NO: 25 and the LC comprises SEQ ID NO: 28.

25. The conjugate of claim 1, wherein the antibody that binds human TNFα comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein:
the HCDR1 comprises SEQ ID NO: 1;
the HCDR2 comprises SEQ ID NO: 2;
the HCDR3 comprises SEQ ID NO: 30;
the LCDR1 comprises SEQ ID NO: 31;
the LCDR2 comprises SEQ ID NO: 5; and
the LCDR3 comprises SEQ ID NO: 32.

26. The conjugate of claim 25, wherein the VH comprises SEQ ID NO: 33 and the VL comprises SEQ ID NO: 34.

27. The conjugate of claim 25, wherein the antibody comprises a heavy chain (HC) and a light chain (LC), wherein the HC comprises SEQ ID NO: 35 and the LC comprises SEQ ID NO: 36.

28. The conjugate of claim 1, wherein the antibody comprises a heavy chain and a light chain, wherein the heavy chain comprises:
a cysteine at amino acid residue 124 (EU numbering);
a cysteine at amino acid residue 378 (EU numbering); or
a cysteine at amino acid residue 124 (EU numbering) and a cysteine at amino acid residue 378 (EU numbering).

29. The conjugate of claim 1, wherein n is 2-5.

30. The conjugate of claim 1, wherein n is 3-4.

31. A pharmaceutical composition comprising the conjugate of claim 1 and one or more pharmaceutically acceptable carrier, diluent, or excipient.

32. A method of treating an autoimmune disease or an inflammatory disease in a subject in need thereof, comprising administering to the subject an effective amount of the conjugate of claim 1.

33. A method of treating an autoimmune disease or an inflammatory disease in a subject in need thereof, comprising administering to the subject an effective amount of the pharmaceutical composition of claim 31.

34. The method of claim 32, wherein the autoimmune disease or the inflammatory disease is Rheumatoid Arthritis (RA), Juvenile Idiopathic Arthritis, Psoriatic Arthritis (PsA), Ankylosing Spondylitis (AS), Crohn's Disease (CD), Ulcerative Colitis (UC), Plaque Psoriasis (PS), Hidradenitis Suppurativa (HS), Uveitis, Non-Infectious Intermediate, Posterior, Pan Uveitis, Behcet's Disease, or Polymyalgia Rheumatica (PMR).

35. The method of claim 32, wherein the subject being administered the effective amount of the conjugate received a prior treatment of other anti-TNFα therapeutic, and wherein the subject developed anti-drug antibodies against the other anti-TNFα therapeutic, wherein the other anti-TNFα therapeutic is adalimumab, infliximab, golimumab, certolizumab, and Etanercept, or a conjugate thereof.

36. A compound, wherein the compound is:

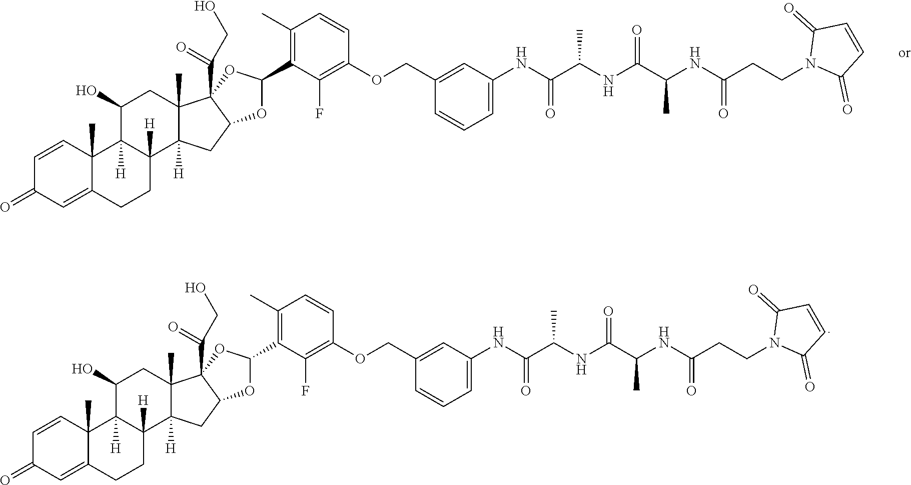

37. A compound of the formula:

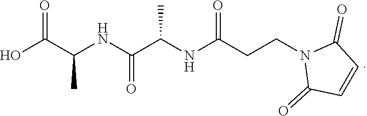 [SMILES: C[C@H](NC(=O)[C@H](C)NC(=O)CCN1C(=O)C=CC1=O)C(=O)O]

38. A method of producing a conjugate, the method comprising contacting the compound of claim 36 with an anti-human TNFα antibody.

39. The method of claim 38, wherein the conjugate is produced following the steps comprising:
(a) reducing an anti-human TNFα antibody with a reducing agent, wherein the anti-human TNFα antibody comprises one or more engineered cysteine residue;
(b) oxidizing the anti-human TNFα antibody with an oxidizing reagent; and
(c) contacting the compound of claim 37 with the anti-human TNFα antibody to produce the conjugate.

40. The method of claim 39, wherein the reducing agent is dithiothreitol and the oxidizing agent is dehydroascorbic acid.
